(19) United States
(12) Patent Application Publication
(10) Pub. No.: US 2007/0254894 A1
(43) Pub. Date: Nov. 1, 2007

(54) Novel small molecules with selective cytotoxicity against human microvascular endothelial cell proliferation

(76) Inventors: John L. Kane JR., Maynard, MA (US); Bradford H. Hirth, Littleton, MA (US); Christopher Yee, Needham, MA (US); Mark M. Staveski, Taunton, MA (US); Hans-Peter Biemann, Cambridge, MA (US); Marko J. Pregel, Arlington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/651,406

(22) Filed: Jan. 9, 2007

Related U.S. Application Data
(60) Provisional application No. 60/758,108, filed on Jan. 10, 2006.

(51) Int. Cl. A61K 31/4709; A61K 31/497; A61P 9/00; C07D 215/38; C07D 243/08; C07D 401/14; C07D 413/14

(52) U.S. Cl. 514/253.06; 514/314; 540/575; 544/128; 544/238; 544/353; 544/363; 546/171

(57) ABSTRACT

Disclosed herein are angiogenesis inhibitors represented by formula (I) or formula (II): 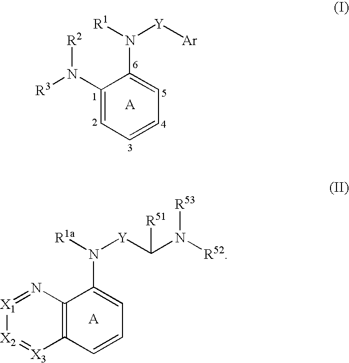 The variables for formulas (I) and (II) are defined herein.

### RELATED APPLICATION(S)

This application claims the benefit of U.S. Provisional Application No. 60/758,108, filed on Jan. 10, 2006.

The entire teachings of the above application(s) are incorporated herein by reference.

### BACKGROUND OF THE INVENTION

Angiogenesis, or the formation of new blood vessels, is a strictly regulated process in healthy tissue. Aside from normal wound healing and reproductive events, the rate of neovascularization is minimal in adults. Hyperproliferation of blood vessels, however, is associated with several disease states (e.g., diabetic retinopathy, wet macular degeneration, psoriasis, rheumatoid arthritis and angiomas) or facilitate the metastasis and growth of tumors. The induction of angiogenesis involves several sequential steps. One of the key events in this process is the proliferation of endothelial cells, which ultimately differentiate into mature blood vessels.

Agents which inhibit the proliferation of endothelial cells therefore have the potential to treat conditions characterized by the hyperproliferation of blood vessels. Unfortunately, most agents which affect endothelial cell proliferation are non-selective, e.g., are cytotoxic to cells generally. Thus, there is a need for agents which act selectively against endothelial cells.

### SUMMARY OF THE INVENTION

It has now been found that 2-pyridine-carboxylic acid quinolin-8-ylamide compounds are selective cytotoxic agents against human microvascular endothelial cells (HMVECs). Specifically, HMVECs stimulated to proliferate with basic fibroblast growth factor or vascular endothelial growth factor are vulnerable to the toxicity of these compounds, while quiescent HMVECs and other cell types (e.g., dermal, fibroblasts, kidney epithelial, MCF-7, SF268 and H460) are not (see biological data provided below). In addition, several of these compounds were tested and found to significantly inhibit neovascularization in a mouse model of oxygen induced retinopathy and to inhibit the lateral migration of HMVECs over a plastic cell culture substratum. Based on this discovery, novel angiogenesis inhibitors, pharmaceutical compositions comprising the angiogenesis inhibitors and methods of treatment using the angiogenesis inhibitors are disclosed herein.

One embodiment of the present invention is a compound of Structure Formula (I): 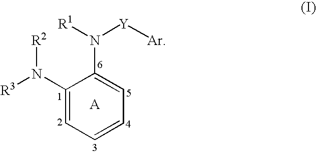
Pharmaceutically acceptable salts of the compound of Structural Formula (I) are also included.

Ring A is optionally substituted at any one or more substitutable ring carbon atoms.

—Y— is —SO₂— or —C(O)—.

Ar is an optionally substituted monocyclic aryl group.

R¹ is H or a C1-C6 alkyl group optionally substituted with amino, hydroxyl, methoxy or ethoxy.

R² and R³ are independently C1-C6 alkyl optionally substituted with amino, hydroxyl, methoxy or ethoxy; or

R₂ is absent and R₃, taken together with the nitrogen atom to which it is bonded and C1 and C2 of Ring A, forms an optionally substituted six membered nitrogen-containing aromatic group referred to as “Ring B”.

Each substituent on Ring A, B and Ar is independently selected.

In certain instances, at least one of Ring A, Ring B and Ar is substituted with a group other than alkyl when Ar is pyridyl; and Ar is not substituted with a carboxamide when Ar is phenyl or pyridyl. In these instances, the term “carboxamide” is understood to mean —C(O)NR₂ wherein R is an optionally substituted alkyl or aryl group.

Another embodiment of the invention is an angiogenesis inhibitor represented by Structural Formula (II):

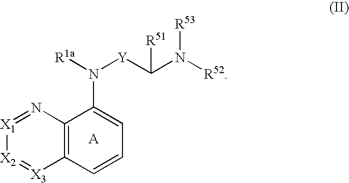

Pharmaceutically acceptable salts of the compound of Structural Formula (II) are also included.

Ring A is optionally substituted at any substitutable ring carbon atom.

Y is —C(O)— or —S(O)₂—;

X1-X₃ are independently N, CH or CR^{6a}, provided that X₁ and X₂ are not both N.

R^{1a} is H or a C1-C6 alkyl group optionally substituted with amino, hydroxyl, methoxy or ethoxy.

Each R^{6a} is independently halogen, alkyl, haloalkyl, —OR²⁰, —O(haloalkyl), —SR²⁰, —NO₂, —CN, —N(R²¹)₂, —NR²¹C(O)R²⁰, —NR²¹CO₂R²², —N(R²¹)C(O)N(R²¹)₂, —C(O)R²⁰, —C(O)N(R²¹)₂, —S(O)₂R²⁰, —SO₂N(R²¹)₂, —S(O)R²², —NR²¹SO₂N(R²¹)₂, —NR²¹SO₂R²², —Vₒ—N(R²¹)₂, —O—Vₒₒ—N(R²¹)₂, —S—Vₒₒ—N(R²¹)₂ or —N(R²¹)—Vₒₒ—N(R²¹)₂.

Each Vₒ is independently a C1-C4 alkylene group.

Each Vₒₒ is independently a C2-C4 alkylene group.

Each R²⁰ is independently hydrogen or an alkyl group.

Each R²¹ is independently hydrogen, an alkyl group, —CO₂R²⁰, —SO₂R²⁰ or —C(O)R²⁰; or —N(R²¹)₂ taken together is an optionally substituted non-aromatic nitrogen-containing heterocyclic group.

Each R²² is independently an alkyl group.

R⁵¹ and R⁵² taken together with their intervening atoms form an optionally substituted monocyclic non-aromatic heterocyclic group and R⁵³ is —H or C1-C6 alkyl group optionally substituted with amine, C1-C2 alkylamine, C1-C2 dialkylamine, hydroxyl, methoxy, ethoxy, cycloalkyl, or optionally substituted aryl; or R⁵¹ is C1-C6 alkyl group optionally substituted with amino, hydroxyl, methoxy, ethoxy or an optionally substituted aryl group (in some instances, —H is an additional value for R⁵¹); and R⁵² and R⁵³ are independently —H or C1 -C6 alkyl group optionally substituted with amino, hydroxyl, methoxy, ethoxy, cycloalkyl, or an optionally substituted aryl group; or —NR⁵²R⁵³ is an optionally substituted monocyclic non-aromatic heterocyclic group.

Another embodiment of the present invention is a method of inhibiting angiogenesis in a subject in need of such treatment. The method comprises the step of administering to the subject an effective amount of an angiogenesis inhibitor represented by Structural Formula (I) or (II) or a pharmaceutically acceptable salt thereof, provided that when the angiogenesis inhibitor is represented by Structural Formula (II), then —CO₂R²⁰ is an additional value for R^{6a}.

Another embodiment of the present invention is a pharmaceutical composition comprising a pharmaceutically acceptable carrier or diluent and an angiogenesis inhibitor represented by Structural Formula (I) or (II), or a pharmaceutically acceptable salt thereof. Preferably, the pharmaceutical composition is used in therapy.

Another embodiment of the present invention is the use of an angiogenesis inhibitor represented by Structural Formula (I) or (II) or a pharmaceutically acceptable salt thereof for the manufacture of a medicament for inhibiting angiogenesis in a subject in need of such treatment, provided that when the compound angiogenesis inhibitor is represented by Structural Formula (II), then —CO₂R²⁰ is an additional value for R^{6a}.

The disclosed compounds are selective cytotoxic agents against human microvascular endothelial cells while having little or no harmful effects against other cell types. As such, they can be used as angiogenesis inhibitors with minimal side effects. As such, they have potential to be effective medications against conditions such as cancer, diabetic retinopathy, wet macular degeneration, psoriasis, rheumatoid arthritis and angiomas.

### BRIEF DESCRIPTION OF THE FIGURES

FIG. 1 is a bar graph showing the percent reduction in ocular neovascularization in a mouse model resulting from intravitreal administration of Compound 9, Compound 98 and DMSO. The reduction is relative to a vehicle treated eye.

FIG. 2 is a bar graph showing the number of animals having a greater than 50% reduction in the number of neovascular events in a mouse model resulting from intravitreal administration of Compound 9, Compound 98 and DMSO. The reduction is determined relative to un-injected littermate controls.

### DETAILED DESCRIPTION OF THE INVENTION

The present invention is directed to angiogenesis inhibitors represented by Structural Formulas (I) and (II), or a pharmaceutically acceptable salt thereof, and the use of such compounds for inhibiting angiogenesis in a subject in need of such treatment. Definitions of the terms used to describe these inventions are provided below.

The disclosed angiogenesis inhibitors can be used to treat wet age-related macular-degeneration (hereinafter “ARMD”). ARMD is a degenerative condition of the macula (the central retina) which can cause vision loss in those 50 or older. Its prevalence increases with age. ARMD is caused by hardening of the arteries that nourish the retina. This deprives the sensitive retinal tissue of oxygen and nutrients the retina needs to function and thrive. As a result, the central vision deteriorates. About 10% of patients who suffer from macular degeneration have wet AMD. This type occurs when new vessels form to improve the blood supply to oxygen-deprived retinal tissue. However, the new vessels are very delicate and break easily, causing bleeding and damage to surrounding tissue. The disclosed compounds can be used to inhibit this damaging formation of new blood vessels.

Diabetic retinopathy can also be treated with the disclosed angiogenesis inhibitors. Diabetic retinopathy is a complication of diabetes and a leading cause of blindness. It occurs when diabetes damages the tiny blood vessels inside the retina. At this earliest stage of diabetic retinopathy, microaneurysms occur. These are small areas of balloon-like swelling in the retina's tiny blood vessels. As the disease progresses, some blood vessels that nourish the retina are blocked, depriving several areas of the retina with their blood supply. These areas of the retina send signals to the body to grow new blood vessels for nourishment. At this advanced stage, the signals sent by the retina for nourishment trigger the growth of new blood vessels. This condition is called proliferative retinopathy. These new blood vessels are abnormal and fragile. They grow along the retina and along the surface of the clear, vitreous gel that fills the inside of the eye.

By themselves, these blood vessels do not cause symptoms or vision loss. However, they have thin, fragile walls. If they leak blood, severe vision loss and even blindness can result. Fluid can leak into the center of the macula, the part of the eye where sharp, straight-ahead vision occurs. The fluid makes the macula swell, blurring vision. This condition is called macular edema. The disclosed angiogenesis inhibitors can be used to inhibit the formation of these abnormal and fragile blood vessels.

Psoriasis can also be treated with the disclosed angiogenesis inhibitors. Psoriasis is a chronic skin disease occurring in approximately 3% of the population worldwide. It is characterized by excessive growth of the epidermal keratinocytes, inflammatory cell accumulation and excessive dermal angiogenesis. Alterations in the blood vessel formation of the skin are a prominent feature of psoriasis. Thus, angiogenesis inhibitors can be used to treat subject with this disease.

It is believed that the disclosed angiogenesis inhibitors can be used to treat (therapeutically or prophylactically) restenosis. This is based on a number of studies support the utility of inhibiting VEGF signaling to reduce restenosis: a) Shojima and Walsh, “The Role of Vascular Endothelial Growth Factor in Restenosis”, Circulation 110: 2283-2286 (2004); (b) Moulton et al., “Angiogenesis inhibitors endostatin or TNP-470 reduce intimal neovascularization and plaque growth in apolipoprotein E-deficient mice”, Circulation 99: 1726-1732 (1999); (c) Khurana et al., “Angiogenesis-dependent and independent phases of intimal hyperplasia”, Circulation 110: 2436-2443 (2004); (d) Ohtani et al., “Blockade of vascular endothelial growth factor suppresses experimental restenosis after intraluminal injury by inhibiting recruitment of monocyte lineage cells”, Circulation 110: 2444-2452 (2004).

Rheumatoid arthritis can also be treated with the disclosed angiogenesis inhibitors. The expansion of the synovial lining of joints in rheumatoid arthritis (RA) and the subsequent invasion by the pannus of underlying cartilage and bone necessitate an increase in the vascular supply to the synovium, to cope with the increased requirement for oxygen and nutrients. The formation of new blood vessels is a key event in the formation and maintenance of the pannus in RA. This pannus is highly vascularized. Disruption of the formation of new blood vessels would not only prevent delivery of nutrients to the inflammatory site, but could also lead to vessel regression and possibly reversal of disease. The disclosed angiogenesis inhibitors can be used to disrupt the formation of new blood vessels in subjects with this disease.

Angiogenesis performs a critical role in the development of cancer. Solid tumors smaller than 1 to 2 cubic millimeters are not vascularized. Once they reach the critical volume of 2 cubic millimeters, oxygen and nutrients have difficulty diffusing to the cells in the center of the tumor, causing a state of cellular hypoxia that marks the onset of tumoral angiogenesis. New blood vessel development is an important process in tumor progression. It favors the transition from hyperplasia to neoplasia i.e. the passage from a state of cellular multiplication to a state of uncontrolled proliferation characteristic of tumor cells. Neovascularization also influences the dissemination of cancer cells throughout the entire body eventually leading to metastasis formation. The vascularization level of a solid tumor is thought to be an excellent indicator of its metastatic potential.

Angiogenesis inhibitors deprive malignant tissue of its oxygen and nutrient supply; in addition, it is unable to eliminate metabolic wastes. This in turn inhibits tumor progression and metastatic progression that accompanies most advanced cancers. The disclosed angiogenesis inhibitors can be used for these purposes as a treatment for cancers. Examples of cancers which can be treated with the disclosed angiogenesis inhibitors include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma.

Because increased bone marrow (BM) angiogenesis has been demonstrated in hematologic malignancies, it is believed that the disclosed angiogenesis inhibitors will be particularly effective in treating multiple myeloma, chronic myeloid leukemia, acute myeloid or lymphocytic leukemia, chronic lymphocytic leukemia (CLL) as well as myelodysplastic syndromes. See Vacca et al. “Bone Marrow Angiogenesis and Progression in Multiple Myeloma Br J Haematol, 87:503-8 (1994); Aguayo et al., “Angiogenesis in Acute and Chronic Leukaemia and Myelodysplastic Syndromes. Blood 96:2240-5 (2000); Hussong et al., “Evidence of Increased Angiogenesis in Patients with Acute Myeloid Leukaemia, Blood 95:309-13 (2000); Perez-Atayde et al., “Spectrum of Tumor Angiogenesis in the Bone Marrow of Children with Acute Lymphoblastic Leukemia, Am J Pathol 150:815-21 (1997); Molica et al., “Prognostic Value of Enhanced Bone Marrow Angiogenesis in Early B-Cell Chronic Lymphocytic Leukemia, Blood 100:3344-51 (2002); and Cheson et al., “National Institute-Sponsored Working Group Guidelines for Chronic Lymphocytic Leukemia: Revised Guidelines for Diagnosis and Treatment, Blood 87”4990-7 (1996).

When used to treat or prevent cancer, the disclosed angiogenesis inhibitors can advantageously be combined with other anticancer agents. Examples include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-nl; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer drugs which can be combined with the disclosed angiogenesis inhibitors for the treatment or prevention of cancer include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-i; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine.; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzarmides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Examples-of therapeutic antibodies which can be combined with the disclosed angiogenesis inhibitors for the treatment or prevention of cancer include but are not limited to HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-1A cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Chemotherapeutic agents that can be combined with the disclosed angiogenesis inhibitors for the treatment or prevention of cancer include but are not limited to alkylating agents, antimetabolites, natural products, or hormones. Examples of alkylating agents useful for the treatment or prevention of T-cell malignancies in the methods and compositions of the invention include but are not limited to, nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites useful for the treatment or prevention of T-cell malignancies in the methods and compositions of the invention include but are not limited to folic acid analog (e.g., methotrexate), or pyrimidine analogs (e.g., Cytarabine), purine analogs (e.g., mercaptopurine, thioguanine, pentostatin). Examples of natural products useful for the treatment or prevention of T-cell malignancies in the methods and compositions of the invention include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents which can be combined with the disclosed angiogenesis inhibitors for the treatment or prevention of cancer include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.).

Examples of antimetabolites which can be combined with the disclosed angiogenesis inhibitors for the treatment or prevention of cancer include but are not limited to folic acid analog (e.g., methotrexate), or pyrimidine analogs (e.g., fluorouracil, floxouridine, Cytarabine), purine analogs (e.g., mercaptopurine, thioguanine, pentostatin).

Examples of natural products useful for the treatment or prevention of cancer which can be combined with the disclosed angiogenesis inhibitors include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha).

Examples of hormones and antagonists useful for-the treatment or prevention of cancer which can be combined with the disclosed angiogenesis inhibitors include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide).

Other agents which can be combined with the disclosed angiogenesis inhibitors for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimnide).

Other anticancer agents which can be used in combination with the disclosed angiogenesis inhibitors for the treatment or prevention of cancer are those which act by arresting cells in the G2-M phases due to stabilized microtubules. Examples of anti-cancer agents which act by arresting cells in the G2-M phases due to stabilized microtubules include without limitation the following marketed drugs and drugs in development: taxol, and analogs of taxol (e.g., taxotere) Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known-as LS-4577), LS-4578 (Pharmacia, also known as LS-477-P), LS-4477 (Pharmacia), LS-4559 (Pharmacia), RPR-1 12378 (Aventis), Vincristine sulfate, DZ-3358 (Daiichi), FR-1 82877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Arrmad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

The term “alkyl” as used herein means saturated straight-chain or branched hydrocarbon. An alkyl group is typically C₁₋₈, more typically C₁₋₆. The terms “alkyl”, “alkoxy”, “hydroxyalkyl”, “haloalkyl”, “aralkyl” “alkoxyalkyl”, “alkylamine”, “cycloalkylalkyl”, “dialkyamine”, “alkylamino”, “dialkyamino” “alkylcarbonyl”, “alkoxycarbonyl” and the like, used alone or as part of a larger moiety includes both straight and branched saturated chains containing one to eight carbon atoms.

The term “alkoxy” means —O-alkyl; “hydroxyalkyl” means alkyl substituted with hydroxy; “aralkyl” means alkyl substituted with an aryl group; “alkoxyalkyl” mean alkyl substituted with an alkoxy group; “alkylamine” means amine substituted with an alkyl group; “cycloalkylalkyl” means alkyl substituted with cycloalkyl; “dialkylamine” means amine substituted with two alkyl groups; “alkylcarbonyl” means —C(O)—R, wherein R is alkyl; “alkoxycarbonyl” means —C(O)—OR, wherein R is alkyl; and where alkyl is as defined above.

The terms “amine” and “amino” are used interchangeably throughout herein and mean —NH₂, —NHR or —NR₂, wherein R is alkyl.

“Cycloalkyl” means a saturated carbocyclic ring, with from three to eight carbons.

The terms “haloalkyl” and “haloalkoxy” means alkyl or alkoxy, as the case may be, substituted with one or more halogen atoms. The term “halogen” means F, Cl, Br or I. Preferably the halogen in a haloalkyl or haloalkoxy is F.

The term “acyl group” mean —C(O)R, wherein R is an optionally substituted alkyl group or aryl group (e.g., optionally substituted phenyl). R is preferably an unsubstituted alkyl group or phenyl.

An “alkylene group” is represented by —[CH₂]_{z}—, wherein z is a positive integer, preferably from one to eight, more preferably from one to four.

An atom or atoms is “intervening” between two groups when one must pass through the atom or atoms in moving from one of the groups to the second group along the scaffold of the molecule. For example, the nitrogen atom to which R⁵² is attached and the carbon atom to which R⁵¹ is attached are intervening with respect to R⁵¹ and R⁵².

The term “aromatic group” used alone or as part of a larger moiety as in “aralkyl”, “aralkoxy”, or “aryloxyalkyl”, includes carbocyclic aromatic rings and heteroaryl rings. The term “aromatic group” may be used interchangeably with the terms “aryl”, “aryl ring” “aromatic ring”, “aryl group” and “aromatic group”.

Carbocyclic aromatic rings have only carbon ring atoms (typically six to fourteen) and include monocyclic aromatic rings such as phenyl and fused polycyclic aromatic ring systems in which two or more carbocyclic aromatic rings are fused to one another. Examples include 1-naphthyl, 2-naphthyl, 1-anthracyl.

The term “heteroaryl”, “heteroaromatic”, “heteroaryl ring”, “heteroaryl group” and “heteroaromatic group”, used alone or as part of a larger moiety as in “heteroaralkyl” or “heteroarylalkoxy”, refers to aromatic ring groups having five to fourteen ring atoms selected from carbon and at least one heteroatom (e.g., oxygen, nitrogen or sulfur). They include monocyclic rings and polycyclic rings in which a monocyclic heteroaromatic ring is fused to one or more other carbocyclic aromatic or heteroaromatic rings. Examples of monocyclic heteroaryl groups include furanyl (e.g., 2-furanyl, 3-furanyl), imidazolyl (e.g., N-imidazolyl, 2-imidazolyl, 4-imidazolyl, 5-imidazolyl), isoxazolyl( e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), triazolyl (e.g., 2-triazolyl, 5-triazolyl), tetrazolyl (e.g., tetrazolyl) and thienyl (e.g., 2-thienyl, 3-thienyl. Examples of monocyclic six-membered nitrogen-containing heteraryl groups include pyrimidinyl, pyridinyl and pyridazinyl. Examples of polycyclic aromatic heteroaryl groups include carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, or benzisoxazolyl.

The term “non-aromatic heterocyclic group”, used alone or as part of a larger moiety as in “non-aromatic heterocyclylalkyl group”, refers to non-aromatic ring systems typically having five to twelve members, preferably five to seven, in which one or more ring carbons, preferably one or two, are each replaced by a heteroatom such as N, O, or S. A non-aromatic heterocyclic group can be monocyclic or fused bicyclic A “nitrogen-containing non-aromatic heterocyclic group” is a non-aromatic heterocyclic group with at least one nitrogen ring atom.

Examples of non-aromatic heterocyclic groups include (tetrahydrofuranyl (e.g., 2-tetrahydropyranyl, 3-tetrahydropyranyl, 4-tetrahydropyranyl), [1,3]-dioxalanyl, [1,3]-dithiolanyl, [1,3]-dioxanyl, tetrahydrothienyl (e.g., 2-tetrahydrothienyl, 3-tetrahydrothieneyl), azetidinyl (e.g., N-azetidinyl, 1-azetidinyl, 2-azetidinyl), oxazolidinyl (e.g., N-oxazolidinyl, 2-oxazolidinyl, 4-oxazolidinyl, 5-oxazolidinyl), morpholinyl (e.g., N-morpholinyl, 2-morpholinyl, 3-morpholinyl), thiomorpholinyl (e.g., N-thiomorpholinyl, 2-thiomorpholinyl, 3-thiomorpholinyl), pyrrolidinyl (e.g., N-pyrrolidinyl, 2-pyrrolidinyl, 3-pyrrolidinyl) piperazinyl (e.g., N-piperazinyl, 2-piperazinyl), piperidinyl (e.g., N-piperidinyl), 2-piperidinyl, 3-piperidinyl, 4-piperidinyl), thiazolidinyl (e.g., 4-thiazolidinyl), diazolonyl and N-substituted diazolonyl. The designation “N” on N-morpholinyl, N-thiomorpholinyl, N-pyrrolidinyl, N-piperazinyl, N-piperidinyl and the like indicates that the non-aromatic heterocyclic group is attached to the remainder of the molecule at the ring nitrogen atom.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two aromatic rings are fused. In addition, “substitutable ring atom” does not include ring carbon or nitrogen atoms when the structure depicts that they are already attached to a moiety other than hydrogen. Thus, the carbon atom bonded to —NR¹R² in Structural Formula (I) is not a “substitutable ring atom” within the meaning of the term, as it is used herein.

An aryl group may contain one or more substitutable ring atoms, each bonded to a suitable substituent. Examples of suitable substituents on a substitutable ring carbon atom of an aryl group include halogen, alkyl, haloalkyl, Ar^{A}, —OR^{A}, —O(haloalkyl), —SR^{A}, —NO₂, —CN, —N(R^{B})₂, —NR^{B}C(O)R^{A}, —NR^{B}CO₂R^{C}, —N(R^{B})C(O)N(R^{B})₂, —C(O)R^{A}, —CO₂R^{A}, —S(O)₂R^{A}, —SO₂N(R^{B})₂, —S(O)R^{C}, —NR^{B}SO₂N(R^{B})₂, —NR^{B}SO₂R^{C}, —V_{A}—Ar^{A}, —V_{A}—OR^{A}, —V—O(haloalkyl), —V_{A}—SR^{A}, —V_{A}—NO₂, —V_{A}—CN, —V_{A}—N(R^{B})₂, —V_{A}—NR^{B}C(O)R^{A}, —V_{A}—NR^{B}CO₂R^{C}, —V_{A}—N(R^{B})C(O)N(R^{B})₂, —V_{A}—C(O)R^{A}, —V_{A}—CO₂R^{A}, —V_{A}—S(O)₂R^{A}, —V_{A}—SO₂N(R^{B})₂, —V_{A}—S(O)R^{C}, —V_{A}—NR^{B}SO₂N(R^{B})₂, —V_{A}—NR^{B}SO₂R^{C}, —O—V_{A}—Ar^{A}, —O—V_{B}—N(R^{B})₂, —S—V_{A}—Ar^{A}, —S—V_{B}—N(R^{B})₂, —N(R^{B})—V_{B}—Ar^{A}, —N(R^{B})—V_{B}—N(R^{B})₂, —NR^{B}C(O)—V_{A}—N(R^{B})₂, —NR^{B}C(O)—V_{A}—Ar^{A}, —C(O)—V_{A}—N(R^{B})₂, —C(O)—V_{A}—Ar^{A}, —CO₂—V_{B}—N(R^{B})₂, —CO₂—V_{A}—Ar^{A}, —C(O)N(R^{B})—V_{B}—N(R^{B})₂, —C(O)N(R^{B})—V_{A}—Ar^{A}, —S(O)₂—V_{A}—N(R^{B})₂, —S(O)₂—V_{A—Ar}^{A}, —SO₂N(R^{B})—V_{B}—N(R^{B})₂, —SO₂N(R^{b})—V_{A}—Ar^{A}, —S(O)—V_{A}—N(R^{B})₂, —S(O)—V_{A}—Ar^{A}, —NR^{B}SO₂—V_{A}—N(R^{B})₂ or —NR^{B}SO₂—V_{A}—Ar^{A}; or two adjacent substituents, taken together, form a methylenedioxy, ethylenedioxy or —[CH₂]₄— group.

Each V_{A} is independently a C1-C4 alkylene group.

Each V_{B} is independently a C2-C4 alkylene group.

Ar^{A} is a monocyclic aromatic group each substituted with zero, one or two groups independently selected from halogen, alkyl, amino, alkylamino, dialkylamino, alkoxy, nitro, cyano, hydroxy, haloalkoxy or haloalkyl.

Each R^{A} is independently i) hydrogen; ii) an aromatic group substituted with zero, one or two groups represented by halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R^{B} is independently R_{A}, —CO₂R^{A}, —SO₂R^{A} or —C(O)R^{A}; or —N(R^{B})₂ taken together is an optionally substituted non-aromatic heterocyclic group.

Each R^{C} is independently: i) an aromatic group substituted with zero, one or two groups represented by halogen, alkyl, amino, alkylamino, dialkylamino, alkoxy, nitro, cyano, hydroxy, haloalkoxy or haloalkyl; or ii) an alkyl group optionally substituted with halogen, hydroxyl, alkoxy, nitro, cyano, alkoxycarbonyl, alkylcarbonyl or haloalkoxy.

An alkyl or a non-aromatic heterocyclic group (including, but not limited to, non-aromatic heterocyclic groups represented by —N(R²¹)₂, —N(R³¹)₂, —N(R⁴¹)₂, —NR⁵¹N⁵², the group formed from R⁵¹ and R⁵² and their intervening atoms and —N(R^{B})₂) may contain one or more substituents. Examples of suitable substituents for an alkyl or a ring carbon of a non-aromatic heterocyclic group include those listed above for a substitutable carbon of an aryl and the following: ═O, ═S, ═NNHR^{C}, ═NN(R^{C})₂, ═NNHC(O)R^{C}, ═NNHCO₂ (alkyl), ═NNHSO₂ (alkyl), ═NR^{C}, spiro cycloalkyl group, fused cycloalkyl group or a monocyclic non-aromatic nitrogen-containing heterocyclic group attached by a ring nitrogen atom (e.g., N-piperidinyl, N-pyrrolidinyl, N-azepanyl, N-morpholinyl, N-thiomorphinyl, N-piperazinyl or N-diazepanyl group). Each R^{C} is independently selected from hydrogen, an unsubstituted alkyl group or a substituted alkyl group. Examples of substituents on the alkyl group represented by R* include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl. Preferred substituents for an alkyl or a ring carbon of a non-aromatic heterocyclic group include C1-C2 alkyl, —OH, N-pyrrolidinyl, N-piperidinyl, N-(4-alkyl)piperazinyl, N-morpholinyl or N-pyrrolyl.

A “substitutable ring atom” in a non-aromatic nitrogen-containing heterocyclic group is a ring carbon or nitrogen atom that is bonded to at least one hydrogen atom. The hydrogen atom can therefore optionally be replaced with the substituent. The term “substitutable ring atom” therefore excludes ring nitrogen and carbon atoms that already have three (for nitrogen) and four (for carbon) bonds to atoms other than hydrogen.

A preferred position for substitution of a non-aromatic nitrogen-containing heterocyclic group is the nitrogen ring atom. Suitable substituents on the nitrogen of a non-aromatic heterocyclic group or heteroaryl group include —R^{D}, —N(R^{D})₂, —C(O)R^{D}, —CO₂R^{D}, —C(O)C(O)R^{D}, —C(O)CH₂C(O)R^{D}, —SO₂R^{D}, —SO₂N(R^{D})₂, —C(═S)N(R^{D})₂, —C(═NH)—N(R^{D})₂, and —NR^{D}SO₂R^{D}; wherein R^{D} is hydrogen, an alkyl group, a substituted alkyl group, phenyl (Ph), substituted Ph, —O(Ph), substituted —OPh), CH₂(Ph), substituted CH₂(Ph), or an unsubstituted heteroaryl or heterocyclic ring. Examples of substituents on the alkyl group or the phenyl ring represented by Rˆ include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl. Preferred substituents on a substitutable nitrogen atom of a nitrogen-containing heteroaryl or nitrogen-containing non-aromatic heterocyclic group include C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy.

Additionally, pharmaceutically acceptable salts of the compounds of the disclosed angiogenesis inhibitors are included in the present invention. For example, an acid salt of a compound containing an amine or other basic group can be obtained, by reacting the compound with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, maleates, acetates, citrates, fumarates, tartrates [e.g. (+)-tartrates, (−)-tartrates or mixtures thereof including racemic mixtures], succinates, benzoates and salts with amino acids such as glutamic acid.

Salts of compounds containing a carboxylic acid or other acidic ftunctional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, N-benzyl- B-phenethylamine, dehydroabietylamine, N,N′-bisdehydroabietylamine, glucamine, N-methylglucamine, collidine, quinine, quinoline, and basic amino acid such as lysine and arginine.

“Treatment” or “treating” refers to both therapeutic and prophylactic treatment.

An “effective amount” is the quantity of an angiogenesis inhibitor in which a beneficial clinical outcome (prophylactic or therapeutic) is achieved when the compound is administered to a subject in need of treatment. For the treatment of rheumatoid arthritis or psoriasis, a “beneficial clinical outcome” includes a reduction in the severity of the symptoms associated with the disease (e.g., pain and inflammation), and/or a delay in the onset of the symptoms associated with the disease compared with the absence of the treatment. For the treatment of cancer, a beneficial clinical outcome includes a reduction in tumor mass, a reduction in the rate of tumor growth, a reduction in metastasis, a reduction in the severity of the symptoms associated with the cancer and/or an increase in the longevity of the subject compared with the absence of the treatment. For ocular diseases, a “beneficial clinical outcome” includes a reduction in the formation of abnormal blood vessels in the eye and the leakage and symptoms associated therewith. The precise amount of angiogenesis inhibitor administered to a subject will depend on the type and severity of the disease or condition and on the characteristics of the subject, such as general health, age, sex, body weight and tolerance to drugs. It will also depend on the degree, severity and type of disease or condition. The skilled artisan will be able to determine appropriate dosages depending on these and other factors. Effective amounts of the disclosed compounds typically range between about 0.1 mg/kg body weight per day and about 1000 mg/kg body weight per day, and preferably between 1 mg/kg body weight per day and 100 mg/kg body weight per day.

The angiogenesis inhibitors described herein, and the pharmaceutically acceptable salts, solvates and hydrates thereof can be used in pharmaceutical preparations in combination with a pharmaceutically acceptable carrier or diluent. Suitable pharmaceutically acceptable carriers include inert solid fillers or diluents and sterile aqueous or organic solutions. The angiogenesis inhibitor will be present in such pharmaceutical compositions in amounts sufficient to provide the desired dosage amount in the range described herein. Techniques for formulation and administration of the compounds of the instant invention can be found in Remington: the Science and Practice of Pharmacy, 19^{th} edition, Mack Publishing Co., Easton, Pa. (1995).

For oral administration, the angiogenesis inhibitor or salts thereof can be combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch, alginic acid; a lubricant such as magnesium stearate; and a sweetening agent such as sucrose lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For parental administration the disclosed angiogenesis, or salts thereof can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms. Aqueous solutions with up to 20% hydroxypropylˆ-cyclodextrin are commonly used.

In addition to the formulations described previously, the compounds may also be formulated as a long acting formulation, such as a depot preparation. Such long acting formulations may be administered by implantation, or, for example, subcutaneously by intramuscular injection. Depot formulations may be prepared from synthetic hydrogels such as those disclosed in U.S. Pat. Nos. 5,410,016, 6,177,095 and 6,632,457, the entire teachings of which are incorporated herein by reference. In certain applications, long acting formulations are implanted locally at the site of manifestation of the disease, for example, near, on or proximal to the affected organ or tissue.

Preferably disclosed angiogenesis inhibitors or pharmaceutical formulations containing these compounds are in unit dosage form for administration to a mammal. The unit dosage form can be any unit dosage form known in the art including, for example, a capsule, an IV bag, a tablet, or a vial. The quantity of the angiogenesis inhibitor in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

One embodiment of the present invention is a compound of formula (I). The values and preferred values for each variable in formula (I) are provided below.

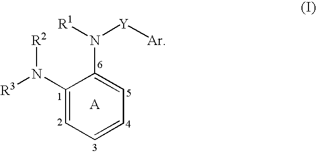

—Y— is —SO₂— or —C(O)—. Preferably, —Y— is —C(O)—.

R¹ is H or a C1-C6 alkyl group optionally substituted with amino, hydroxyl, methoxy or ethoxy. Preferably, R¹ is —H or C1-C3 alkyl.

R² and R³ are independently C1-C6 alkyl optionally substituted with amino, hydroxyl, methoxy or ethoxy. Preferably, R² and R³ are a C1-C4 alkyl group.

Alternatively, R₂ is absent and R₃, taken together with the nitrogen atom to which it is bonded and C1 and C2 of Ring A, forms an optionally substituted six membered nitrogen-containing aromatic group referred to as “Ring B”. Examples of suitable Ring B substituents are provided above in the section providing suitable aryl group substituents generally. Preferably, Ring B is represented by the following structural formula: 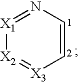
wherein carbon atom 1 and carbon atom 2 of Ring A are shared with Ring B and are also designated with “1” and “2”. Values for X₁—X₃ are provided below.

X₁—X₃ are independently N, CH or CR⁶, provided that X₁ and X₂ are not both N. In one preferred embodiment, X₁—X₃ are CR⁶. In another preferred embodiment, X₁ is N and X₂—X₃ are CR⁶. In another preferred embodiment, X₂ is N and X₁ and X₃ are CR⁶. In another preferred embodiment, X₃ is N and X₁ and X₂ are CR⁶. In another preferred embodiment, X₁ and X₃ are N and X₂ is CR⁶. In another preferred embodiment, X₂ and X₃ are N and X₁ is CR⁶. Each R⁶ group is independently selected. Suitable values for R⁶ are provided below.

Ar is an optionally substituted monocyclic aryl group. Preferably, Ar is an optionally substituted monocyclic nitrogen-containing aromatic group. More preferably, Ar is an optionally substituted pyridyl group. Even more preferably, the monocyclic nitrogen-containing aromatic group represented by Ar is 2-pyridyl substituted with p groups represented by R⁷. Each substituent on Ar is independently selected. Suitable values for p and R⁷ are provided below.

Ring A is optionally substituted at any one or more substitutable ring carbon atoms. Ring A is preferably substituted with m groups represented by R⁸. Suitable values for m and R⁸ are provided below. Each substituent on Ring A is independently selected.

Each R⁶ is independently halogen, alkyl, haloalkyl, —OR²⁰, —O(haloalkyl), —SR²⁰, —NO₂, —CN, —N(R²¹)₂, —NR²¹C(O)R²⁰, —NR²¹CO₂R²², —N(R²¹)C(O)N(R²¹)₂, —C(O)R²⁰, —CO₂R²⁰, —C(O)N(R²¹)₂, —S(O)₂R²⁰, —SO₂N(R²¹)₂, —S(O)R²², —NR²¹SO₂N(R²¹)₂, —NR²¹SO₂R²², —Vₒ—N(R²¹)₂, —O—Vₒₒ—N(R²¹)₂, —S—Vₒₒ—N(R²¹)₂ or —N(R²¹)—Vₒₒ—N(R²¹)₂. Preferably, each R⁶ is independently halogen, nitro, cyano, C1-C2 alkyl, C1-C2 haloalkyl, C1-C2 alkoxy, C1-C2 haloalkoxy, —O—(CH₂)₂—N(R²¹)₂, —CH₂—N(R²¹)₂ or —CH₂CH₂—N(R²¹)₂.

Each R⁷ is independently halogen, alkyl, haloalkyl, Ar¹, —OR³⁰, —O(haloalkyl), —SR³⁰, —NO₂, —CN, —N(R³¹)₂, —NR³¹C(O)R³⁰, —NR³¹CO₂R³², —N(R³¹)C(O)N(R³¹)₂, —C(O)R³⁰, —CO₂R³⁰, —S(O)₂R³⁰, —SO₂N(R³¹)₂, —S(O)R³², —NR³¹SO₂N(R³¹)₂, —NR³¹SO₂R³², —V—Ar¹, —V—OR³⁰, —V—O(haloalkyl), —V—SR³⁰, —V—NO₂, —V—CN, —V—N(R³¹)₂, —V—NR³¹C(O)R³⁰, —V—NR³¹CO₂R³², —V—N(R³¹)C(O)N(R³¹)₂, —V—C(O)R³⁰, —V—CO₂R³⁰, —V—S(O)₂R³⁰, —V—SO₂N(R³¹)₂, —V—S(O)R³², —V—NR³¹SO₂N(R³¹)₂, —V—NR³¹SO₂R³², —O—V—Ar¹, —O—V₁—N(R³¹)₂, —S—V—Ar¹, —S—V₁—N(R³¹)₂, —N(R³¹)—V₁—Ar¹, —N(R³¹)—V₁—N(R³¹)₂, —NR³¹C(O)—V—N(R³¹)₂, —NR³¹C(O)—V—Ar¹, —C(O)—V—N(R³¹)₂, —C(O)—V—Ar¹, —CO₂—V₁—N(R³¹)₂, —CO₂—V—Ar¹, —C(O)N(R³¹)—V₁—N(R³¹)₂, —C(O)N(R³¹)—V—Ar¹, —S(O)₂—V—N(R³¹)₂, —S(O)₂—V—Ar¹, —SO₂N(R³¹)—V₁—N(R³¹)₂, —SO₂N(R³¹)—V—Ar¹, —S(O)—V—N(R³¹)₂, —S(O)—V—Ar¹, —NR³¹SO₂—V—N(R³¹)₂ or —NR³¹SO₂—V—Ar¹; or two adjacent R⁷ groups, taken together, form a methylenedioxy, ethylenredioxy or —[CH₂]₄— group. Preferably, each R⁷ is independently selected from halogen, C1-C4 alkyl, C1-C4 haloalkyl, cyano, —OR³⁰, —SR³⁰, —N(R³¹)₂, Ar¹, —V—OR³⁰, —V—N(R⁴¹)₂, —V—Ar¹, —O—V—Ar¹, —O—V₁—N(R³¹)₂, —S—V—Ar¹, —S—V₁—N(R³¹)₂, —N(R³¹)—V—Ar², —N(R³¹)—V—Ar¹ or —N(R³¹)—V₁—N(R³¹)₂.

Each R⁸ is independently halogen, alkyl, haloalkyl, Ar², —OR⁴⁰, —O(haloalkyl), —SR⁴⁰, —NO₂, —CN, —N(R⁴¹)₂, —NR⁴¹C(O)R⁴⁰, —NR⁴¹CO₂R⁴², —N(R⁴¹)C(O)N(R⁴¹)₂, —C(O)R⁴⁰, —CO₂R⁴⁰, —C(O)N(R⁴¹)₂, —S(O)₂R⁴⁰, —SO₂N(R⁴¹)₂, —S(O)R⁴², —NR⁴¹SO₂N(R⁴¹)₂, —NR⁴¹SO₂R⁴², —V₂—Ar², —V₂—OR⁴⁰, —V₂—O(haloalkyl), —V₂—SR⁴⁰, —V₂—NO₂, —V₂—CN, —V₂—N(R⁴¹)₂, —V₂—NR⁴¹C(O)R⁴⁰, —V₂—NR⁴¹CO₂R⁴², —V₂—N(R⁴¹)C(O)N(R⁴¹)₂, —V₂—C(O)R⁴⁰, —V₂—CO₂R⁴⁰, —V₂—C(O)N(R⁴¹)₂, —V₂S(O)₂R⁴⁰, —V₂—SO₂N(R⁴¹)₂, —V₂—S(O)R⁴², —V₂—NR⁴¹SO₂N(R⁴¹)₂, —V₂—NR⁴¹SO₂R⁴², —O—V₂—Ar², —O—V₃—N(R⁴¹)₂, —S—V₂—Ar², —S—V₃—N(R⁴¹)₂, —N(R⁴¹)—V₂—Ar², —N(R⁴¹)—V₃——N(R⁴¹)₂, —NR⁴¹C(O)—V₂—N(R⁴¹)₂, —NR⁴¹C(O)—V₂—Ar², —C(O)—V₂—N(R⁴¹)₂, —C(O)—V₂—Ar², —CO₂—V₂—N(R⁴¹)₂, —CO₂—V₂—Ar², —C(O)N(R⁴¹)—V₃—N(R⁴¹)₂, —C(O)N(R⁴¹)—V₂—Ar², —S(O)₂—V₂—N(R⁴¹)₂, —S(O)₂—V₂—Ar², —SO₂N(R⁴¹)—V₃—N(R⁴¹)₂, —SO₂N(R⁴¹)—V₂—Ar², —S(O)—V₂—N(R⁴¹)₂, —S(O)—V₂—Ar², —NR⁴¹SO₂—V₂—N(R⁴¹)₂ or —NR⁴¹SO₂—V₂—Ar²; or two adjacent R⁸ groups, taken together, form a methylenedioxy, ethylenedioxy or —[CH₂]₄— group. Preferably, each R⁸ is independently selected from halogen, C1-C4 alkyl, C1-C4 haloalkyl, cyano, —OR⁴⁰, —SR⁴⁰, —N(R⁴¹)₂, Ar², —V₂—OR⁴⁰, —V₂—N(R⁴¹)₂, —V₂—Ar², —O—V₂—Ar², —O—V₃—N(R⁴¹)₂, —S—V₂—Ar², —S—V₃—N(R⁴¹)₂, —N(R⁴¹)—V₂—Ar² or —N(R⁴¹)—V₃—N(R⁴¹)₂.

Each R²⁰ is independently hydrogen or an alkyl group.

Each R²¹ is independently hydrogen, an alkyl group, —CO₂R²⁰, —SO₂R²⁰ or —C(O)R²⁰; or —N(R²¹)₂ is an optionally substituted non-aromatic nitrogen-containing heterocyclic group. Suitable substituents for the non-aromatic nitrogen-containing heterocyclic group represented by —N(R²¹)₂ is provided in section describing suitable non-aromatic heterocyclic group substituents generally. Preferably, each R²¹ is independently —H or an alkyl group or —N(R²¹)₂ taken together is a five or six-membered non-aromatic nitrogen-containing heterocyclic group.

Each R²² is independently an alkyl group.

Each R³⁰ is independently: i) hydrogen; ii) an aromatic group substituted with zero, one or two groups represented by halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy. Preferably, each R³⁰ is independently: i) hydrogen; ii) a phenyl group substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R³¹ is independently R³⁰, —CO₂R³⁰, —SO₂R³⁰ or —C(O)R³⁰; or —N(R³¹)₂ taken together is an optionally substituted non-aromatic heterocyclic group. Preferably, each R³¹ is independently R³⁰, or —N(R³¹)₂ is an optionally substituted non-aromatic heterocyclic group. More preferably, each R³¹ is independently R³⁰; or —N(R³¹)₂ taken together is an optionally substituted N-piperidinyl, N-pyrrolidinyl, N-azepanyl, N-morpholinyl, N-thiomorphinyl, N′-substituted-N-piperazinyl or N′-substituted-N-diazepanyl group. Suitable substitutents for a non-aromatic heterocyclic ring represented by —N(R³¹)₂ are described above in the section providing suitable substitutents for a non-aromatic heterocyclic group generally. Preferred substitutents for a substitutable ring carbon of a monocyclic non-aromatic heterocyclic group represented by —N(R³¹)₂ are C1-C2 alkyl, —OH, N-pyrrolidinyl, N-piperidinyl, N-(4-alkyl)piperazinyl, N-morpholinyl or N-pyrrolyl; and preferred substituents for a substitutable ring nitrogen atom of a monocyclic non-aromatic heterocyclic group represented by —N(R³¹)₂ are C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy. Each ring carbon and ring nitrogen substituents is independently selected.

Each R³² is independently: i) an aromatic group substituted with zero, one or two groups represented by halogen, alkyl, amino, alkylamino, dialkylamino, alkoxy, nitro, cyano, hydroxy, haloalkoxy or haloalkyl; or ii) an alkyl group optionally substituted with halogen, hydroxyl, alkoxy, nitro, cyano, alkoxycarbonyl, alkylcarbonyl or haloalkoxy.

Each R⁴⁰ is independently: i) hydrogen; ii) an aromatic substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy. Preferably, each R⁴⁰ is independently: i) hydrogen; ii) a phenyl group substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R⁴¹ is independently R⁴⁰, —CO₂R⁴⁰, —SO₂R⁴⁰ or —C(O)R⁴⁰; or —N(R⁴¹)₂ taken together is an optionally substituted non-aromatic heterocyclic group. Preferably, each R⁴¹ is independently R⁴⁰, or —N(R⁴¹)₂ is an optionally substituted non-aromatic heterocyclic group. Preferably, each R⁴¹ is independently R⁴⁰, or —N(R⁴¹)₂ is an optionally substituted non-aromatic heterocyclic group. More preferably, each R⁴¹ is independently R⁴⁰; or —N(R⁴¹)₂ taken together is an optionally substituted N-piperidinyl, N-pyrrolidinyl, N-azepanyl, N-morpholinyl, N-thiomorphinyl, N-piperazinyl or N-diazepanyl. Suitable substitutents for a non-aromatic heterocyclic ring represented by —N(R⁴¹)² are described above in the section providing suitable substitutents for a non-aromatic heterocyclic group generally. Preferred substituents for a substitutable ring carbon of a monocyclic non-aromatic heterocyclic group represented by —N(R⁴¹)₂ are C1-C2 alkyl, —OH, N-pyrrolidinyl, N-piperidinyl, N-(4-alkyl)piperazinyl, N-morpholinyl or N-pyrrolyl; and preferred substituents for a substitutable ring nitrogen atom of a monocyclic non-aromatic heterocyclic group represented by —N(R⁴¹)₂ are C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy. Each ring carbon and ring nitrogen substituents is independently selected.

Each R⁴² is independently: i) an aromatic substituted with zero, one or two groups independently selected from halogen, alkyl, amino, alkylamino, dialkylamino, alkoxy, nitro, cyano, hydroxy, haloalkoxy or haloalkyl; or ii) an alkyl group optionally substituted with halogen, hydroxyl, alkoxy, nitro, cyano, alkoxycarbonyl, alkylcarbonyl or haloalkoxy.

Ar¹ and Ar² are independently a monocyclic aromatic group (preferably a phenyl group) each substituted with zero, one or two groups independently selected from halogen, alkyl, amino, alkylamino, dialkylamino, alkoxy, nitro, cyano, hydroxy, haloalkoxy or haloalkyl.

Each Vₒ is independently a C1-C4 alkylene group.

Each Vₒₒ is independently a C2-C4 alkylene group.

Each V is independently a C1-C4 alkylene group.

Each V₁ is independently a C2-C4 alkylene group.

Each V₂ is independently a C1-C4 alkylene group.

Each V₃ is independently a C2-C4 alkylene group.

m is 0, 1, 2, 3 or 4; preferably 0, 1 or 2.

p is 0, 1, 2, 3 or 4; preferably 0, 1 or 2.

In certain instances, at least one of Ring A, Ring B and Ar is substituted with a group other than alkyl when Ar is pyridyl; and Ar is not substituted with a carboxamide when Ar is phenyl or pyridyl. In these instances, the term “carboxamide” is understood to mean —C(O)NR₂ wherein R is an optionally substituted alkyl or aryl group.

In a preferred embodiment of the present invention, the angiogenesis inhibitor is represented by Structural Formula (III): 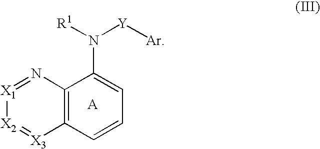
Values and preferred values for the variables in Structural Formula (III) are as described above for Structural Formula (I). In a more preferred embodiment, the values for the variables in Structural Formula (III) are described in the paragraphs below.

Ring A is optionally substituted at any one or more substitutable ring atoms.

X₁—X₃ are independently N, CH or CR⁶, provided that X₁ and X₂ are not both N.

Each R⁶ is independently halogen, alkyl, haloalkyl, —OR²⁰, —O(haloalkyl), —SR²⁰, —NO₂, —CN, —N(R²¹)₂, —NR²¹C(O)R²⁰, —NR²¹CO₂R²², —N(R²¹)C(O)N(R²¹)₂, —C(O)R²⁰, —CO₂R²⁰, —C(O)N(R²¹)₂, —S(O)₂R²⁰, —SO₂N(R²¹)₂, —S(O)R²², —NR²¹SO₂N(R²¹)₂, —NR²¹SO₂R²², —Vₒ—N(R²¹)₂, —O—Vₒₒ—N(R²¹)₂, —S—Vₒₒ—N(R²¹)₂ or —N(R²¹)—Vₒₒ—N(R²¹)₂.

Each Vₒ is independently a C1-C4 alkylene group.

Each Vₒₒ is independently a C2-C4 alkylene group.

Each R²⁰ is independently hydrogen or an alkyl group.

Each R²¹ is independently hydrogen, an alkyl group, —CO₂R²⁰, —SO₂R²⁰ or —C(O)R²⁰; or —N(R²¹)₂ is an optionally substituted non-aromatic nitrogen-containing heterocyclic group.

Each R²² is independently an alkyl group.

The remainder of the variables in Structural Formula (III) are as defined for Structural Formula (I). Preferably, Ar is an optionally substituted monocyclic nitrogen-containing aromatic group, preferably an optionally substituted pyridyl group.

In another preferred embodiment, the disclosed angiogenesis inhibitor is represented by Structural Formulas (IV): 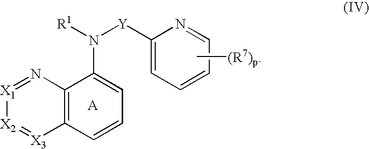
Values and preferred values for the variables in Structural Formula (IV) are as described above for Structural Formula (II). In a more preferred embodiment, the variables in Structural Formula (IV) are as described in the paragraphs below.

Ring A is optionally substituted at any one or more substitutable rings carbon atoms.

p is 0, 1, 2, 3 or 4.

Each R⁷ is independently halogen, alkyl, haloalkyl, Ar¹, —OR³⁰, —O(haloalkyl), —SR³⁰, —NO₂, —CN, —N(R³¹)₂, —NR³¹C(O)R³⁰, —NR³⁰CO₂R³², —N(R³¹)C(O)N(R³¹)₂, —C(O)R³⁰, —CO₂R³⁰, —S(O)₂R³⁰, —SO₂N(R³¹)₂, —S(O)R³², —NR³¹SO₂N(R³¹)₂, —NR³¹SO₂R³², —V—Ar¹, —V—OR³⁰, —V—O(haloalkyl), —V—SR³⁰, —V—NO₂, —V—CN, —V—N(R³¹)₂, —V—NR³¹C(O)R³⁰, —V—NR³¹CO₂R³², —V—N(R³¹)C(O)N(R³¹)₂, —V—C(O)R³⁰, —V—CO₂R³⁰, —V—S(O)₂R³⁰, —V—SO₂N(R³¹)₂, —V—S(O)R³², —V—NR³¹SO₂N(R³¹)₂, —V—NR³¹SO₂R³², —O—V—Ar¹, —O—V₁—N(R³¹)₂, —S—V—Ar¹, —S—V₁—N(R³¹)₂, —N(R³¹)—V₁—Ar¹, —N(R³¹)—V₁—N(R³¹)₂, —NR³¹C(O)—V—N(R³¹)₂, —NR³¹C(O)—V—Ar¹, —C(O)—V—N(R³¹)₂, —C(O)—V—Ar¹, —CO₂—V₁—N(R³¹)₂, —CO₂—V—Ar¹, —C(O)N(R³¹)—V₁—N(R³¹)₂, —C(O)N(R³¹)—V—Ar¹, —S(O)₂—V—N(R³¹)₂, —S(O)₂—V—Ar¹, —SO₂N(R³¹)—V₁—N(R³¹)₂, —SO₂N(R³¹)—V—Ar¹, —S(O)—V—N(R³¹)₂, —S(O)—V—Ar¹, —NR³¹SO₂—V—N(R³¹)₂ or —NR³¹SO₂—V—Ar¹; or two adjacent R⁷ groups, taken together, form a methylenedioxy, ethylenedioxy or —[CH₂]₄— group.

Each V is independently a C1-C4 alkylene group.

Each V₁ is independently a C2-C4 alkylene group.

Ar¹ is a monocyclic aromatic group each substituted with zero, one or two groups independently selected from halogen, alkyl, amino, alkylamino, dialkylamino, alkoxy, nitro, cyano, hydroxy, haloalkoxy or haloalkyl.

Each R³⁰ is independently: i) hydrogen; ii) an aromatic group substituted with zero, one or-two groups represented by halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R³is independently R³⁰, —CO₂R³⁰, —SO₂R³⁰ or —C(O)R³⁰; or —N(R³¹)₂ taken together is an optionally substituted non-aromatic heterocyclic group.

Each R³² is independently: i) an aromatic group substituted with zero, one or two groups represented by halogen, alkyl, amino, alkylamino, dialkylamino, alkoxy, nitro, cyano, hydroxy, haloalkoxy or haloalkyl; or ii) an alkyl group optionally substituted with halogen, hydroxyl, alkoxy, nitro, cyano, alkoxycarbonyl, alkylcarbonyl or haloalkoxy.

The values and preferred values for the remainder of the variables in Structural Formula (IV) are as described above for Structural Formula (II).

In another preferred embodiment, the disclosed angiogenesis inhibitor is represented by Structural Formulas (V), (VI) or (VII):

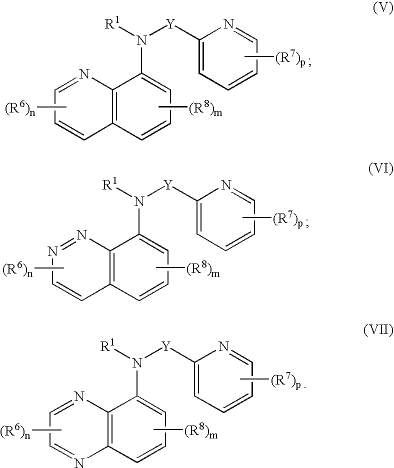

The values and preferred values for the variables in Structural Formulas (V), (VI) and (VII) are as described for Structural Formula (I). In a first more preferred embodiment, the variables in Structural Formulas (IV), (VI) and (VII) are described immediately below.

In Structural Formula (V), n and m are independently 0, 1, 2 or 3; and in Structural Formulas (VI) and (VII), n is 0, 1 or 2 and m is 0, 1, 2 or 3.

Each R⁸ is independently: i) halogen, alkyl, haloalkyl, Ar², —OR⁴⁰, —O(haloalkyl), —SR⁴⁰, —NO₂, —CN, —N(R⁴¹)₂, —NR⁴¹C(O)R⁴⁰, —NR⁴¹CO₂R⁴², —N(R⁴¹)C(O)N(R⁴¹)₂, —C(O)R⁴⁰, —CO₂R⁴⁰, —C(O)N(R⁴¹)₂, —S(O)₂R⁴⁰, —SO₂N(R⁴¹)₂, —S(O)R⁴², —NR⁴¹SO₂N(R⁴¹)₂, —NR⁴¹SO₂R⁴², —V₂—Ar², —V₂—OR⁴⁰, —V₂—O(haloalkyl), —V₂—SR⁴⁰, —V₂—NO₂, —V₂—CN, —V₂—N(R⁴¹)₂, —V₂—NR⁴¹C(O)R⁴⁰, —V₂—NR⁴¹CO₂R⁴², —V₂—N(R⁴¹)C(O)N(R⁴¹)₂, —V₂—C(O)R⁴⁰, —V₂—CO₂R⁴⁰, —V₂—C(O)N(R⁴¹)₂, —V₂—S(O)₂R⁴⁰, —V₂—SO₂N(R⁴¹)₂, —V₂—S(O)R⁴², —V₂—NR⁴¹SO₂N(R⁴¹)₂, —V₂—NR⁴¹SO₂R⁴², —O—V₂—Ar², —O—V₃—N(R⁴¹)₂, —S—V₂—Ar², —S—V₃—N(R⁴¹)₂, —N(R⁴¹)—V₂—Ar², —N(R⁴¹)—V₃—N(R⁴¹)₂, —NR⁴¹C(O)—V₂—N(R⁴¹)₂, —NR⁴¹C(O)—V₂—Ar², —C(O)—V₂—N(R⁴¹)₂, —C(O)—V₂—Ar², —CO₂—V₂—N(R⁴¹)₂, —CO₂—V₂—Ar², —C(O)N(R⁴¹)—V₃—N(R⁴¹)₂, —C(O)N(R⁴¹)—V₂—Ar², —S(O)₂—V₂—N(R⁴¹)₂, —S(O)₂—V₂—Ar , —SO₂N(R⁴¹)—V₃—N(R⁴¹)₂, —SO₂N(R⁴¹)—V₂—Ar², —S(O)—V₂—N(R⁴¹)₂, —S(O)—V₂—Ar², —NR⁴¹SO₂—V₂—N(R⁴¹)₂ or —NR⁴¹SO₂—V₂Ar²; or ii) two adjacent R⁸ groups, taken together, form a methylenedioxy, ethylenedioxy or —[CH₂]₄— group.

Each V₂ is independently a C1-C4 alkylene group.

Each V₃ is independently a C2-C4 alkylene group.

Ar² is a monocyclic aromatic group substituted with zero, one or two groups independently selected from halogen, alkyl, amino, alkylamino, dialkylamino, alkoxy, nitro, cyano, hydroxy, haloalkoxy or haloalkyl.

Each R⁴⁰ is independently: i) hydrogen; ii) an aromatic substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R⁴¹ is independently R⁴⁰, —CO₂R⁴⁰, —SO₂R⁴⁰ or —C(O)R⁴⁰; or —N(R⁴¹)₂ taken together is an optionally substituted non-aromatic heterocyclic group.

Each R⁴² is independently: i) an aromatic substituted with zero, one or two groups independently selected from halogen, alkyl, amino, alkylamino, dialkylamino, alkoxy, nitro, cyano, hydroxy, haloalkoxy or haloalkyl; or ii) an alkyl group optionally substituted with halogen, hydroxyl, alkoxy, nitro, cyano, alkoxycarbonyl, alkylcarbonyl or haloalkoxy.

The values and preferred values for the remainder of the variables in Structural Formulas (V), (VI) and (VII) are as described for Structural Formula (IV) above.

In a second more preferred embodiment, the variables in Structural Formulas (V), (VI) and (VII) are described in the paragraphs immediately below.

Each R⁷ is independently selected from halogen, C1-C4 alkyl, C1-C4 haloalkyl, cyano, —OR³⁰, —SR³⁰, —N(R³¹)₂, Ar¹, —V—OR³⁰, —V—N(R⁴¹)₂, —V—Ar¹, —O—V—Ar¹, —O—V₁—N(R³¹)₂, —S—V—Ar¹, —S—V₁—N(R³¹)₂, —N(R³¹)—V—Ar¹ or —N(R³¹)—V₁—N(R³¹)₂;

Each R⁸ is independently selected from halogen, C1-C4 alkyl, C1-C4 haloalkyl, cyano, —OR⁴⁰, —SR⁴⁰, —N(R⁴¹)₂, Ar², —V₂—OR⁴⁰, —V₂—N(R⁴¹)₂, —V₂—Ar², —O—V₂—Ar², —O—V₃—N(R⁴¹)₂, —S—V₂—Ar², —S—V₃—N(R⁴¹)₂, —N(R⁴¹)—V₂—Ar² or —N(R⁴¹)—V₃—N(R⁴¹)₂.

Ar¹ and Ar² are phenyl, each independently substituted with zero, one or two groups independently selected from halogen, alkyl, amino, alkylamino, dialkylamino, alkoxy, nitro, cyano, hydroxy, haloalkoxy and haloalkyl.

each R³⁰ and each R⁴⁰ is independently: i) hydrogen; ii) a phenyl group substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R³¹ is independently R³⁰, or —N(R³¹)₂ is an optionally substituted non-aromatic heterocyclic group.

Each R⁴¹ is independently R⁴⁰, or —N(R⁴¹)₂ is an optionally substituted non-aromatic heterocyclic group.

The values and preferred values for the remainder of the variables are as described in the first more preferred embodiment for Structural Formulas (V), (VI) and (VII).

In a third more preferred embodiment, the values for the variables in Structural Formulas (V), (VI) and (VII) are described in the paragraphs immediately below.

Each R⁶ is independently halogen, nitro, cyano, C1-C2 alkyl, C1-C2 haloalkyl, C1-C2 alkoxy, C1-C2 haloalkoxy, —O—(CH₂)₂—N(R²¹)₂, —CH₂—N(R²¹)₂ or —CH₂CH₂—N(R²¹)₂.

Each R²¹ is independently —H or an alkyl group or —N(R²¹)₂ taken together is a five or six-membered non-aromatic nitrogen-containing heterocyclic group.

The values for the remainder of the variables are as described for the second more preferred embodiment for Structural Formulas (V), (VI) and (VII).

In another preferred embodiment, the angiogenesis inhibitor of the present invention in represented by Structural Formula (VIII), (IX) or (X):

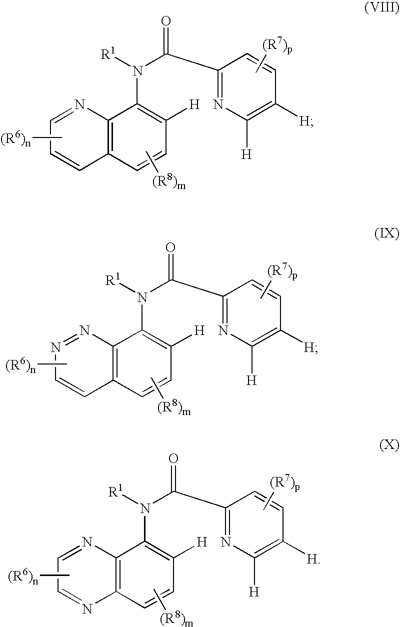

The values and preferred values for the variables in Structural Formulas (VIII), (IX) and (X) are as described for Structural Formula (I). In a first more preferred embodiment, the values for the variables in Structural Formulas (VIII), (IX) and (X) are described immediately below:

n, m and p are independently 0, 1, or 2.

R¹ is H or a C1-C6 alkyl group optionally substituted with amino, hydroxyl, methoxy or ethoxy.

Each R⁶ is independently halogen, nitro, cyano, C1-C2 alkyl, C1-C2 haloalkyl, C1-C2 alkoxy, C1-C2 haloalkoxy, —O—(CH₂)₂—N(R²¹)₂, —CH₂—N(R²¹)₂ or —CH₂CH₂—N(R²¹)₂.

Each R⁷ is independently selected from halogen, C1-C4 alkyl, C1-C4 haloalkyl, cyano, —OR³⁰, —SR³⁰, —N(R³¹)₂, Ar¹, —V—OR³⁰, —V—N(R⁴¹)₂, —V—Ar¹, —O—V—Ar¹, —O—V₁—N(R³¹)₂, —S—V—Ar¹, —S—V₁—N(R³¹)₂, —N(R³¹)—V₁—Ar¹ or —N(R³¹)₂,

Each R⁸ is independently selected from halogen, C1-C4 alkyl, C1-C4 haloalkyl, cyano, —OR⁴⁰, —SR⁴⁰, —N(R⁴¹)₂, Ar², —V₂—OR⁴⁰, —V₂—N(R⁴¹)₂, —V₂—Ar², —O—V₂—Ar², —O—V₃—N(R⁴¹)₂, —S—V₂—Ar², —S—V₃—N(R⁴¹)₂, —N(R⁴¹)—V₂—Ar² or —N(R⁴¹)—V₃—N(R⁴¹)₂.

Ar¹ and Ar² are phenyl, each independently substituted with zero, one or two groups independently selected from halogen, alkyl, amino, alkylamino, dialkylamino, alkoxy, nitro, cyano, hydroxy, haloalkoxy and haloalkyl.

Each V and each V₂ is independently a C1-C4 alkylene group.

Each V₁ and each V₃ is independently a C2-C4 alkylene group.

Each R²¹ is independently —H or an alkyl group or —N(R²¹)₂ taken together is a five or six-membered non-aromatic nitrogen-containing heterocyclic group.

Each R³⁰ and each R⁴⁰ is independently: i) hydrogen; ii) a phenyl group substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R³¹ is independently R³⁰; or —N(R³¹)₂ taken together is a monocyclic non-aromatic heterocyclic group, wherein: i) the monocyclic non-aromatic heterocyclic group represented by —N(R³¹)₂ is optionally and independently substituted at any one or more substitutable ring carbon with C1-C2 alkyl, —OH, N-pyrrolidinyl, N-piperidinyl, N-(4-alkyl)piperazinyl, N-morpholinyl or N-pyrroly; and ii) the monocyclic non-aromatic heterocyclic group represented by —N(R³¹)₂ is optionally and independently substituted at any substitutable ring nitrogen atom with C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy.

Each R⁴¹ is independently R⁴⁰; or —N(R⁴¹)₂ is a monocyclic non-aromatic heterocyclic group, wherein: i) the monocyclic non-aromatic heterocyclic group represented by —N(R⁴¹)₂ is optionally and independently substituted at any one or more substitutable ring carbon with C1-C2 alkyl, —OH, N-pyrrolidinyl, N-piperidinyl, N-(4-alkyl)piperazinyl, N-morpholinyl or N-pyrrolyl; or ii) the monocyclic non-aromatic heterocyclic group represented by —N(R⁴¹)₂ is optionally and independently substituted at any substitutable ring nitrogen atom with C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy.

In a second more preferred embodiment, the values for the variables in Structural Formulas (VIII), (IX) and (X) are described in the paragraphs immediately below.

Each R³¹ is independently R³⁰; or —N(R³¹)₂ taken together is an optionally substituted N-piperidinyl, N-pyrrolidinyl, N-azepanyl, N-morpholinyl, N-thiomorphinyl, N′-substituted-N-piperazinyl or N′-substituted-N-diazepanyl group.

Each R⁴¹ is independently R⁴⁰; or —N(R⁴¹)₂ taken together is an optionally substituted N-piperidinyl, N-pyrrolidinyl, N-azepanyl, N-morpholinyl, N-thiomorphinyl, N-piperazinyl or N-diazepanyl.

The remainder of the variables are as described for the first more preferred embodiment for Structural Formulas (VIII), (IX) and (X).

In another preferred embodiment, the angiogenesis inhibitor of the invention is represented by Structural Formula (XI) or (XII):

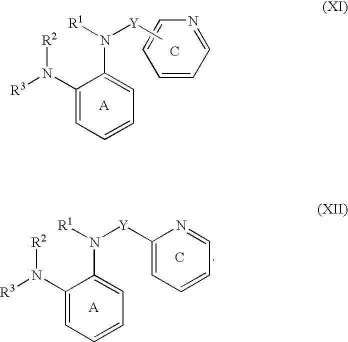

R² and R³ in Structural Formulas (XI) and (XII) are independently C1-C6 alkyl optionally substituted with amino, hydroxyl, methoxy or ethoxy. The values and preferred values for the remainder of the variables in Structural Formulas (XI) and (XII) are as described above for Structural Formula (I). In a more preferred embodiment, the values for the variables in Structural Formulas (XI) and (XII) are described below:

Pyridyl Ring C is optionally substituted at any one or more substitutable ring carbon atoms and each Ring C substituent is independently selected.

R¹ is —H or C1-C3 alkyl.

R² and R³ are independently C1-C6 alkyl optionally substituted with amino, hydroxyl, methoxy or ethoxy.

The values and preferred values for the remainder of the variables are as described for Structural Formula (I).

In another preferred embodiment, the angiogenesis inhibitor of the present invention is represented by Structural Formula (XIII):

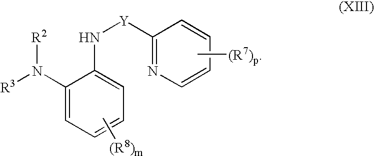

The values and preferred values for the variables in Structural Formula (XIII) are as described above for Structural Formulas (XI) and (XII). In a first more preferred embodiment, the values for the variables in Structural Formula (XIII) are described in the following paragraph:

m and p are independently 0, 1, 2, 3 or 4.

R² and R³ are independently C1-C6 alkyl optionally substituted with amino, hydroxyl, methoxy or ethoxy.

Each R⁷ is independently: i) halogen, alkyl, haloalkyl, Ar¹, —OR³⁰, —O(haloalkyl), —SR³⁰, —NO₂, —CN, —N(R³¹)₂, —NR³¹C(O)R³⁰, —NR³¹CO₂R³², —N(R³¹)C(O)N(R³¹)₂, —C(O)R³⁰, —CO₂R³⁰, —S(O)₂R³⁰, —SO₂N(R³¹)₂, —S(O)R³², —NR³¹SO₂N(R³¹)₂, —NR³¹SO₂R³², —V—Ar¹, —V—OR³⁰, —V—O(haloalkyl), —V—SR³⁰, —V—NO₂, —V—CN, —V—N(R³¹)₂, —V—NR³¹C(O)R³⁰, —V—NR³¹CO₂R³², —V—N(R³¹)C(O)N(R³¹)₂, —V—C(O)R³⁰, —V—CO₂R³⁰, —V—S(O)₂R³⁰, —V—SO₂N(R³¹)₂, —V—S(O)R³², —V—NR³¹SO₂N(R³¹)₂, —V—NR³¹SO₂R³², —O—V—Ar¹, —O—V₁—N(R³¹)₂, —S—V—Ar¹, —S—V₁—N(R³¹)₂, —N(R³¹)—V—Ar¹, —N(R³¹)—V₁—N(R³¹)₂, —NR³¹C(O)—V—N(R³¹)₂, —NR³¹C(O)—V—Ar¹, —C(O)—V—N(R³¹)₂, —C(O)—V—Ar¹, —CO₂—V₁—N(R³¹)₂, —CO₂—V—Ar¹, —C(O)N(R³¹)—V₁—N(R³¹)₂, —C(O)N(R³¹)—V—Ar¹, —S(O)₂—V—N(R³¹)₂, —S(O)₂—V—Ar¹, —SO₂N(R³¹)—V₁—N(R³¹)₂, —SO₂N(R³¹)—V—Ar¹, —S(O)—V—N(R³¹)₂, —S(O)—V—Ar¹, —NR³¹ SO₂—V—N(R³¹)₂ or —NR³¹SO₂—V—Ar¹; or ii) two adjacent R⁷ groups, taken together, form a methylenedioxy, ethylenedioxy or —[CH₂]₄— group.

Each R⁸ is independently: i) halogen, alkyl, haloalkyl, Ar², —OR⁴⁰, —O(haloalkyl), —SR⁴⁰, —NO₂, —CN, —N(R⁴¹)₂, —NR⁴¹C(O)R⁴⁰, —NR⁴¹CO₂R⁴², —N(R⁴¹)C(O)N(R⁴¹)₂, —C(O)R⁴⁰, —CO₂R⁴⁰, —C(O)N(R⁴¹)₂, —S(O)₂R⁴⁰, —SO₂N(R⁴¹)₂, —S(O)R⁴², —NR⁴¹SO₂N(R⁴¹)₂, —NR⁴¹SO₂R⁴², —V₂—Ar², —V₂—OR⁴⁰, —V₂—O(haloalkyl), —V₂—SR⁴⁰, —V₂—NO₂, —V₂—CN, —V₂—N(R⁴¹)₂, —V₂—NR₄₁C(O)R⁴⁰, —V₂—NR⁴¹CO₂R⁴², —V₂—N(R⁴¹)C(O)N(R⁴¹)₂, —V₂—C(O)R⁴⁰, —V₂—CO₂R⁴⁰, —V₂—C(O)N(R⁴¹)₂, —V₂—S(O)₂R⁴⁰, —V₂—SO₂N(R⁴¹)₂, —V₂—S(O)R⁴², —V₂—NR⁴¹SO₂N(R⁴¹)₂, —V₂—NR⁴¹SO₂R⁴², —O—V₂—Ar², —O—V₃—N(R⁴¹)₂, —S—V₂—Ar², —S—V₃—N(R⁴¹)₂, —N(R⁴¹)—V₂—Ar², —N(R⁴¹)—V₃—N(R⁴¹)₂, —NR⁴¹C(O)—V₂—N(R⁴¹)₂, —NR⁴¹C(O)—V₂—Ar², —C(O)—V₂—N(R⁴¹)₂, —C(O)—V₂—Ar², —CO₂—V₃—N(R⁴¹)₂, —CO₂—V₂—Ar², —C(O)N(R⁴¹)—V₃—N(R⁴¹)₂, —C(O)N(R⁴¹)—V₂—Ar², —S(O)₂—V₂—N(R⁴¹)₂, —S(O)₂—V₂—Ar², —SO₂N(R⁴¹)—V₃—N(R⁴¹)₂, —SO₂N(R⁴¹)—V₂—Ar², —S(O)—V₂—N(R⁴¹)₂, —S(O)—V₂—Ar², —NR⁴¹SO₂—V₂—N(R⁴¹)₂ or —NR⁴¹SO₂—V₂—Ar²; or ii) two adjacent R⁸ groups, taken together, form a methylenedioxy, ethylenedioxy or —[CH₂]₄— group.

Each V and each V₂ is independently a C1-C4 alkylene group.

Each V₁ and each V₃ is independently a C2-C4 alkylene group.

Ar¹ and Ar² are each independently a monocyclic aromatic group substituted with zero, one or two groups independently selected from halogen, alkyl, amino, alkylamino, dialkylamino, alkoxy, nitro, cyano, hydroxy, haloalkoxy or haloalkyl.

Each R³⁰ and each R⁴⁰ is independently: i) hydrogen; ii) an aromatic group substituted with zero, one or two groups represented by halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R³¹ is independently R³⁰, —CO₂R³⁰, —SO₂R³⁰ or —C(O)R³⁰; or —N(R³¹)₂ taken together is an optionally substituted non-aromatic heterocyclic group.

Each R⁴¹ is independently R⁴⁰, —CO₂R⁴⁰, —SO₂R⁴⁰ or —C(O)R⁴⁰; or —N(R⁴¹)₂ taken together s an optionally substituted non-aromatic heterocyclic group.

Each R³² and each R⁴² is independently: i) an aromatic group substituted with zero, one or two groups represented by halogen, alkyl, amino, alkylamino, dialkylamino, alkoxy, nitro, cyano, hydroxy, haloalkoxy or haloalkyl; or ii) an alkyl group optionally substituted with halogen, hydroxyl, alkoxy, nitro, cyano, alkoxycarbonyl, alkylcarbonyl or haloalkoxy.

The values and preferred values for the remainder of the variables are as described for Structural Formulas (XI) and (XII).

In a second more preferred embodiment, the variables in Structural Formula (XIII) are described below.

Each R⁷ is independently selected from halogen, C1-C4 alkyl, C1-C4 haloalkyl, cyano, —OR³⁰, —SR³⁰, —N(R³¹)₂, Ar¹, —V—OR³⁰, —V—N(R⁴¹)₂, —V—Ar¹, —O—V—Ar¹, —O—V₁—N(R³¹)₂, —S—V—Ar¹, —S—V₁—N(R³¹)₂, —N(R³¹)—V—Ar¹ or —N(R³¹)—V₁—N(R³¹)₂.

Each R⁸ is independently selected from halogen, C1-C4 alkyl, C1-C4 haloalkyl, cyano, —OR⁴⁰, —SR⁴⁰, —N(R⁴¹)₂, Ar², —V₂—OR⁴⁰, —V₂—N(R⁴¹)₂, —V₂—Ar², —O—V₃—Ar², —O—V₃—N(R⁴¹)₂, —S—V₂—Ar², —S—V₃—N(R⁴¹)₂, —N(R⁴¹)—V₂—Ar² or —N(R⁴¹)—V₃—N(R⁴¹)₂.

Ar¹ and Ar² are phenyl, each independently substituted with zero, one or two groups independently selected from halogen, alkyl, amino, alkylamino, dialkylamino, alkoxy, nitro, cyano, hydroxy, haloalkoxy or haloalkyl.

Each R³⁰ and each R⁴⁰ is independently: i) hydrogen; ii) a phenyl group substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R³¹ is independently R³⁰, or —N(R³¹)₂ taken together is an optionally substituted non-aromatic heterocyclic group.

Each R⁴¹ is independently R⁴⁰, or —N(R⁴¹)₂ taken together is an optionally substituted non-aromatic heterocyclic group.

The values and preferred values for the remainder of the variables are as described above for the first more preferred more embodiment for Structural Formula (XIII).

In another more preferred embodiment, the angiogenesis inhibitor of the invention is represented by Structural Formula (XIV):

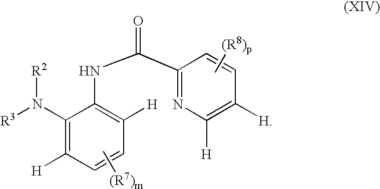

The values and preferred values for the variables in Structural Formula (XIV) are as described above for Structural Formula (I). In a first preferred embodiment, the values for the variables in Structural Formula (XIV) are described below.

m and p are independently 0, 1, or 2.

R² and R³ are independently C1-C6 alkyl optionally substituted with amino, hydroxyl, methoxy or ethoxy.

Each R⁷is independently selected from halogen, C1-C4 alkyl, C1-C4 haloalkyl, cyano, —OR³⁰, —SR³⁰, —N(R³¹)₂, Ar¹, —V—OR³⁰, —V—N(R⁴¹)₂, —V—Ar¹, —O—V—Ar¹, —O—V₁—N(R³¹)₂, —S—V—Ar¹, —S—V₁—N(R³¹)₂, —N(R³¹)—V—Ar¹ or —N(R³¹)—V₁—N(R³¹)₂.

Each R⁸is independently selected from halogen, C1-C4 alkyl, C1-C4 haloalkyl, cyano, —OR⁴⁰, —SR⁴⁰, —N(R⁴¹)₂, Ar², —V₂—OR⁴⁰, —V₂—N(R⁴¹)₂, —V₂—Ar², —O—V₃—Ar², —O—V₃—N(R⁴¹)₂, —S—V₂—Ar², —S—V₃—N(R⁴¹)₂, —N(R⁴¹)—V₂—Ar² or —N(R⁴¹)—V₃—N(R⁴¹)₂.

Ar¹ and Ar² are phenyl, each independently substituted with zero, one or two groups independently selected from halogen, alkyl, amino, alkylamino, dialkylamino, alkoxy, nitro, cyano, hydroxy, haloalkoxy or haloalkyl.

Each V and each V₂ is independently a C1-C4 alkylene group.

Each V₁and each V₃ is independently a C2-C4 alkylene group.

Each R³⁰ and each R⁴⁰ is independently: i) hydrogen; ii) a phenyl group substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R³¹ is independently R³⁰; or —N(R³¹)₂ taken together is a monocyclic non-aromatic heterocyclic group, wherein: i) the monocyclic non-aromatic heterocyclic group represented by —N(R³¹)₂ is optionally and independently substituted at any one or more substitutable ring carbon with C1-C2 alkyl, —OH, N-pyrrolidine, N-piperidinyl, N-(4-alkyl)piperazinyl, N-morpholinyl or N-pyrrolyl; and ii) the monocyclic non-aromatic heterocyclic group represented by —N(R³¹)₂ is optionally and independently substituted at any substitutable ring nitrogen atom with C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy.

Each R⁴¹ is independently R⁴⁰; or —N(R⁴¹)₂ taken together is a monocyclic non-aromatic heterocyclic group, wherein: i) the monocyclic non-aromatic heterocyclic group represented by —N(R⁴¹)₂ is optionally and independently substituted at any one or more substitutable ring carbon with C1-C2 alkyl, —OH, N-pyrrolidinyl, N-piperidinyl, N-(4-alkyl)piperazinyl, N-morpholinyl or N-pyrrolyl; and b) the monocyclic non-aromatic heterocyclic group represented by —N(R⁴¹)₂ is optionally and independently substituted at any substitutable ring nitrogen atom with C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy.

In a second more preferred embodiment, the variables in Structural Formula (XIV) are described below.

Each R³¹ is independently R³⁰; or —N(R³¹)₂ taken together is an optionally substituted N-piperidinyl, N-pyrrolidinyl, N-azepanyl, N-morpholinyl, N-thiomorphinyl, N-piperazinyl or N-diazepanyl group.

Each R⁴¹ is independently R⁴⁰; or —N(R⁴¹)₂ taken together is an optionally substituted N-piperidinyl, N-pyrrolidinyl, N-azepanyl, N-morpholinyl, N-thiomorphinyl, N-piperazinyl or N-diazepanyl.

The values for the remainder of the variables are as described above for the first more preferred embodiment for Structural Formula (XIV).

Another embodiment of the invention is an angiogenesis inhibitor represented by Structural Formula (II). The values and preferred values for the variables in Structural Formula (II) are provided below.

—Y— is —SO₂— or —C(O)—. Preferably, —Y— is —C(O)—.

R^{1a} is H or a C1-C6 alkyl group optionally substituted with amino, hydroxyl, methoxy or ethoxy. Preferably, R¹ is —H or C1-C3 alkyl.

X₁—X₃ are independently N, CH or CR^{6a}, provided that X₁ and X₂ are not both N. In one preferred embodiment, X₁—X₃ are CR^{6a}. In another preferred embodiment, X₁ is N and X₂—X₃ are CR^{6a}. In another preferred embodiment, X₂ is N and X₁ and X₃ are CR^{6a}. In another preferred embodiment, X₃ is N and X₁ and X₂ are CR^{6a}. In another preferred embodiment, X₁ and X₃ are N and X₂ is CR^{6a}. In another preferred embodiment, X₂ and X₃ are N and X₁ is CR^{6a}. Each R^{6a} group is independently selected. Suitable values for R^{6a} are provided below.

Ring A is optionally substituted at any one or more substitutable ring carbon atoms. Ring A is preferably substituted with m groups represented by R^{8a}. Suitable values for m and R^{8a} are provided below. Each substituent on Ring A is independently selected.

Each R^{6a} is independently halogen, alkyl, haloalkyl, —OR²⁰, —O(haloalkyl), —SR²⁰, —NO₂, —CN, —N(R²¹)₂, —NR²¹C(O)R²⁰, —NR²¹CO₂R²¹—N(R²¹)C(O)N(R²¹)₂, —C(O)R²⁰, —C(O)N(R²¹)₂, —S(O)₂R²⁰, —SO₂N(R²¹)₂, —S(O)R²², —NR²¹SO₂N(R²¹)₂, —NR²¹SO₂R²², —Vₒ—N(R²¹)₂, —O—Vₒₒ—N(R²¹)₂, —S—Vₒₒ—N(R²¹)₂ or —N(R²¹)—Vₒₒ—N(R²¹)₂. When the disclosed angiogenesis inhibitor is used in the methods of treatment described herein, —CO₂R²⁰ is an additional value for R^{6a}. Preferably, each R^{6a} is independently halogen, nitro, cyano, C1-C2 alkyl, C1-C2 haloalkyl, C1-C2 alkoxy, C1-C2 haloalkoxy, —O—(CH₂)₂—N(R²¹)₂, —CH₂—N(R²¹)₂ or —CH₂CH₂—N(R²¹)₂.

Each R^{8a} is independently halogen, alkyl, haloalkyl, Ar², —O(haloalkyl), —SR⁴⁰, —NO₂, —CN, —N(R⁴¹)₂, —NR⁴¹C(O)R⁴⁰, —NR⁴¹CO₂R⁴², —N(R⁴¹)C(O)N(R⁴¹)₂, —C(O)R⁴⁰, —CO₂R⁴⁰, —C(O)N(R⁴¹)₂, —S(O)₂R⁴⁰, —SO₂N(R⁴¹)₂, —S(O)R⁴², —NR⁴¹SO₂N(R⁴¹)₂, —NR⁴¹SO₂R⁴², —V₂—Ar², —V₂—OR⁴⁰, —V₂—O(haloalkyl), —V₂—SR⁴⁰, —V₂—NO₂, —V₂—CN, —V₂—N(R⁴¹)₂, —V₂—NR⁴⁰C(O)R⁴⁰, —V₂—NR⁴¹CO₂R⁴², —V₂—N(R⁴¹)₂, —V₂—C(O)R⁴⁰, —V₂—CO₂R⁴⁰, —V₂—C(O)N(R⁴¹)₂, —V₂—S(O)₂R⁴⁰, —V₂—SO₂N(R⁴¹)₂, —V₂—S(O)R⁴², —V₂—NR⁴¹SO₂N(R⁴¹)₂, —V₂—NR⁴¹SO₂R⁴², —O—V₂—Ar², —O—V₃—N(R⁴¹)₂, —S—V₂—Ar², —S—V₃—N(R⁴¹)₂, —N(R⁴¹)—V₂—Ar², —N(R⁴¹)—V₃—N(R⁴¹)₂, —NR⁴¹C(O)—V₂—N(R⁴¹)₂, —NR⁴ C(O)—V₂—Ar², —C(O)—V₂—N(R⁴¹)₂, —C(O)—V₂—Ar², —CO₂—V₂—N(R⁴¹)₂, —CO₂—V₂—Ar², —C(O)N(R⁴¹)—V₃—N(R⁴¹)₂, —C(O)N(R⁴¹)—V₂—Ar², —S(O)₂—V₂—N(R⁴¹)₂, —S(O)₂—V₂—Ar², —SO₂N(R⁴¹)—V₃—N(R⁴¹)₂, —SO₂N(R⁴¹)—V₂—Ar², —S(O)—V₂—N(R⁴¹)₂, —S(O)—V₂—Ar², —NR⁴¹SO₂—V₂—N(R⁴¹)₂ or —NR⁴¹SO₂—V₂—Ar²; or two adjacent R groups, taken together, form a methylenedioxy, ethylenedioxy or —[CH₂]₄— group. Preferably, each R⁸ is independently selected from halogen, C1-C4 alkyl, C1-C4 haloalkyl, cyano, —OR⁴⁰, —SR₄₀, —N(R⁴¹)₂, Ar², —V₂—OR⁴⁰, —V₂—N(R⁴¹)₂, —V₂—Ar², —O—V₂—Ar², —O—V₃—N(R⁴¹)₂, —S—V₂—Ar², —S—V₃—N(R⁴¹)₂, —-N(R⁴¹)—V²—Ar² or —N(R⁴¹)—V₃—N(R⁴¹)₂.

Each R²⁰ is independently hydrogen or an alkyl group.

Each R²¹ is independently hydrogen, an alkyl group, —CO₂R²⁰, —SO₂R²⁰ or —C(O)R²⁰; or —N(R²¹)₂ is an optionally substituted non-aromatic nitrogen-containing heterocyclic group. Preferably, each R²¹ is independently —H or an alkyl group or —N(R²¹)₂ taken together is a five or six-membered non-aromatic nitrogen-containing heterocyclic group.

Each R²² is independently an alkyl group.

Each R³⁰ is independently: i) hydrogen; ii) an aromatic group substituted with zero, one or two groups represented by halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy. Preferably, each R³⁰ is independently: i) hydrogen; ii) a phenyl group substituted with zero, one or two groups independently selected from halogen, alkyl, amino, alkylarnino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R⁴⁰ is independently: i) hydrogen; ii) an aromatic substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy. Preferably, each R⁴⁰ is independently: i) hydrogen; ii) a phenyl group substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R⁴¹ is independently R⁴⁰, —CO₂R⁴⁰, —SO₂R⁴⁰ or —C(O)R⁴⁰; or —N(R⁴¹)₂ taken together is an optionally substituted non-aromatic heterocyclic group. Preferably, each R⁴¹ is independently R⁴⁰, or —N(R⁴¹)₂ is an optionally substituted non-aromatic heterocyclic group. Preferably, each R⁴¹ is independently R⁴⁰, or —N(R⁴¹)₂ is an optionally substituted non-aromatic heterocyclic group. More preferably, each R⁴¹ is independently R⁴⁰; or —N(R⁴¹)₂ taken together is an optionally substituted N-piperidinyl, N-pyrrolidinyl, N-azepanyl, N-morpholinyl, N-thiomorphinyl, N-piperazinyl or N-diazepanyl. Suitable substitutents for a non-aromatic heterocyclic ring represented by —N(R⁴¹)₂ are described below in the section providing suitable substitutents for a non-aromatic heterocyclic group. Preferred substitutents for a substitutable ring carbon of a monocyclic non-aromatic heterocyclic group represented by —N(R⁴¹)₂ are C1-C2 alkyl, —OH, N-pyrrolidinyl, N-piperidinyl, N-(4-alkyl)piperazinyl, N-morpholinyl or N-pyrrolyl; and preferred substituents for a substitutable ring nitrogen atom of a monocyclic non-aromatic heterocyclic group represented by —N(R⁴¹)₂ are C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy. Each ring carbon and ring nitrogen substituents is independently selected.

Each R⁴² is independently: i) an aromatic substituted with zero, one or two groups independently selected from halogen, alkyl, amino, alkylamino, dialkylamino, alkoxy, nitro, cyano, hydroxy, haloalkoxy or haloalkyl; or ii) an alkyl group optionally substituted with halogen, hydroxyl, alkoxy, nitro, cyano, alkoxycarbonyl, alkylcarbonyl or haloalkoxy.

R⁵¹ and R⁵² taken together with their intervening atoms form an optionally substituted monocyclic non-aromatic heterocyclic group and R⁵³ is —H or C1-C6 alkyl group optionally substituted with amine, C1-C2 alkylamine, C1-C2 dialkylamine, hydroxyl, methoxy, ethoxy, cycloalkyl, or optionally substituted aryl; or R⁵¹ is C1-C6 alkyl group optionally substituted with amino, hydroxyl, methoxy, ethoxy or an optionally substituted aryl group (in some instances —H is an additional value for R⁵¹); and R⁵² and R⁵³ are independently —H or C1-C6 alkyl group optionally substituted with amino, hydroxyl, methoxy, ethoxy, cycloalkyl, or an optionally substituted aryl group; or —NR⁵²R⁵³ is an optionally substituted monocyclic non-aromatic heterocyclic group. Preferably, R⁵¹ and R⁵², taken together with their intervening atoms, form an optionally substituted monocyclic non-aromatic heterocyclic group and R⁵³ is —H or C1-C6 alkyl group optionally substituted with amine, C1-C2 alkylamine, C1-C2 dialkylamine, hydroxyl, methoxy, ethoxy, cycloalkyl, or an optionally substituted aryl group; or R⁵¹ is C1-C6 alkyl group optionally substituted with amino, hydroxyl, methoxy, ethoxy or an optionally substituted aryl group and —NR⁵²R⁵³ is an optionally substituted mono-cyclic non-aromatic heterocyclic group; or R⁵¹ is an optionally substituted C1-C3 aralkyl and R⁵² and R⁵³ are independently —H or an optionally substituted C1-C6 alkyl group.

More preferably, R⁵¹ and R⁵², taken together with their intervening atoms, form an optionally substituted piperidinyl, pyrrolidinyl, azepanyl, morpholinyl, thiomorphinyl, piperazinyl or diazepanyl group; and R⁵³ is —H or C1-C6 alkyl group optionally substituted with amine, C1-C2 alkylamine, C1-C2 dialkylamine, hydroxyl, methoxy, ethoxy, cycloalkyl, or an optionally substituted aryl group. Even more preferably, R⁵¹ and R⁵², taken together with their intervening atoms, form a 2-pyrrolidinyl group and R⁵³ is C1-C3 alkyl, C1-C3 cyclqalkylalkyl or phenyl-(C1-C3)alkyl optionally substituted with halogen, nitro, cyano, C1-C2 alkyl, C1-C2 haloalkyl, C1-C2 alkoxy or C1-C2 haloalkoxy.

Alternatively, R⁵¹ is C1-C6 alkyl group optionally substituted with amino, hydroxyl, methoxy, ethoxy or an optionally substituted aryl group and —NR⁵²R⁵³ taken together forms a piperidinyl, pyrrolidinyl, azepanyl, morpholinyl, thiomorphinyl, piperazinyl or diazepanyl group optionally substituted at any substitutable ring carbon atom and optionally substituted at any substitutable ring nitrogen atom. Even more preferably, R⁵¹ is a C1-C3 alkyl group or a C1-C3 phenalkyl group optionally substituted with halogen, nitro, cyano, C1-C2 alkyl, C1-C2 haloalkyl, C1-C2 alkoxy or C1-C2 haloalkoxy; and —NR⁵²R⁵³ taken together is N-pyrrolidinyl optionally substituted at any substitutable carbon atom with methyl. Even more preferably, R⁵¹ is an optionally substituted aralkyl and R⁵² and R⁵³ are H or an optionally substituted C1-C6 alkyl group.

Ar¹ and Ar² are independently a monocyclic aromatic group (preferably a phenyl group) each substituted with zero, one or two groups independently selected from halogen, alkyl, amino, alkylamino, dialkylamino, alkoxy, nitro, cyano, hydroxy, haloalkoxy or haloalkyl.

Each V is independently a C1-C4 alkylene group.

Each V₁ is independently a C2-C4 alkylene group.

Each V₂ is independently a C1-C4 alkylene group.

Each V₃ is independently a C2-C4 alkylene group.

m is 0, 1, 2, 3 or 4; preferably 0, 1 or 2.

In a preferred embodiment, the angiogenesis inhibitor of the invention is represented by Structural Formula (XV), (XVI) or (XVII):

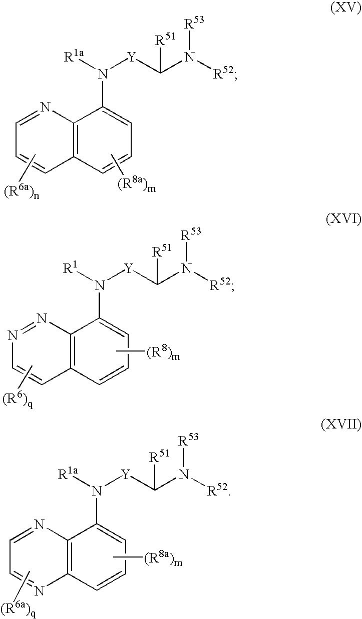

n in Structural Formula (XV) is 0, 1, 2 or 3; q in Structural Formulas (XVI) and (XVII) is 0, 1 or 2; and the values and preferred values for Structural Formulas (XV), (XVI) and (XVII) are as described for Structural Formula (II). In a first more preferred embodiment, the variables in Structural Formulas (XV), (XVI) and (XVII) are as described below:

n in Structural Formula (XV) is 0, 1, 2 or 3; q in Structural Formulas (XVI) and (XVII) is 0, 1 or 2.

Each R^{8a} is independently selected from halogen, C1-C4 alkyl, C1-C4 haloalkyl, cyano, —SR⁴⁰, —N(R⁴¹)₂, Ar², —V₂—OR⁴⁰, —V₂—N(R⁴¹)₂, —V₂—Ar², —O—V₂—Ar², —O—V₃—N(R⁴¹)₂, —S—V₂—Ar², —S—V₃—N(R⁴¹)₂, —N(R⁴¹)—V₂—Ar² or —N(R⁴¹)—V₃—N(R⁴¹)₂;

Ar² is phenyl independently substituted with zero, one or two groups independently selected from halogen, alkyl, amino, alkylamino, dialkylamino, alkoxy, nitro, cyano, hydroxy, haloalkoxy or haloalkyl.

Each R⁴⁰ is independently: i) hydrogen; ii) a phenyl group substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R⁴¹ is independently R⁴⁰; or —N(R⁴¹)₂ taken together is an optionally substituted non-aromatic heterocyclic group.

R⁵¹ and R⁵², taken together with their intervening atoms, form an optionally substituted non-aromatic heterocyclic group and R⁵³ is —H or C1-C6 alkyl group optionally substituted with amine, C1-C2 alkylamine, C1-C2 dialkylamine, hydroxyl, methoxy, ethoxy, cycloalkyl or optionally substituted aryl group. Alternatively, R⁵¹ is a C1-C6 alkyl group optionally substituted with amino, hydroxyl, methoxy, ethoxy or an optionally substituted aryl group and —NR⁵²R⁵³ taken together is a non-aromatic heterocyclic group optionally substituted at any substitutable ring carbon atom.

The values and preferred values for the remainder of the variables are as described for Structural Formula (II).

In a second more preferred embodiment, the variables for Structural Formulas (XV), (XVI) or (XVII) are as described below.

Each R^{6a} is independently halogen, nitro, cyano, C1-C2 alkyl, C1-C2 haloalkyl, C1-C2 alkoxy, C1-C2 haloalkoxy, —O—(CH₂)₂—N(R²¹)₂, —CH₂—N(R²¹)₂ or —CH₂CH₂—N(R²¹)₂.

Each R²¹ is independently -H or an alkyl group or —N(R²¹)₂ taken together is a five or six-membered non-aromatic nitrogen-containing heterocyclic group.

The values and preferred values for the remainder of the variables as described in the first more preferred embodiment for Structural Formulas (XV), (XVI) and (XVII). More preferably, R⁵¹ and R⁵², taken together with their intervening atoms, form an optionally substituted piperidinyl, pyrrolidinyl, azepanyl, morpholinyl, thiomorphinyl, piperazinyl or diazepanyl group and R⁵³ is —H or C1-C6 alkyl group optionally substituted with amine, C1-C2 alkylamine, C1-C2 dialkylamine, hydroxyl, methoxy, ethoxy, cycloalkyl or optionally substituted aryl group. Alternatively, R⁵¹ is a C1-C6 alkyl group optionally substituted with amino, hydroxyl, methoxy, ethoxy or an optionally substituted aryl group and —NR⁵²R⁵³ taken together forms a piperidinyl, pyrrolidinyl, azepanyl, morpholinyl, thiomorphinyl, piperazinyl or diazepanyl group optionally substituted at any substitutable ring carbon atom and optionally substituted at any substitutable ring nitrogen atom.

In another preferred embodiment, the angiogenesis inhibitor is represented by Structural Formula (XVIII), (XIX) or (XX): 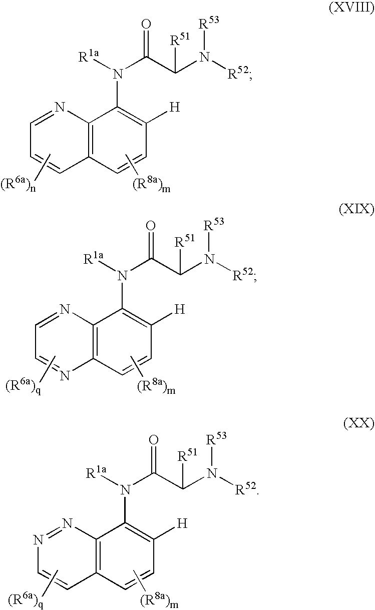
The values and preferred values for the variables in Structural Formulas (XVIII), (XIX) and (XX) are as described for Structural Formulas (XV), (XVI) and (XVII). In a first more preferred embodiment, the variables in Structural Formulas (XVIII), (XIX) and (XX) are as defined below.

n, m and q are independently 0, 1 or 2.

R^{1a} is H or C1-C6 alkyl optionally substituted with amino, hydroxyl, methoxy or ethoxy.

Each R^{6a} is independently halogen, nitro, cyano, C1-C2 alkyl, C1-C2 haloalkyl, C1-C2 alkoxy, C1-C2 haloalkoxy, —O—(CH₂)₂—N(R²¹)₂, —CH₂—N(R²¹)₂ or —CH₂CH₂—N(R₂₁)₂.

Each R^{8a} is independently selected from halogen, C1-C4 alkyl, C1-C4 haloalkyl, cyano, —SR₄, —N(R⁴¹)₂, Ar₂, —V₂—OR⁴⁰, —V₂—N(R⁴¹)₂, —V₂—Ar², —O—V₂—Ar², —O—V₃—N(R⁴¹)₂, —S—V₂—Ar², —S—V₃—N(R⁴¹)₂, —N(R⁴¹)—V₂—Ar² or —N(R⁴¹)—V₃—N(R⁴¹)₂.

Each R²¹ is independently —H or an alkyl group or —N(R²¹)₂ taken together is a five or six-membered non-aromatic nitrogen-containing heterocyclic group.

Each R⁴⁰ is independently: i) hydrogen; ii) a phenyl group substituted with zero, one or two groups independently selected from halogen, alkyl, amino, alkylamino, dialkylamino, alkoxy, nitro, cyano, hydroxy, haloalkoxy or haloalkyl; or iii) an alkyl group optionally substituted with halogen, hydroxyl, alkoxy, nitro, cyano, alkoxycarbonyl, alkylcarbonyl or haloalkoxy.

Each R⁴¹ is independently R⁴⁰; or —N(R⁴¹)₂ taken together is a monocyclic non-aromatic heterocyclic group, wherein: i) the moncyclic non-aromatic heterocyclic group represented by —N(R⁴¹)₂ is optionally and independently substituted at any one or more substitutable ring carbon with C1-C2 alkyl, —OH, N-pyrrolidinyl, N-piperidinyl, N-(4-alkyl)piperazinyl, N-morpholinyl or N-pyrrolyl; and ii) the moncyclic non-aromatic heterocyclic group represented by —N(R⁴¹)₂ is optionally and independently substituted at any substitutable ring nitrogen atom with C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy.

Each V₂ is independently a C1-C4 alkylene group.

Each V₃ is independently a C2-C4 alkylene group.

Ar² is phenyl independently substituted with zero, one or two groups independently selected from halogen, alkyl, amino, alkylamino, dialkylamino, alkoxy, nitro, cyano, hydroxy, haloalkoxy or haloalkyl.

R⁵¹ and R⁵², taken together with their intervening atoms, form an optionally substituted piperidinyl, pyrrolidinyl, azepanyl, morpholinyl, thiomorphinyl, piperazinyl or diazepanyl group; and R⁵³ is —H or C1-C6 alkyl group optionally substituted with amine, C1-C2 alkylamine, C1-C2 dialkylamine, hydroxyl, methoxy, ethoxy, cycloalkyl or optionally substituted aryl group. Alternatively, R⁵¹ is a C1-C6 alkyl group optionally substituted with amino, hydroxyl, methoxy, ethoxy or an optionally substituted aryl group and —NR⁵²R⁵³ taken together forms a piperidinyl, pyrrolidinyl, azepanyl, morpholinyl, thiomorphinyl, piperazinyl or diazepanyl group optionally substituted at any substitutable ring carbon atom and optionally substituted at any substitutable ring nitrogen atom.

In a second more preferred embodiment, the values for the variables in Structural Formulas (XVIII), (XIX) or (XX) are as described below.

R⁵¹ and R⁵², taken together with their intervening atoms, form a 2-pyrrolidinyl group and R⁵³ is C1-C3 alkyl, C1-C3 cycloalkylalkyl or phenyl-(C1-C3)alkyl optionally substituted with halogen, nitro, cyano, C1-C2 alkyl, C1-C2 haloalkyl, C1-C2 alkoxy or C1-C2 haloalkoxy; or R⁵¹ is a C1-C3 alkyl group or a C1-C3 phenalkyl group optionally substituted with halogen, nitro, cyano, C1-C2 alkyl, C1-C2 haloalkyl, C1-C2 alkoxy or C1-C2 haloalkoxy and —NR⁵²R⁵³ taken together is N-pyrrolidinyl optionally substituted at any substitutable carbon atom with methyl (more preferably; and the values and preferred values for the variables are as described in the first more preferred embodiment for Structural Formulas (XVIII), (XIX) and (XX). More preferably each R⁴¹ is independently R⁴⁰; or —N(R⁴¹)₂ taken together is an optionally substituted N-piperidinyl, N-pyrrolidinyl, N-azepanyl, N-morpholinyl, N-thiomorphinyl, N-piperazinyl or N-diazepanyl group.

The invention is illustrated by the following examples which are not intended to be limiting in any way.

Exemplification

### EXAMPLES OF SYNTHETIC METHODS

Representative methods for the synthesis of Example Compounds and requisite intermediates are shown below. Intermediates whose synthesis is not described were commercially available or prepared by literature methods. ¹H and ³C NMR spectra were generated at 400 and 100 MHz, respectively (Varian Unity 400 instrument). Chemical shifts are given in parts per million relative to a tetramethylsilane internal standard.

### Preparation of Intermediates

### Synthetic Method 1 6-Chloro-8-nitro-quinoline (precursor to Example Compound 4)

To a stirred solution of concentrated sulfuric acid (24 mL) and water (6 mL) was added 4-chloro-2-nitro-aniline (10.0 g, 58.0 mmol), 3-nitrobenzenesulfonic acid sodium salt (14.3 g, 63.7 mmol) and glycerol (11.0 mL, 151 mmol). The mixture was heated at 150° C. for 1 hour. After allowing to cool, the resulting viscous black oil was slowly poured into a stirred and cooled (0° C.) solution of sodium hydroxide (36 g, 900 mmol) in water (300 mL). Ethyl acetate (˜200 mL) and Celite (100 mL) were added. The mixture was suction filtered and the filtercake rinsed with ethyl acetate. The combined filtrate was washed with aqueous sodium bicarbonate solution, dried (sodium sulfate) and concentrated to afford a brown solid. Flash chromatography over silica (dichloromethane) followed by trituration with diisopropyl ether afforded 6.23 g (52%) of product as fluffy yellow solid: ¹H NMR (CDCl₃) δ 9.07 (dd, J=4.3, 1.6 Hz, 1H), 8.21 dd, J=8.4, 1.6 Hz, 1H), 8.04 (d, J=2.2 Hz, 1H), 8.02 (d, J=2.2 Hz, 1H), 7.60 (dd, J=8.4, 4.3 Hz, 1H) ppm.

### Synthetic Method 2 6-chloro-quinolin-8-ylamine (precursor to Example Compound 4)

To a stirred suspension of 6-chloro-8-nitro-quinoline (synthetic method 1; 1.00 g, 4.79 mmol) in water (20 mL) was added acetic acid (0.82 mL, 14.3 mmol) and iron dust (1.61 g, 28.8 mmol). The mixture was heated to near reflux for 1.5 hours and then cooled to room temperature. With continued stirring, the reaction was treated with solid sodium hydroxide (0.65 g, 16.3 rnmol) and ethyl acetate (˜30 mL). Celite (˜30 mL) was added and the slurry was suction filtered. The filtercake was rinsed with ethyl acetate. The combined filtrate was washed with aqueous sodium bicarbonate solution, dried (sodium sulfate) and concentrated. The resulting viscous brown oil was purified by flash chromatography over silica (hexanes/ethyl acetate) to afford 0.57 g (67%) of product as a yellow solid: ¹H NMR (CDCl₃) δ 8.71 (dd, J=4.2, 1.5 Hz, 1H), 7.94 (dd, J=8.3, 1.5 Hz, 1H), 7.36 (dd, J=8.3, 4.2 Hz, 1H), 7.09 (d, J=2.1 Hz, 1H), 6.84 (d, J=2.1 Hz, 1H), 5.09 (br s, 2H) ppm.

### Synthetic Method 3 6-trifluoromethyl-quinolin-8-ylamine (precursor to Example Compound 5)

To a stirred solution of 8-nitro-6-trifluoromethyl-quinoline (prepared according to synthetic method 1; 0.75 g, 3.10 mmol) in 5 N aqueous hydrochloric acid (15 mL) was added tin(II) chloride dihydrate (2.10 g, 9.31 mmol). After 1 hour, the reaction was diluted with a solution of sodium hydroxide (4.0 g, 100 mmol) in water (20 mL) and ethyl acetate. The organic layer was removed, combined with a second ethyl acetate extract and dried (sodium sulfate). Concentration afforded a yellow solid which was purified by flash chromatography over silica (hexanes/ethyl acetate) to afford 0.237 g (36%) of product as an orange-yellow solid: ¹H NMR (CDCl₃) δ 8.82 (dd, J=4.1, 1.5 Hz, 1H), 8.10 (dd, J=8.3, 1.5 Hz, 1H), 7.46-7.38 (m, 2H), 7.01 (d, J=1.6 Hz, 1H), 5.20 (br s, 2H) ppm.

### Synthetic Method 4 5-morpholin-4-yl-8-nitro-quinoline (precursor to Example Compounds 9, 52, 53)

A stirred solution of 5-chloro-8-nitro-quinoline (6.00 g, 28.8 mmol) and morpholine (7.5 mL, 86 mmol) in dimethylsulfoxide (30 mL) was heated at 110° C. for ˜4 hours. The reaction solution was cooled and then partitioned between ethyl acetate and water. The organic layer was washed with water, dried (sodium sulfate) and concentrated to afford 7.20 g (96%) of crude product, which was used without further purification, as an orange-brown solid: ¹H NMR (CDCl₃) δ 9.07-9.04 (m, 1H), 8.53-8.48 (m, 1H), 8.09 (d, J=8.3 Hz, 1H), 7.52 (dd, J=8.7, 4.2 Hz, 1H), 7.07 (d, J=8.3 Hz, 1H), 4.03-3.95 (m, 4H), 3.22-3.13 (m, 4H) ppm.

### Synthetic Method 5 8-nitro-6-phenyl-quinoline (precursor to Example Compound 10)

Into a flame-dried flask under inert atmosphere was loaded, palladium(II) acetate (67.0 mg, 30.0 μmol), 2-(di-t-butylphosphino)biphenyl (18.0 mg, 60.0 μmol), phenylboronic acid (0.549 g, 4.50 mmol), potassium fluoride (0.523 g, 9.00 mmol), 6-chloro-8-nitro-quinoline (synthetic method 1; 0.626 g, 3.00 mmol) and tetrahydrofuran (3.0 mL). The mixture was heated (55° C.) with stirring overnight, cooled to room temperature and partitioned between ethyl acetate and 1 N aqueous sodium hydroxide solution. The organic layer was combined with an ethyl acetate back extract of the aqueous layer, dried (sodium sulfate) and concentrated. The resulting gum was partially purified (to ˜85%) by flash chromatography over silica (hexanes/ethyl acetate) to afford 0.499 g (66%) of product, which was used without further purification, as a yellow solid: ¹H NMR (CDCl₃) δ 9.05 (dd, J=4.2, 1.6 Hz, 1H), 8.33-8.28 (m, 2H), 8.19 (d, J=1.9 Hz, 1H), 7.72-7.66 (m, 2H), 7.61-7.42 (m, 4H) ppm.

### Synthetic Method 6 8-nitro-5-phenoxy-quinoline (precursor to Example Compound 12)

To a stirred solution of phenol (0.244 g, 2.59 mmol) and 5-chloro-8-nitro-quinoline (0.450 g, 2.16 mmol) in N,N-dimethylformamide (5.0 mL) was added sodium hydride (0.120 g of 60% dispersion in mineral oil, 3.00 mmol). After 5 hours, the reaction was partitioned between ethyl acetate and aqueous sodium bicarbonate solution. The organic layer was dried (sodium sulfate) and concentrated to afford a moist, yellow-brown solid. The crude product was triturated with hexanes/ethyl acetate to afford 0.438 g (76%) of product as a mustard colored solid: ¹H NMR (CDCl₃) δ 9.15 (dd, J=4.2, 1.6 Hz, 1H), 8.79 (dd, J=8.5, 1.6 Hz, 1H), 8.06 (d, J=8.6 Hz, 1H), 7.63-7.57 (m, 1H), 7.52-7.44 (m, 2H), 7.35-7.27 (m, 1H), 7.17 (d, J=8.0 Hz, 2H), 6.75 (d, J=8.6 Hz, 1H) ppm.

### Synthetic Method 7 4-morpholin-4-yl-2-nitro-phenylamine (precursor to Example Compound 13)

### Step 1) Preparation of N-(4-Morpholin-4-yl-phenyl)-acetamide

To a stirred suspension of 4-morpholinoaniline (5.16 g, 28.9 mmol) in ethyl acetate (75 mL) was added acetic anhydride (3.2 mL, 34 mmol). The mixture was heated at reflux for 45 minutes and then cooled to room temperature. The resulting precipitate was suction filtered off, rinsed with ethyl acetate and air dried under house vacuum to afford 5.52 g (87%) of crude product as a purple-gray solid. This material was used without further purification.

### Step 2) Preparation of N-(4-Morpholin-4-yl-2-nitro-phenyl)-acetamide

To a stirred and cooled (0° C.) solution of the product of step 1 (5.42 g, 24.6 mmol) in concentrated sulfuric acid (20 mL) was added, dropwise over 10 minutes, a solution of fuming nitric acid (1.1 mL, 26 mmol) in concentrated sulfuric acid (1 mL). After 15 minutes, the reaction was poured over crushed ice. The resulting slurry was basified with concentrated ammonia hydroxide (˜75 mL) and extracted with ethyl acetate. The organic layer was dried (sodium sulfate) and concentrated to afford 5.90 g (90%) of crude product, which was used without further purification, as a red-brown solid.

### Step 3) Preparation of 4-Morpholin-4-yl-2-nitro-phenylamine

The product of step 2 was taken up in 4 N hydrochloric acid (30 mL) and heated at 100° C. for 30 minutes. The reaction was diluted with water, basified with concentrated ammonium hydroxide and extracted with ethyl acetate. The combined organic layers were dried (sodium sulfate) and concentrated. The resulting brown solid was purified by flash chromatography over silica (dichloromethane/methanol) to afford 3.59 g (72%) of product as a red-brown solid: ¹H NMR (CDCl₃) δ 7.46 (d, J=2.7 Hz, 1H), 7.10 (dd, J=9.0, 2.7 Hz, 1H), 6.76 (d, J=9.0 Hz, 1H), 3.84-3.75 (m, 4H), 3.22 (br s, 2H), 3.03-2.94 (m, 4H) ppm.

### Synthetic Method 8 5-(4-methyl-piperazin-1-yl)-quinolin-8-ylamine (precursor to Example Compound 19)

To a stirred solution of 5-(4-methyl-piperazin-1-yl)-8-nitro-quinoline (prepared according to synthetic method 4; 0.57 g, 2.09 mmol) in ethanol (15 mL) was added 10% palladium on activated carbon (0.100 g). The reaction vessel was evacuated and backfilled with nitrogen several times. After a final evacuation, the reaction vessel was backfilled with hydrogen. Following overnight stirring the mixture was filtered through Celite and concentrated to afford 0.49 g (96%) of crude product, which was used without further purification, as a dark brown gum: ¹H NMR (CDCl₃) δ 8.77 (dd, J=4.1, 1.6 Hz, 1H), 8.51 (dd, J=8.5, 1.6 Hz, 1H), 7.39 (dd, J=8.5, 4.1 Hz, 1H), 7.05 (d, J=8.0 Hz, 1H), 6.87 (d, J=8.0 Hz, 1H), 4.80 (br s, 2H), 3.07-2.98 (m, 4H), 2.81-2.51 (m, 4H), 2.41 (s, 3H) ppm.

### Synthetic Method 9 4-(2-morpholin-4-yl-ethoxy)-2-nitro-phenylamine (precursor to Example Compound 20)

To a stirred solution of 4-amino-3-nitro-phenol (10.0 g, 64.9 mmol), 4-(2-hydroxyethyl)morpholine (9.36 g, 71.4 mmol) and triphenylphosphine (18.7 g, 71.2 mmol) in ethyl acetate (250 mL) was added, dropwise over 5 minutes, diethyl azodicarboxylate (11.2 mL, 71.1 mmol). The reaction was allowed to proceed overnight and then extracted with successive portions of 1 N hydrochloric acid. The combined extracts were basified with concentrated ammonium hydroxide and extracted several times with ethyl acetate. The combined organic layers were dried (sodium sulfate) and concentrated to afford 15.9 g (92%; calculated to account for contamination with 20 mole % diethyl hydrazinedicarboxylate byproduct) of crude product, which was used without further purification, as an orange-brown gum: ¹H NMR (CDCl₃) δ 9.57 (d, J=2.9 Hz, 1H), 7.09 (dd, J=9.0, 2.9 Hz, 1H), 6.76 (d, J=9.0 Hz, 1H), 5.92 (br s, 2H), 4.08 (t, J=5.6 Hz, 2H), 3.77-3.72 (m, 4H), 2.79 (t, J=5.6, 2H), 2.61-2.54 (m, 4H) ppm.

### Synthetic Method 10 6-(2-morpholin-4-yl-ethoxy)-8-nitro-quinoline (precursor to Example Compound 20)

To a refluxing solution of 4-(2-morpholin-4-yl-ethoxy)-2-nitro-phenylamine (synthetic method 9; 15.9 g, 59.6 mmol) and chloranil (15.3 g, 62.2 mmol) in 1:1 ethanol/concentrated hydrochloric acid (50 mL) was added, dropwise over 25-30 minutes, a solution of acrolein (90%; 6.1 mL, 82 mmol) in ethanol (7.4 mL). After 30 minutes of continued heating, the reaction mixture was cooled and diluted with ˜100 mL water. The resulting suspension was basified with the addition of concentrated ammonium hydroxide (20 mL) and then further diluted with the addition of a 4:1 solution of aqueous sodium bicarbonate/aqueous sodium carbonate (100 mL) and ethyl acetate (200 mL). The biphasic mixture was filtered to remove a gray-green precipitate. The organic layer of the filtrate was removed, combined with an additional ethyl acetate extract and, in turn, extracted with 1 N hydrochloric acid. The aqueous layer was then basified with ammonium hydroxide and extracted with successive portions of ethyl acetate. The combined organic layers were dried (sodium sulfate) and concentrated to afford 17.6 g (97%) of crude product, which was used without further purification, as an orange-brown gum: ¹H NMR (CDCl₃) δ 8.88 (dd, J=4.2, 1.5 Hz, 1H), 8.12 (dd, J=8.4, 1.5 Hz, 1H), 7.72 (d, J=2.7 Hz, 1H), 7.48 (dd, J =8.4, 4.2 Hz, 1H), 7.29 (d, J=2.7 Hz, 1H), 4.27 (t, J=5.6 Hz, 2H), 3.78-3.71 (m, 4H), 2.89 (t, J=5.6 Hz, 2H), 2.65-2.56 (m, 4H) ppm.

### Synthetic Method 11 2-(N,N-dimethylamino)-aniline (precursor to Example Compound 80)

### Step 1) Preparation of Dimethyl-(2-nitrophenyl)-amine

To a stirred solution of l-fluoro-2-nitrobenzene (0.423 g, 3.00 mmol) in tetrahydrofuran (1 ml) was added a 2.0 M solution of dimethylamine in tetrahydrofuran (5 ml, 10 mmol). After 4 hours the reaction was concentrated in-vacuo. The residue was dissolved in dichloromethane (20 ml) and washed with aqueous sodium bicarbonate solution and brine. The organic phase was dried (sodium sulfate) and concentrated to provide 0.415 g (83%) of crude product, which was used without further purification, as a yellow oil: ¹H NMR (CDCl₃) δ 7.76 (d, 1H), 7.40 (t, 1H)) 7.03 (d, 1H), 6.82 (t, 1H), 2.89 (s, 6H) ppm.

### Step 2) Preparation of 2-(N,N-Dimethylamino)-aniline

To a stirred solution of the product of step 1 (0.415 g, 2.50 mmol) in 2:1 methanol/acetic acid (10 ml) was added iron filings (0.420 mg, 7.52 mmol). The mixture was heated to reflux. After 2 hours the reaction mixture was filtered thru Celite. The filter cake was washed with ethyl acetate and the filtrate was then extracted with ethyl acetate. The combined organic layers were washed with aqueous sodium bicarbonate solution and brine, dried (sodium sulfate) and concentrated. The resulting residue was purified by flash chromatography over silica (hexanes/ethyl acetate) to afford 0.208 g (61%) of product as an amber oil: ¹H NMR (CDCl₃) δ 7.07 (d, 1H), 6.96 (t, 1H), 6.79 (m, 2H), 4.09 (br s, 2H), 2.73 (s, 6H) ppm.

### PREPARATION OF EXAMPLE COMPOUNDS

### Synthetic Method 12 4-chloropyridine-2-carboxylic acid quinolin-8-ylamide (Example Compound 22)

To a stirred solution of 8-aminoquinoline (1.74 g, 12.1 mmol) and 4-chloropicolinic acid (2.00 g, 12.7 mmol) in chloroform (100 mL) was added 1-[3-(dimethylamino)propyl]-3-ethyl carbodiimide hydrochloride (2.54 g, 13.3 mmol) and catalytic 4-dimethylaminopyridine (ca. 50 mg). The reaction mixture was stirred at room temperature. After 16 h, the mixture was dilute with dichloromethane (200 mL) and washed with aqueous sodium bicarbonate solution and brine. The organic phase was dried (magnesium sulfate) and concentrated to provide 3.66 g of an orange solid. Flash chromatography over silica (dichloromethane/methanol) afforded 2.02 g (59%) of product as a light yellow solid: ¹H NMR (CDCl₃) δ 12.20 (br s, 1H), 8.99-8.95 (m, 2H), 8.68 (d, J=5.2 Hz, 1H), 8.35 (d, J=1.7 Hz, 1H), 8.19 (dd, J=8.3, 1.7 Hz, 1H), 7.63-7.57 (m, 2H), 7.51-7.47 (m, 2H) ppm. ¹³C NMR (CDCl₃) δ 161.4, 152.0, 149.4, 148.8, 146.0, 139.3, 136.3, 134.2, 128.1, 127.3, 126.4, 123.1, 122.3, 121.7, 117.0 ppm.

### Synthetic Method 13 pyridine-2-carboxylic acid (5-bromo-6-methyl-quinolin-8-yl)-amide (Example Compound 16)

To a stirred suspension of compound 11 (prepared according to synthetic method 12; 0.500 g, 1.90 mmol) and N-bromosuccinimide (0.389 g, 2.19 mmol) in carbon tetrachloride (10 mL) was added benzoyl peroxide (0.023 g, 0.095 mmol). The mixture was heated at reflux for 40 minutes, cooled to room temperature and concentrated. Flash chromatography over silica (dichloromethane/methanol) followed by trituration with diethyl ether afforded 0.501 g (77%) of product as a beige solid: ¹H NMR (CDCl₃) δ 12.21 (br s, 1H), 8.97 (s, 1H), 8.91 (d, J=3.5 Hz, 1H), 8.79 (d, J=3.5 Hz, 1H), 8.60 (d, J=8.0 Hz, 1H), 8.34 (d, J=8.0 Hz, 1H), 7.94 (t, J=7.4 Hz, 1H), 7.64-7.46 (m, 2H), 2.68 (s, 3H) ppm.

### Synthetic Method 14 4-(4-methylpiperazin-1-yl)pyridine-2-carboxylic acid quinolin-8-ylamide (Example Compound 34)

To a stirred solution of 1-methylpiperazine (0.360 g, 3.60 mmol) in dimethyl sulfoxide (5.0 mL) was added example compound 22 (synthetic method 12; 0.510 g, 1.80 mmol). The mixture was heated to 85° C. After 65 hours, the reaction mixture was allowed to cool to room temperature and water (50 mL) was added. The suspension was extracted with ethyl acetate. The combined organic phases were washed with brine, dried (magnesium sulfate), and concentrated to provide crude product as a yellow oil. Flash chromatography over silica (dichloromethane/methanol) afforded 0.345 g (55%) of product as a white solid: ¹H NMR (CDCl₃) δ 12.30 (br s, 1H), 9.00-8.96 (m, 2H), 8.43 (d, J=5.8 Hz, 1H), 8.17 (dd, J=8.2, 1.7 Hz, 1H), 7.78 (d, J=2.7 Hz, 1H), 7.62-7.54 (m, 2H), 7.47 (dd, J=8.2, 4.3 Hz, 1H), 6.79 (dd, J=5.8, 2.7 Hz, 1H), 3.48 (t, J=5.1 Hz, 4H), 2.55 (t, J=5.1 Hz, 4H), 2.36 (s, 3H) ppm. ¹³C NMR (CDCl₃) δ 163.5, 155.9, 151.2, 149.2, 148.7, 139.4, 136.1, 134.7, 128.1, 127.3, 121.8, 121.5, 116.7, 109.8, 106.9, 54.5, 46.1, 46.0 ppm.

### Synthetic Method 15 4-phenylsulfanylpyridine-2-carboxylic acid quinolin-8-ylamide (Example Compound 31)

To a stirred solution of thiophenol (0.078 g, 0.711 mmol) in 1-methyl-2-pyrrolidinone (5.0 mL) was added sodium hydride (0.030 g of 60% dispersion, 0.744 mmol). The reaction mixture was stirred at room temperature. After 5 minutes, example compound 22 (synthetic method 12; 0.192 g, 0.677 mmol) was added in one portion and the resulting orange solution was heated to 85° C. After 16 hours, the brown mixture was cooled to room temperature and water (100 mL) was added. The suspension was extracted with ethyl acetate. The combined organic phases were washed successively with water and brine, dried (magnesium sulfate) and concentrated to provide crude product as a tan solid. Flash chromatography over silica (dichloromethane/methanol) afforded 0.185 g (76%) of product as an off-white solid: ¹H NMR (CDCl₃) δ 12.20 (br s, 1H), 8.96-8.94 (m, 2H), 8.50 (d, J=5.2 Hz, 1H), 8.17 (dd, J=8.3, 1.7 Hz, 1H), 8.02 (d, J=1.7 Hz, 1H), 7.62-7.54 (m, 4H), 7.51-7.46 (m, 4H), 7.10 (dd, J=5.2, 2.0 Hz, 1H) ppm. ¹³C NMR (CDCl₃) δ 162.3, 152.8, 150.3, 148.7, 148.2, 139.3., 136.2, 135.4, 134.4, 130.1, 130.0, 128.8, 128.1, 127.3, 122.7, 122.1, 121.6, 119.3, 116.8 ppm.

### Synthetic Method 16 4-phenoxypyridine-2-carboxylic acid quinolin-8-ylamide (Example Compound 32)

To a stirred solution of phenol (0.067 g, 0.711 mmol) in 1-methyl-2-pyrrolidinone (5.0 mL) was added sodium hydride (0.030 g of 60% dispersion, 0.744 mmol). The reaction mixture was stirred at room temperature. After 5 minutes, example compound 22 (synthetic method 12; 0.192 g, 0.677 mmol) was added in one portion, and the resulting solution was heated to 85° C. After 16 hours, the brown mixture was allowed to cool to room temperature, and water (100 mL) was added. The suspension was extracted with ethyl acetate. The combined organic phases were washed with water and brine, dried (magnesium sulfate), and concentrated to provide crude product as a brown oily solid. Flash chromatography over silica (dichloromethane/methanol) afforded 0.177 g (77%) of product as an off-white solid: ¹H NMR (CDCl₃) δ 12.30 (br s, 1H), 8.97-8.95 (m, 2H), 8.63 (d, J=5.5 Hz, 1H), 8.18 (dd, J=8.2, 1.7 Hz, 1H), 7.86 (d, J=2.5 Hz, 1H), 7.61-7.54 (m, 2H), 7.50-7.44 (m, 3H), 7.31-7.27 (m, 1H), 7.16-7.14 (m, 2H), 7.05 (dd, J=5.5, 2.5 Hz, 1H) ppm. ¹³C NMR (CDCl₃) δ 166.4, 162.3, 153.8, 152.8, 150.2, 148.7, 139.3, 136.2, 134.4, 130.4, 128.1, 127.3, 125.8, 122.1, 121.6, 120.9, 116.8, 114.5, 110.5 ppm.

### Synthetic Method 17 3-benzyloxypyridine-2-carboxylic acid quinolin-8-ylamide (Example Compound 47)

To a stirred solution of example compound 46 (prepared according to synthetic method 12; 0.313 g, 1.18 mmol), benzyl alcohol (0.153 g, 1.42 mmol), and triphenylphosphine (0.402 g, 1.53 mmol) in chloroform (10 mL) was added diethyl azodicarboxylate (0.247 g, 1.42 mmol). The resulting bright yellow solution was stirred at room temperature. After 2 hours, the mixture was concentrated and the residue dissolved in a 1:1 dichloromethane/ethyl acetate mixture (50 mL). The solution was washed successively with aqueous sodium bicarbonate solution and brine, dried (magnesium sulfate) and concentrated. The resulting yellow solid was triturated with ethyl acetate to afford 0.128 g (31%) of product as a light yellow solid: ¹H NMR (CDCl₃) δ 12.00 (br s, 1H), 9.10-9.08 (m, 1H), 8.83 (dd, J=4.1, 1.7 Hz, 1H), 8.40 (dd, J=4.3, 1.4 Hz, 1H), 8.17 (dd, J=8.2, 1.7 Hz, 1H), 7.62-7.52 (m, 4H), 7.46-7.30 (m, 6H), 5.39 (s, 2H) ppm. ¹³C NMR (CDCl₃) δ 162.3, 155.2, 148.4, 141.0, 140.0, 139.2, 136.2, 136.0, 135.0, 128.7, 128.1, 127.5, 127.0, 126.9, 123.0, 121.6, 121.4, 116.8, 71.0 ppm.

### Synthetic Method 18 2-dimethylamino-N-quinolin-8-yl-acetamide (Example Compound 55)

### Step 1) Preparation of 2-Bromo-N-quinolin-8-yl-acetamide

To a stirred solution of 8-aminoquinoline (0.144 g, 0.999 mmol) and triethylamine (0.120 g, 1.19 mmol) in dichloromethane (5 ml) was added, dropwise, a solution of bromoacetylbromide (0.220 mg, 1.09 mmol) in dichloromethane (2 ml). After 1 hour the mixture was diluted with dichloromethane (20 ml) and washed with 1 N hydrochloric acid, aqueous sodium bicarbonate solution and brine. The organic phase was dried (sodium sulfate) and concentrated to provide 0.168 g (63%) of crude product which was used without further purification: ¹H NMR (CDCl₃) δ 10.70 (br s, 1H), 8.84 (d, 1H), 8.73 (m, 1H), 7.54 (m, 2H), 7.47 (m, 1H), 4.16 (s, 2H) ppm.

### Step 2) Preparation of 2-Dimethylamino-N-quinolin-8-yl-acetamide

To a stirred solution of 2-bromo-N-quinolin-8-yl-acetamide (0.160 g, 0.604 mmol) in tetrahydrofuran (2 ml) was added a 2.0 M solution of dimethylamine in tetrahydrofuran (2.0 ml, 4.0 mmol). After 18 hours the reaction was poured into water (25 ml) and extracted with ethyl acetate. The combined organic layers were washed with aqueous sodium bicarbonate solution and brine, dried (sodium sulfate) and concentrated. The resulting residue was purified by flash chromatography over silica (ethyl acetate/hexanes) to afford 0.080 g (58%) of product as an amber oil: ¹H NMR (CDCl₃) δ 11.18 (br s, 1H), 8.87 (d, 1H), 8.81 (d, 1H), 8.14.(d, 1H), 7.52 (m, 2H), 7.44 (m, 1H), 3.24 (s, 2H), 2.49 (s, 6H) ppm.

### Synthetic Method 19 (S)-pyrrolidine-2-carboxylic acid quinolin-8-ylamide (Example Compound 57)

### Step 1) Preparation of (S)-2-(Quinolin-8-ylcarbamoyl)-pyrrolidine-1-carboxylic acid t-butyl ester

To a stirred solution of 8-aminoquinoline (14.42 g, 100.0 mmol) and (S)-N-(tert-butoxycarbonyl)-proline (23.68 g, 0.110 mol) in dichloromethane (250 ml) was added 1-[3-(dimethylamino)propyl]-3-ethyl carbodiimide hydrochloride (21.09 g, 110.0 mmol) and catalytic 4-(dimethylamino)pyridine (ca. 200 mg). After 18 hours, the mixture was diluted with dichloromethane (100 ml) and washed with aqueous sodium bicarbonate and brine. The organic phase was dried (sodium sulfate) and concentrated to give a light green solid. Recrystallization from ethanol (2 crops) afforded 29.40 g (86%) of product as off-white needles.

### Step 2) Preparation of (S)-Pyrrolidine-2-carboxylic acid quinolin-8-ylamide

To a stirred suspension of the product of step 1 (29.30 g, 85.82 mmol) in 1,4-dioxane (75 ml) was added a solution of 4 N hydrogen chloride in dioxane (75 ml, 300 mmol). The solid rapidly dissolved and the reaction became a clear yellow solution. Stirring was continued for 2 hours, during which time a gummy solid formed. The reaction was made basic with the addition of a 4 M aqueous sodium hydroxide solution and extracted with successive portions of ethyl acetate. The combined organic layers were washed with brine, dried (sodium sulfate) and concentrated to afford 19.67 g (95%) of product as a viscous oil: ¹H NMR (CDCl₃) δ 11.55 (br s, 1H), 8.86 (dd, 1H), 8.82 (dd, 1H), 8.13 (dd, 1H), 7.50 (m, 2H), 7.43 (dd, 1H), 4.03 (m, 1H), 3.16 (m, 2H), 2.82 (br s, 1H), 2.27 (m, 1H), 2.12 (m, 1H), 1.81 (m, 2H) ppm.

### Synthetic Method 20 (S)-1-methyl-pyrrolidine-2-carboxylic acid quinolin-8-ylamide (Example Compound 58)

To a stirred solution of example compound 57 (synthetic method 19; 20.71 g, 85.83 mmol) in 1,2-dichloroethane (200 ml) was added a 37% aqueous formaldehyde solution (20 ml, 250 mmol) followed by sodium triacetoxyborohydride (portion-wise over 20 minutes; 55.00 g, 259.5 mmol). After 18 hours the reaction was poured into a 4 M aqueous sodium hydroxide solution (300 ml). The aqueous layer was separated and extracted with dichloromethane. The combined organic layers were washed with brine, dried (sodium sulfate) and concentrated. The resulting solid was recrystallized from ethanol/water to afford 16.81 g (76%) of product as a beige solid: ¹H NMR (CDCl₃) δ 11.33 (br s, 1H), 8.87 (m, 2H), 8.15 (d, 1H), 7.50 (m, 2H), 7.44 (m, 1H), 3.34 (t, 1H), 3.10 (m, 1H), 2.54 (s, 3H), 2.46 (m, 1H), 2.33 (m, 1H), 2.04 (m, 1H), 1.91 (m, 1H), 1.84 (m, 1H) ppm.

### Synthetic Method 21 pyridine-2-sulfonic acid (5,6-dimethyl-quinolin-8-yl)-amide (Example Compound 75)

To a stirred solution of 2-pyridine sulfonyl chloride (0.298 mg, 1.68 mmol) in dichloromethane (4 mL) was added 8-amino-5,6-dimethyl quinoline (prepared according to synthetic methods 1, 2; 0.124 g, 0.722 mmol) and catalytic 4-(dimethylamino)pyridine. After overnight stirring, the reaction was diluted with dichloromethane (˜30 mL) and washed with aqueous saturated sodium bicarbonate solution and water. The organic layer was dried (sodium sulfate) and concentrated to a yellow residue. Flash chromatography over silica (dichloromethane/methanol) afforded 0.130 g (42%) of product as a pale yellow solid: ¹H NMR (CDCl₃) δ 9.20-9.70 (br s, 1H), 8.72 (d, 1H), 8.55 (d, 1H), 8.31 (d, 1H), 8.08 (d, 1H), 7.88-7.69 (m, 2H), 7.32-7.47 (m, 2H), 2.46 (s, 3H), 2.44 (s, 3H) ppm.

### Synthetic Method 22 pyridine-2-carboxylic acid quinolin-8-ylamide hydrochloride salt (Example Compound 93)

To a stirred solution of example compound 1 (prepared according to synthetic method 12; 2.00 g, 8.02 mmol) in chloroform (50 mL) was added, dropwise, a 2.0 M solution of hydrogen chloride in diethyl ether (4.0 mL, 8.0 mmol). The resulting precipitate was filtered off under nitrogen, rinsed with chloroform and vacuum oven dried to afford 1.54 g (67%) of product as a beige solid: ¹H NMR (CDCl₃) δ 12.13 (s, 1H), 9.01 (dd, J=4.2, 1.3 Hz, 1H), 8.88 (d, J=7.6 Hz, 1H), 8.83 (d, J=4.2 Hz, 1H), 8.45 (dd, J=8.3, 1.3 Hz, 1H), 8.25 (d, J=7.7 Hz, 1H), 8.15-8.07 (m, 1H), 7.77-7.62 (m, 4H), 6.56 (br s, 1H) ppm.

### Synthetic Method 23 (S)-1-methyl-pyrrolidine-2-carboxylic acid quinolin-8-ylamide L-tartrate salt (Example Compound 108)

To a stirred solution of L-tartaric acid (10.87 g, 72.42 mmol) in acetone (250 ml) was added, dropwise, a solution of example compound 58 (synthetic method 20; 16.81 g, 69.67 mmol) in acetone (100 ml). The resulting precipitate was filtered, washed with acetone and vacuum oven dried to afford 26.31 g (98%) of product as an off-white solid: ¹H NMR (D₂O) δ 8.71 (d, 1H), 8.27 (d, 1H), 7.99 (d, 1H), 7.69 (d, 1H), 7.48 (m, 2H), 4.43 (t, 1H), 4.33 (s, 2H), 3.70 (m, 1H), 3.19 (q, 1H), 2.91 (s, 3H), 2.65 (m, 1H), 2.18 (m, 2H), 2.01 (m, 1H) ppm.

### Synthetic Method 24 4-(4-methylpiperazin-1-yl)pyridine-2-carboxylic acid quinolin-8-ylamide trifluoroacetate salt (Example Compound 100)

To a stirred solution of example compound 34 (synthetic method 14; 0.095 g, 0.273 mmol) in diethyl ether (3.0 mL) and dichloromethane (1.0 mL) was added trifluoroacetic acid (0.031 g, 0.273 mmol). The resulting precipitate was isolated by filtration, washed with a small amount of diethyl ether and dried to provide 0.102 g (81%) of product as a white solid: ¹H NMR (CDCl₃) δ 12.30 (br s, 1H), 8.97-8.95 (m, 2H), 8.51 (d, J=5.8 Hz, 1H), 8.18 (dd, J=8.2, 1.7 Hz, 1H), 7.77 (d, J=2.6 Hz, 1H), 7.62-7.56 (m, 2H), 7.48 (dd, J=8.2, 4.1 Hz, 1H), 6.81 (dd, J=5.8, 2.7 Hz, 1H), 4.01-2.92 (m, 8H), 2.84 (s, 3H) ppm. ¹³C NMR (CDCl₃) δ 163.6, 163.2, 162.9, 162.7, 162.5, 154.7, 151.7, 149.8, 148.8, 139.3, 136.2, 134.3, 128.1, 127.2, 122.1, 121.7, 120.8, 117.9, 116.7, 115.0, 112.1, 110.6, 107.5, 52.5, 43.6, 43.4 ppm.

TABLE 1
Quinoline Substituted Pyridine-2-carboxylic acid quinolin-8-ylamides

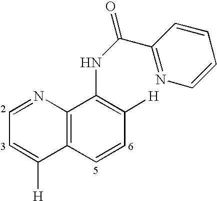

| Cmpd | Method | | | | |
|---|---|---|---|---|---|
| No. | Used | 2 | 3 | 5 | 6 |
| 1 | 12 | H | H | H | H |
| 2 | 12 | H | H | H | OCH₃ |
| 3 | 12 | H | H | OCH₃ | H |
| 4 | 1,2,12 | H | H | H | Cl |
| 5 | 1,3,12 | H | H | H | CF₃ |
| 6 | 12 | CH₃ | H | H | H |
| 7 | 12 | H | Cl | H | H |
| 8 | 1,3,12 | H | H | H | OCH₂CH₃ |
| 9 | 4,2,12 | H | H | 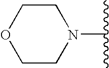 | H |
| 10 | 1,5,3,12 | H | H | H | 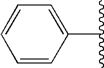 |
| 11 | 12 | H | H | H | CH₃ |
| 12 | 6,2,12 | H | H | 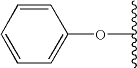 | H |
| 13 | 7,1,2,12 | H | H | H | 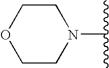 |
| 14 | 12 | H | H | CH₃ | H |
| 15 | 1,2,12 | H | H | Cl | Cl |
| 16 | 12,13 | H | H | Br | CH₃ |
| 17 | 1,2,12 | H | H | CH₃ | CH₃ |
| 18 | 12 | H | H | Cl | H |
| 19 | 4,8,12 | H | H | 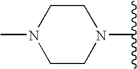 | H |
| 20 | 9,10,2,12 | H | H | H | 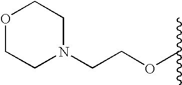 |
| 21 | 4,8,12 | H | H | 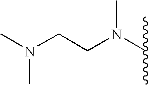 | H |

TABLE 2
Pyridine Substituted Pyridine-2-carboxylic acid quinolin-8-ylamides

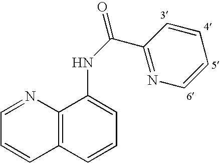

| Cmpd | Method | | | | |
|---|---|---|---|---|---|
| No. | Used | 3′ | 4′ | 5′ | 6′ |
| 22 | 12 | H | Cl | H | H |
| 23 | 12 | —OCH₂CH₂CH₃ | H | H | H |
| 24 | 12 | CH₃ | H | H | H |
| 25 | 12 | H | H | H | CH₃ |
| 26 | 12 | H | H | H | Br |
| 27 | 12 | H | 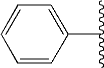 | H | H |
| 28 | 12 | H | —C₄H₄— | | H |
| 29 | 12 | H | —OCH₃ | H | H |
| 30 | 12,14 | H | 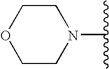 | H | H |
| 31 | 12,15 | H | 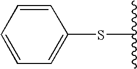 | H | H |
| 32 | 12,16 | H | 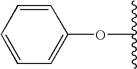 | H | H |
| 33 | 12 | H | H | 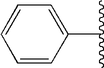 | H |
| 34 | 12,14 | H | 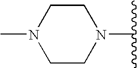 | H | H |
| 35 | 12,14 | H | 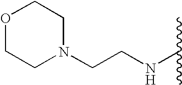 | H | H |
| 36 | 12,14 | H | 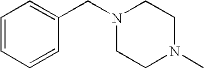 | H | H |
| 37 | 12,14 | H | 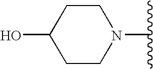 | H | H |
| 38 | 12,14 | H | 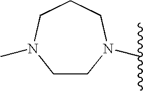 | H | H |
| 39 | 12,14 | H | 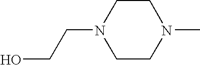 | H | H |
| 40 | 12,14 | H | 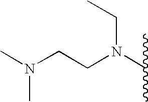 | H | H |
| 41 | 12,14 | H | 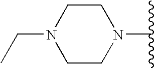 | H | H |
| 42 | 12,14 | H | 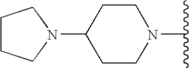 | H | H |
| 43 | 12,14 | H | 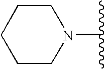 | H | H |
| 44 | 12,14 | H | 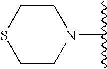 | H | H |
| 45 | 12,15 | H | 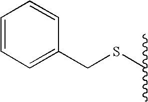 | H | H |
| 46 | 12 | OH | H | H | H |
| 47 | 12,17 | 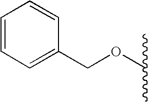 | H | H | H |
| 48 | 12,17 | 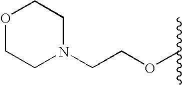 | H | H | H |
| 49 | 12,17 | 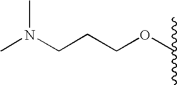 | H | H | H |

TABLE 3
Quinoline/Pyridine Disubstituted
Pyridine-2-carboxylic acid quinolin-8-ylamides

| Cmpd | Method | |
|---|---|---|
| No. | Used | Structure |
| 50 | 4,2,12 | 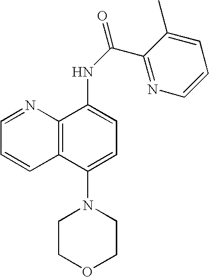 |
| 51 | 4,2,12 | 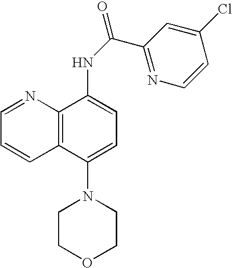 |
| 52 | 4,2,12 | 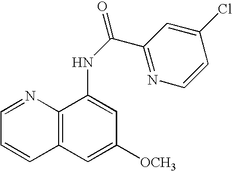 |
| 53 | 12,14 | 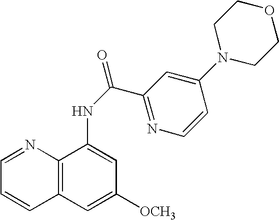 |
| 54 | 12,14 | 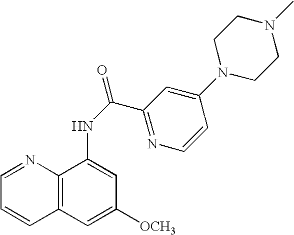 |

TABLE 4
α-Amino-N-quinolin-8-yl Acetamides

| Cmpd | Method | |
|---|---|---|
| No. | Used | Structure |
| 55 | 18 | 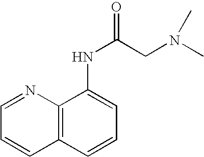 |
| 56 | 18 | 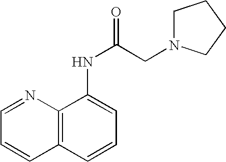 |
| 57 | 19 | 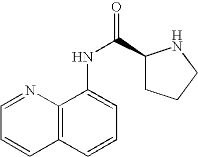 |
| 58 | 19,20 | 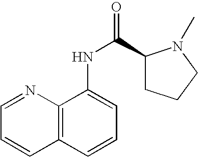 |
| 59 | 12 | 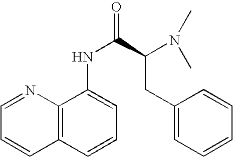 |
| 60 | 12 | 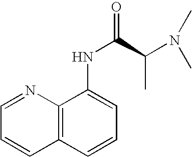 |
| 61 | 19,20 | 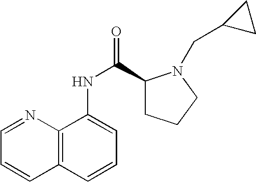 |
| 62 | 19,20 | 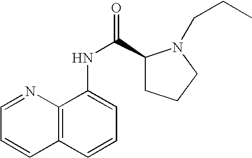 |
| 63 | 19,20 | 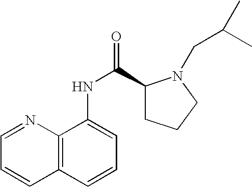 |
| 64 | 19,20 | 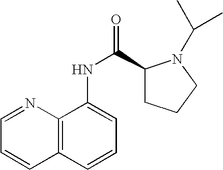 |
| 65 | 19,20 | 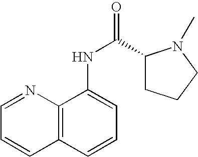 |
| 66 | 12 | 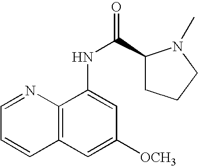 |
| 67 | 12 | 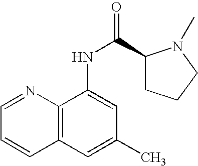 |
| 68 | 4,2,12 | 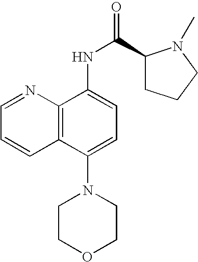 |
| 69 | 19,20 | 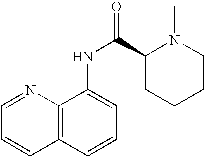 |
| 70 | 19,20 | 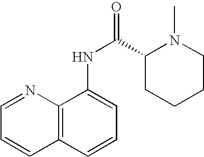 |

TABLE 5
Pyridine-2-sulfonic acid quinolin-8-ylamides

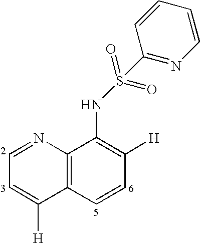

| Cmpd | Method | | | | |
|---|---|---|---|---|---|
| No. | Used | 2 | 3 | 5 | 6 |
| 71 | 21 | H | H | H | H |
| 72 | 21 | H | H | H | OCH₃ |
| 73 | 1,2,21 | H | H | Cl | Cl |
| 74 | 21 | H | H | CH₃ | H |
| 75 | 1,2,21 | H | H | CH₃ | CH₃ |

TABLE 6
Amino Cinnoline/Quinoxaline Amides

| Cmpd | Method | |
|---|---|---|
| No. | Used | Structure |
| 76 | 12 | 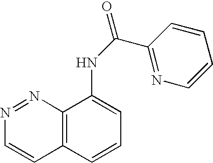 |
| 77 | 12 | 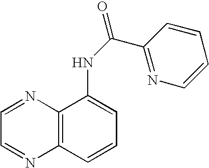 |
| 78 | 21 | 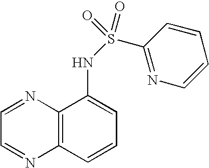 |

TABLE 7
Substituted Pyridine-2-carboxylic acid phenyl amides

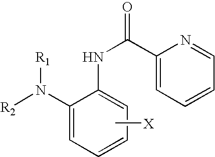

| | | | | |
|---|---|---|---|---|
| Cmpd | Method | | | |
| No. | Used | R1 | R2 | X |

| | | | |
|---|---|---|---|
| 79 | 11,12 | —(CH₂)₄— | H |

| | | | | |
|---|---|---|---|---|
| 80 | 11,12 | CH₃ | CH₃ | H |
| 81 | 11,12 | CH₃ | Et | H |
| 82 | 11,12 | CH₃ | Pr | H |
| 83 | 11,12 | CH₃ | i-Pr | H |
| 84 | 11,12 | Et | Et | H |
| 85 | 11,12 | CH₃ | CH₃ | 3-CF₃ |
| 86 | 11,12 | CH₃ | Et | 3-CF₃ |
| 87 | 11,12 | CH₃ | CH₃ | 4-CH₃ |
| 88 | 11,12 | CH₃ | Et | 4-CH₃ |
| 89 | 11,12 | CH₃ | CH₃ | 4-OCH₃ |
| 90 | 11,12 | CH₃ | Et | 4-OCH₃ |
| 91 | 11,12 | CH₃ | CH₃ | 3,4-Cl₂ |
| 92 | 11,12 | CH₃ | Et | 3,4-Cl₂ |

TABLE 8
Salt Forms

| Cmpd | Method | | |
|---|---|---|---|
| No. | Used | Structure | Salt |
| 93 | 12,22 | 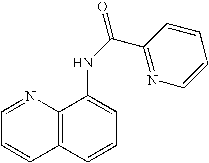 | HCl |
| 94 | 12,22 | 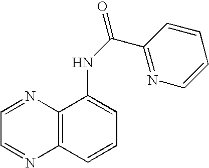 | HCl |
| 95 | 1,2,12,22 | 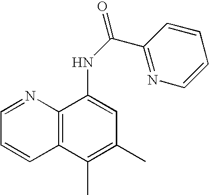 | HCl |
| 96 | 12,22 | 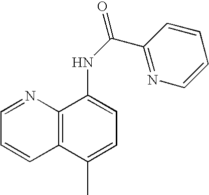 | HCl |
| 97 | 1,2,12,22 | 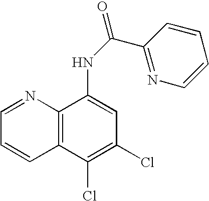 | HCl |
| 98 | 4,8,12,23 | 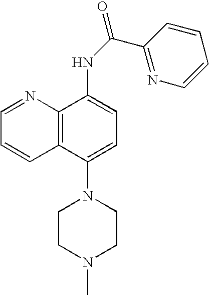 | Tartrate |
| 99 | 9,10,2,12,23 | 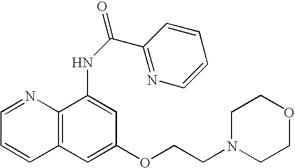 | Tartrate |
| 100 | 12,14,24 | 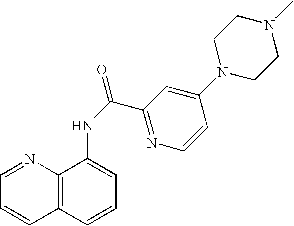 | CF₃CO₂H |
| 101 | 12,14,23 | 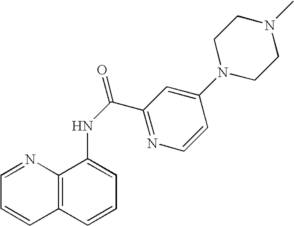 | Tartrate |
| 102 | 12,14,23 | 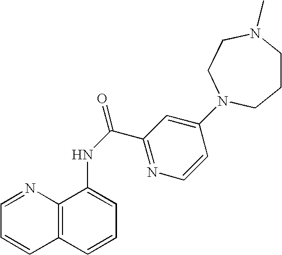 | Tartrate |
| 103 | 12,14,24 | 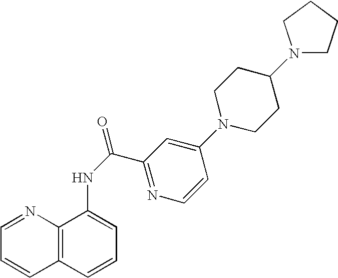 | CF₃CO₂H |
| 104 | 12,17,24 | 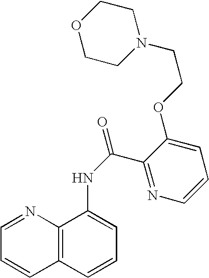 | CF₃CO₂H |
| 105 | 12,14,23 | 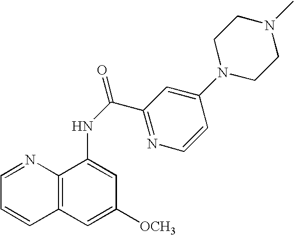 | Tartrate |
| 106 | 11,12,22 | 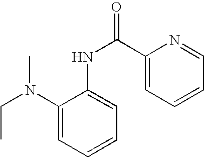 | HCl |
| 107 | 19,20,22 | 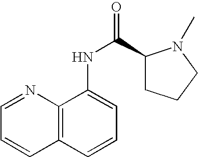 | HCl |
| 108 | 19,20,23 | 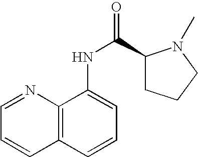 | Tartrate |
| 109 | 1,2,21,22 | 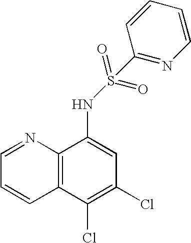 | HCl |
| 110 | 4,8,21,23 | 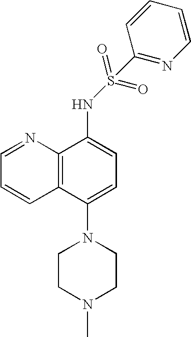 | Tartrate |

### EXAMPLES OF BIOLOGICAL DATA

VEGF and b-FGF Driven HMVEC Proliferation Screens

HMVEC cells were plated on collagen-coated plates at 2500 cells/well for 24 hours. Various concentrations of test compounds were then added to duplicate plates. 0.1 to 1% DMSO was added to enhance the solubility of the test compounds, as needed. Anti-FGF antibody (final concentration of 5 μg/mL) was added to four control wells as a standard inhibitor. At the same time the compounds were added, a T-zero plate was generated (Alamar blue was added to a plate and read after three hours). The plates with the compounds were incubated for one hour at 37° C., 5% carbon dioxide. At this point, b-FGF was added (final concentration of 2.5 ng/mL). Tween-20 (10 μL) was added to four additional control wells to represent total death and the plates were returned to the carbon dioxide incubator for 6 days. On day 6 Alamar blue was added and the plates read after three hours.

The assay was run identically using VEGF in place of b-FGF. The final concentration of VEGF 165, in this mode, was 10 ng/mL. The control inhibitor was anti-VEGF antibody (final concentration of 2 pg/mL). All other conditions were the same.

Dermal Fibroblast Toxicity Counterscreen

In parallel with the VEGF and FGF screens, a normal cell toxicity assay was run. Primary Human Dermal Fibroblasts (Clonetics) were plated at 5000 cells/well. After 24 hours various concentrations of test compounds, were added in duplicate plates and 10% Tween-20 was added to four control wells to represent total death. At the same time a T-zero plate was generated. The plates were returned to the carbon dioxide incubator and on day 6 Alamar blue was added and the plates read after 3 hours.

Using a T-zero plate allows the observation of compounds that cause the cells to become static as well as inhibit growth. The ideal compounds will exhibit potent inhibition of HMVEC proliferation in the presence of b-FGF and/or VEGF while not inhibiting normal cell growth.

TABLE 9
In Vitro Activity of Example Compounds: VEGF and b-FGF Stimulated
HMVEC Proliferation Assay versus Dermal Fibroblast Toxicity Assay

| Compound | VEGF | b-FGF | D. Fibroblast |
|---|---|---|---|
| Number | LC₅₀ (μM) | LC₅₀ (μM) | LC₅₀ (μM) |
| 1 | 0.065 | 0.061 | >10 |
| 2 | 0.038 | 0.031 | 3.4 |
| 3 | 0.12 | 0.11 | >10 |
| 4 | 0.12 | 0.11 | >10 |
| 5 | 0.16 | 0.12 | >10 |
| 6 | 3.9 | 3.2 | >10 |
| 7 | 0.31 | 0.32 | >10 |
| 8 | 0.096 | 0.081 | >10 |
| 9 | 0.035 | 0.037 | >10 |
| 10 | 0.26 | 0.38 | >10 |
| 11 | 0.073 | 0.068 | >10 |
| 12 | 1.0 | 1.0 | >10 |
| 13 | 0.025 | 0.026 | >10 |
| 14 | 0.13 | 0.17 | >10 |
| 15 | 0.21 | 0.22 | >10 |
| 16 | 0.38 | 0.38 | >10 |
| 17 | 0.16 | 0.13 | >10 |
| 18 | 0.17 | 0.20 | >10 |
| 19 | 0.028 | 0.028 | >10 |
| 20 | 0.030 | 0.031 | >10 |
| 21 | 3.7 | 2.4 | >10 |
| 22 | 0.10 | 0.13 | >10 |
| 23 | 0.18 | 0.21 | >10 |
| 24 | 0.14 | 0.15 | >10 |
| 25 | 2.5 | 2.3 | >10 |
| 26 | 0.086 | 0.12 | >10 |
| 27 | 0.64 | 0.95 | >10 |
| 28 | 0.48 | 0.61 | >10 |
| 29 | 0.060 | 0.069 | >10 |
| 30 | 0.022 | 0.026 | 8.0 |
| 31 | 0.17 | 0.15 | >10 |
| 32 | 1.1 | 1.8 | >10 |
| 33 | 4.7 | 6.1 | >10 |
| 34 | 0.013 | 0.018 | >10 |
| 35 | 0.12 | 0.16 | >10 |
| 36 | 0.84 | 0.90 | >10 |
| 37 | 0.29 | 0.31 | >10 |
| 38 | 0.026 | 0.025 | >10 |
| 39 | 0.22 | 0.65 | >10 |
| 40 | 0.016 | 0.021 | 7.6 |
| 41 | 0.017 | 0.018 | 4.2 |
| 42 | 0.042 | 0.033 | 3.6 |
| 43 | 0.40 | 0.22 | >10 |
| 44 | 0.18 | 0.25 | >10 |
| 45 | 0.061 | 0.19 | >10 |
| 46 | 0.051 | 0.066 | >10 |
| 47 | 0.28 | 0.40 | >10 |
| 48 | 0.95 | 1.6 | >10 |
| 49 | 1.7 | 2.5 | >10 |
| 50 | 0.11 | 0.074 | >10 |
| 51 | 0.22 | 0.23 | >10 |
| 52 | 0.093 | 0.085 | >10 |
| 53 | 0.030 | 0.028 | >10 |
| 54 | 0.015 | 0.020 | >10 |
| 55 | 0.63 | 0.59 | >10 |
| 56 | 1.3 | 1.4 | >10 |
| 57 | 1.3 | 1.2 | >10 |
| 58 | 0.19 | 0.17 | >10 |
| 59 | 1.2 | 1.1 | >10 |
| 60 | 0.73 | 0.52 | >10 |
| 61 | 1.5 | 1.5 | >10 |
| 62 | 1.3 | 1.2 | >10 |
| 63 | 5.5 | 4.4 | >10 |
| 64 | 6.4 | 5.2 | >10 |
| 65 | 0.23 | 0.29 | >10 |
| 66 | 0.24 | 0.24 | >10 |
| 67 | 0.23 | 0.35 | >10 |
| 68 | 0.43 | 0.82 | >10 |
| 69 | 3.1 | 2.6 | >10 |
| 70 | 3.9 | 2.8 | >10 |
| 71 | 0.12 | 0.11 | 7.0 |
| 72 | 0.17 | 0.24 | >10 |
| 73 | 1.1 | 1.0 | >10 |
| 74 | 0.12 | 0.11 | >10 |
| 75 | 0.19 | 0.16 | >10 |
| 76 | 3.5 | 3.3 | >10 |
| 77 | 0.17 | 0.18 | >10 |
| 78 | 2.4 | 4.2 | >10 |
| 79 | 8.0 | 6.5 | >10 |
| 80 | 0.77 | 0.83 | >10 |
| 81 | 0.62 | 0.50 | >10 |
| 82 | 2.0 | 1.3 | >10 |
| 83 | 9.6 | 6.5 | >10 |
| 84 | 1.6 | 1.5 | >10 |
| 85 | 6.1 | 1.2 | >10 |
| 86 | 6.0 | 2.4 | >10 |
| 87 | 0.63 | 0.59 | >10 |
| 88 | 0.72 | 0.68 | >10 |
| 89 | 1.7 | 1.2 | >10 |
| 90 | 1.8 | 1.6 | >10 |
| 91 | 3.5 | 2.4 | >10 |
| 92 | 8.2 | 7.9 | >10 |
| 93 | 0.072 | 0.071 | >10 |
| 94 | 0.28 | 0.29 | >10 |
| 95 | 0.15 | 0.17 | >10 |
| 96 | 0.14 | 0.10 | >10 |
| 97 | 0.25 | 0.25 | >10 |
| 98 | 0.033 | 0.036 | >10 |
| 99 | 0.028 | 0.031 | >10 |
| 100 | 0.018 | 0.019 | 2.9 |
| 101 | 0.015 | 0.014 | >10 |
| 102 | 0.024 | 0.031 | >10 |
| 103 | 0.033 | 0.038 | 4.3 |
| 104 | 0.81 | 1.4 | >10 |
| 105 | 0.091 | 0.12 | 9.6 |
| 106 | 1.5 | 1.2 | >10 |
| 107 | 0.46 | 0.41 | >10 |
| 108 | 0.29 | 0.22 | >10 |
| 109 | 0.45 | 0.34 | >10 |
| 110 | 1.5 | 1.3 | >10 |

### Compound 9 And Compound 98 are Effective at Preventing Neovascularization in a Mouse Model of Oxygen Induced Retinopathy

Seven day old neonatal C57BL/6 mouse pups and their nursing dam were placed in an isobaric chamber that maintains a 75% oxygen level. The pups were returned to a normoxic environment at 12 days of age. The test articles and vehicle controls were administered intravitreally (once on day 12). Approximately one half of the pups from each litter were injected, the remaining pups were used as controls. Pups were euthanized on day 17 and their left/treated eye is processed for histological sectioning.

The OIR model resulted in neovascularization that occurred on the apical surface of the retina. The degree of neovascularization was measured by examining serial sections of the eye taken at 100 micron intervals. The numbers of endothelial nuclei that were internal but contiguous to the inner limiting membrane were counted. The number of nuclei from test article treated animals was compared with those counted in control animals.

| Test Article | Route | Dose |
|---|---|---|
| DMSO vehicle control | IVT | |
| 20% Hydroxypropyl β- | IVT | |
| cyclodextrin vehicle control | | |
| Compound 9 in DMSO | IVT | 30 mg/ml |
| Compound 98 in 20% | IVT | 30 mg/ml |
| hydroxypropyl β- | | |
| cyclodextrin | | |

Intravitreal Administrations:

Administration of the test article resulted in an immediate clouding of the vitreous that extended into the anterior chamber. This was not observed in the DMSO treated animals. The pupils of the Compound 9 and the Compound 98 animals were markedly dilated for the duration of the study.

Study Animals

| | | Number Treated |
|---|---|---|
| Test Article | Litter I.D. | of Litter Total |
| DMSO control | 1650 | 3 of 3 |
| | 1651 | 4 of 7 |
| Compound 9 | 1645 | 5 of 9 |
| | 1649 | 4 of 6 |
| Compound 98 | 1644 | 5 of 9 |
| | 1648 | 4 of 7 |

There was no detectable difference in the level of neovascularization between the eyes injected with DMSO and the eyes naive to treatment.

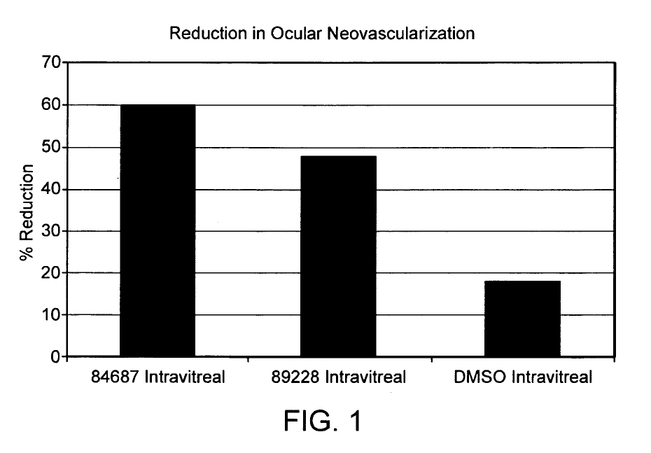

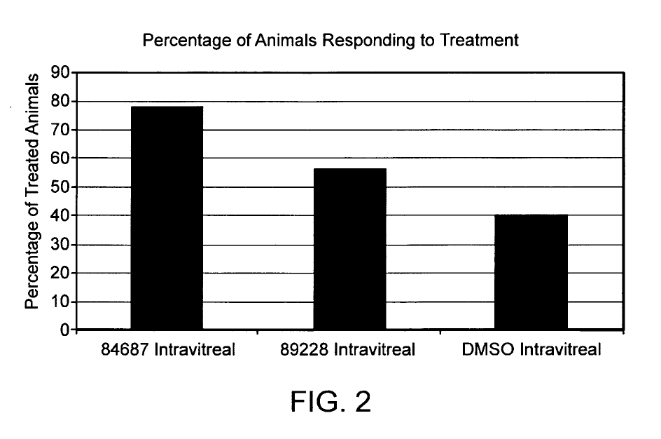

The intravitreal administration of both Compound 9 and Compound 98 resulted in a decrease in the amount of neovascularization observed. A greater effect was observed with Compound 9. This difference is observed when the average amounts of neovascular events for the treated eyes are compared with the controls (FIG. 1). The difference is also observed when the tallying the number of individual responders to treatment (FIG. 2).

1. A compound represented by the following formula: 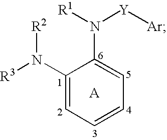
or a pharmaceutically acceptable salt thereof, wherein:
Ring A is optionally substituted at any one or more substitutable ring carbon atoms;
—Y— is —SO₂— or —C(O)—;
Ar is an optionally substituted monocyclic aryl group;
R¹ is H or a C1-C6 alkyl group optionally substituted with amino, hydroxyl, methoxy or ethoxy;
R² and R³ are independently C1-C6 alkyl optionally substituted with amino, hydroxyl, methoxy or ethoxy; or
R₂ is absent and R₃, taken together with the nitrogen atom to which it is bonded and C1 and C2 of Ring A, forms an optionally substituted six membered nitrogen-containing aromatic group referred to as “Ring B”; and
wherein each substituent on Ring A, B and Ar is independently selected;
provided that when Ar is pyridyl, then at least one of Ring A, Ring B and Ar is substituted with a group other than alkyl,
provided that when Ar is phenyl or pyridyl, then Ar is not substituted with a carboxamide.

2. The compound of claim 1 wherein the compound is represented by the following formula: 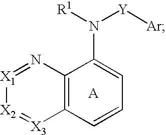
or a pharmaceutically acceptable salt thereof, wherein:
Ring A is optionally substituted at any substituted ring carbon atom;
X₁—X₃ are independently N, CH or CR⁶, provided that X₁ and X₂ are not both N;
each R⁶ is independently halogen, alkyl, haloalkyl, —OR²⁰, —O(haloalkyl), —SR²⁰, —NO₂, —CN, —N(R²¹)₂, —NR²¹C(O)R²⁰, —NR²¹CO₂R²², —N(R²¹)C(O)N(R²¹)₂, —C(O)R²⁰, —CO₂R²⁰, —C(O)N(R²¹)₂, —S(O)₂R²⁰, —SO₂N(R²¹)₂, —S(O)R²², —NR²¹SO₂N(R²¹)₂, —NR²¹SO₂R²², —Vₒ—N(R²¹)₂, —O—Vₒₒ—N(R²¹)₂, —S—Vₒₒ—N(R²¹)₂ or —N(R²¹)—Vₒₒ—N(R²¹)₂;
each Vₒ is independently a C1-C4 alkylene group;
each Vₒₒ is independently a C2-C4 alkylene group;
each R²⁰ is independently hydrogen or an alkyl group; and
each R²¹ is independently hydrogen, an alkyl group, —CO₂R²¹, —SO₂R²¹ or —C(O)R²⁰; or —N(R²¹)₂ is an optionally substituted non-aromatic nitrogen-containing heterocyclic group; and
each R²² is independently an alkyl group.

3. The compound of claim 2 wherein Ar is an optionally substituted monocyclic nitrogen-containing aromatic group.

4. The compound of claim 3 wherein Ar is an optionally substituted pyridyl group.

5. The compound of claim 4 wherein the compound is represented by the following formula: 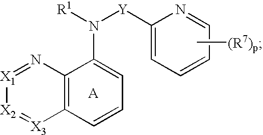
or a pharmaceutically acceptable salt thereof, wherein:
Ring A is optionally substituted at any one or more substitutable rings carbon atoms;
p is 0, 1, 2, 3 or 4;
each R⁷ is independently halogen, alkyl, haloalkyl, Ar¹, —OR³⁰, —O(haloalkyl), —SR³⁰, —NO₂, —CN, —N(R³¹)₂, —NR³⁰C(O)R³⁰, —NR³¹CO₂R³², —N(R³¹)C(O)N(R³¹)₂, —C(O)R³⁰, —CO₂R³⁰, —S(O)₂R³⁰, —SO₂N(R³¹)₂, —S(O)R³², —NR³¹SO₂N(R³¹)₂, —NR³¹SO₂R³², —V—Ar¹, —V—OR³⁰, —V—O(haloalkyl), —V—SR³⁰, —V—NO₂, —V—CN, —V—N(R³¹)₂, —V—NR³¹C(O)R³⁰, —V—NR³¹CO₂R³², —V—N(R³¹)C(O)N(R³¹)₂, —V—C(O)R³⁰, —V—CO₂R³⁰, —V—S(O)₂R³⁰, —V—SO₂N(R³¹)₂, —V—S(O)R³², —V—NR³¹SO₂N(R³¹)₂, —V—NR³¹SO₂R³², —O—V—Ar¹, —O—V₁—N(R³¹)₂, —S—V—Ar¹, —S—V₁—N(R³¹)₂, —N(R³¹)—V₁—Ar¹, —N(R³¹)—V₁—N(R³¹)₂, —NR³¹C(O)—V—N(R³¹)₂, —NR³¹C(O)—V—Ar¹, —C(O)—V—N(R³¹)₂, —C(O)—V—Ar¹, —CO₂—V₁—N(R³¹)₂, —CO₂—V—Ar¹, —C(O)N(R³¹)—V₁—N(R³¹)₂, —C(O)N(R³¹)—V—Ar¹, —S(O)₂—V—N(R³¹)₂, —S(O)₂—V—Ar¹, —SO₂N(R³¹)—V₁—N(R³¹)₂, —SO₂N(R³¹)—V—Ar¹, —S(O)—V—N(R³¹)₂, —S(O)—V—Ar¹, —NR³¹SO₂—V—N(R³¹)₂ or —NR³¹SO₂—V—Ar¹; or two adjacent R⁷ groups, taken together, form a methylenedioxy, ethylenedioxy or —[CH₂]₄— group; and
each V is independently a C1-C4 alkylene group;
each V₁ is independently a C2-C4 alkylene group;
Ar¹ is a monocyclic aromatic group each substituted with zero, one or two groups independently selected from halogen, alkyl, amino, alkylamino, dialkylamino, alkoxy, nitro, cyano, hydroxy, haloalkoxy or haloalkyl;
each R³⁰ is independently
i) hydrogen;
ii) an aromatic group substituted with zero, one or two groups represented by halogen, alkyl, amino, alkylamino, dialkylamino, alkoxy, nitro, cyano, hydroxy, haloalkoxy or haloalkyl; or
iii) an alkyl group optionally substituted with halogen, hydroxyl, alkoxy, nitro, cyano, alkoxycarbonyl, alkylcarbonyl or haloalkoxy; and
each R³¹ is independently R³⁰, —CO₂R³⁰, —SO₂R³⁰ or —C(O)R³⁰; or —N(R³¹)₂ taken together is an optionally substituted non-aromatic heterocyclic group; and
each R³² is independently:
i) an aromatic group substituted with zero, one or two groups represented by halogen, alkyl, amino, alkylamino, dialkylamino, alkoxy, nitro, cyano, hydroxy, haloalkoxy or haloalkyl; or
ii) an alkyl group optionally substituted with halogen, hydroxyl, alkoxy, nitro, cyano, alkoxycarbonyl, alkylcarbonyl or haloalkoxy.

6. The compound of claim 5 wherein the compound is represented by the following formula: 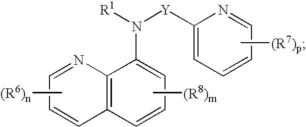
or a pharmaceutically acceptable salt thereof, wherein:
n and m are independently 0, 1, 2 or 3;
each R⁸ is independently:
i) halogen, alkyl, haloalkyl, Ar², —OR⁴⁰, —O(haloalkyl), —SR⁴⁰, —NO₂, —CN, —N(R⁴¹)₂, —NR⁴¹C(O)R⁴⁰, —NR⁴¹CO₂R⁴², —N(R⁴¹)C(O)N(R⁴¹)₂, —C(O)R⁴⁰, —
CO₂R⁴⁰, —C(O)N(R⁴¹)₂, —S(O)₂R⁴⁰, —SO₂N(R⁴¹)₂, —S(O)R⁴², —NR⁴¹SO₂N(R⁴¹)₂, —NR⁴¹SO₂R⁴², —V₂—Ar², —V₂—OR⁴⁰, —V₂—O(haloalkyl), —V₂—SR⁴⁰, —V₂—NO₂, —V₂—CN, —V₂—N(R⁴¹)₂, —V₂—NR⁴¹C(O)R⁴⁰, —V₂—NR⁴¹CO₂R⁴², —V₂—N(R⁴¹)C(O)N(R⁴¹)₂, —V₂—C(O)R⁴¹, —V₂—CO₂R⁴⁰, —V₂—C(O)N(R⁴¹)₂, —V₂—S(O)₂R⁴⁰, —V₂—SO₂N(R⁴¹)₂, —V₂—S(O)R⁴², —V₂—NR⁴¹SO₂N(R⁴¹)₂, —V₂—NR⁴¹SO₂R⁴², —O—V₂—Ar², —O—V₃—N(R⁴¹)₂, —S—V₂—Ar², —S—V₃—N(R⁴¹)₂, —N(R⁴¹)—V₂—Ar², —N(R⁴¹)—V₃—N(R⁴¹)₂, —NR⁴¹C(O)—V₂—N(R⁴¹)₂, —NR⁴¹C(O)—V₂—Ar², —C(O)—V₂—N(R⁴¹)₂, —C(O)—V₂—Ar², —CO₂—V₂—N(R⁴¹)₂, —CO₂—V₂—Ar², —C(O)N(R⁴¹)—V₃—N(R⁴¹)₂, —C(O)N(R⁴¹)—V₂—Ar², —S(O)₂—V₂—N(R⁴¹)₂, —S(O)₂—V₂—Ar², —SO₂N(R⁴¹)—V₃—N(R⁴¹)₂, —SO₂N(R⁴¹)—V₂—Ar², —S(O)—V₂—N(R⁴¹)₂, —S(O)—V₂—Ar², —NR⁴¹SO₂—V₂—N(R⁴¹)₂ or —NR⁴¹SO₂—V₂—Ar²; or
ii) two adjacent R⁸ groups, taken together, form a methylenedioxy, ethylenedioxy or —[CH₂]₄— group; and
each V₂ is independently a C1-C4 alkylene group;
each V₃ is independently a C2-C4 alkylene group;
Ar² is a monocyclic aromatic group substituted with zero, one or two groups independently selected from halogen, alkyl, amino, alkylamino, dialkylamino, alkoxy, nitro, cyano, hydroxy, haloalkoxy or haloalkyl;
each R⁴⁰ is independently:
i) hydrogen;
ii) an aromatic substituted with zero, one or two groups independently selected from halogen, alkyl, amino, alkylamino, dialkylamino, alkoxy, nitro, cyano, hydroxy, haloalkoxy or haloalkyl; or
iii) an alkyl group optionally substituted with halogen, hydroxyl, alkoxy, nitro, cyano, alkoxycarbonyl, alkylcarbonyl or haloalkoxy; and
each R⁴¹ is independently R⁴⁰, —CO₂R⁴⁰, —SO₂R⁴⁰ or —C(O)R⁴⁰; or —N(R⁴¹)₂ taken togther is an optionally substituted non-aromatic heterocyclic group; and
each R⁴² is independently:
i) an aromatic substituted with zero, one or two groups independently selected from halogen, alkyl, amino, alkylamino, dialkylamino, alkoxy, nitro, cyano, hydroxy, haloalkoxy or haloalkyl; or
ii) an alkyl group optionally substituted with halogen, hydroxyl, alkoxy, nitro, cyano, alkoxycarbonyl, alkylcarbonyl or haloalkoxy.

7. The compound of claim 6 wherein:
each R⁷ is independently selected from halogen, C1-C4 alkyl, C1-C4 haloalkyl, cyano, —OR³⁰, —SR³⁰, —N(R³¹)₂, Ar¹, —V—OR³⁰, —V—N(R⁴¹)₂, —V—Ar¹, —O—V—Ar¹, —O—V₁—N(R³¹)₂, —S—V—Ar¹, —S—V₁—N(R³¹)₂, —N(R³¹)—V—Ar¹ or —N(R³¹)—V₁—N(R³¹)₂;
each R⁸ is independently selected from halogen, C1-C4 alkyl, C1-C4 haloalkyl, cyano, —OR⁴⁰, —SR⁴⁰, —N(R⁴¹)₂, Ar², —V₂—OR⁴⁰, —V₂—N(R⁴¹)₂, —V₂—Ar², —O—V₂—Ar², —O—V₃—N(R⁴¹)₂, —S—V₂—Ar², —S—V₃—N(R⁴¹)₂, —N(R⁴¹)—V₂—Ar² or —N(R⁴¹)—V₃—N(R⁴¹)₂;
Ar¹ and Ar² are phenyl, each independently substituted with zero, one or two groups independently selected from halogen, alkyl, amino, alkylamino, dialkylamino, alkoxy, nitro, cyano, hydroxy, haloalkoxy and haloalkyl;
each R³⁰ and each R⁴⁰ is independently:
i) hydrogen;
ii) a phenyl group substituted with zero, one or two groups independently selected from halogen, alkyl, amino, alkylamino, dialkylamino, alkoxy, nitro, cyano, hydroxy, haloalkoxy or haloalkyl; or
iii) an alkyl group optionally substituted with halogen, hydroxyl, alkoxy, nitro, cyano, alkoxycarbonyl, alkylcarbonyl or haloalkoxy; and
each R³¹ is independently R³⁰, or —N(R³¹)₂ is an optionally substituted non-aromatic heterocyclic group; and
each R⁴¹ is independently R⁴⁰, or —N(R⁴¹)₂ is an optionally substituted non-aromatic heterocyclic group.

8. The compound of claim 7 wherein:
each R⁶ is independently halogen, nitro, cyano, C1-C2 alkyl, C1-C2 haloalkyl, C1-C2 alkoxy, C1-C2 haloalkoxy, —O—(CH₂)₂—N(R²¹)₂, —CH₂—N(R²¹)₂ or —CH₂CH₂—N(R²¹)₂; and
each R²¹ is independently —H or an alkyl group or —N(R²¹)₂ taken together is a five or six-membered non-aromatic nitrogen-containing heterocyclic group.

9. The compound of claim 8 wherein the compound is represented by the following formula: 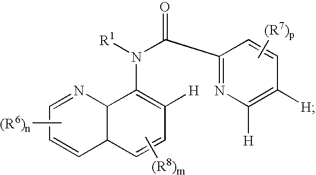
or a pharmaceutically acceptable salt thereof, wherein:
n, m and p are independently 0, 1, or 2;
each R³¹ is independently R³⁰; or —N(R³¹)₂ taken together is a monocyclic non-aromatic heterocyclic group, wherein:
i) the monocyclic non-aromatic heterocyclic group represented by —N(R³¹)₂ is optionally and independently substituted at any one or more substitutable ring carbon with C1-C2 alkyl, —OH, N-pyrrolidinyl, N-piperidinyl, N-morpholinyl, N-(4-methyl)-piperazinyl or N-pyrrolyl; and
ii) the monocyclic non-aromatic heterocyclic group represented by —N(R³¹)₂ is optionally and independently substituted at any substitutable ring nitrogen atom with C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy; and
each R⁴¹ is independently R⁴⁰; or —N(R⁴¹)₂ is a monocyclic non-aromatic heterocyclic group, wherein:
i) the monocyclic non-aromatic heterocyclic group represented by —N(R⁴¹)₂ is optionally and independently substituted at any one or more substitutable ring carbon with C1-C2 alkyl, —OH, N-pyrrolidinyl, N-piperidinyl, N-morpholinyl, N-(4-methyl)-piperazinyl or N-pyrrolyl; or
ii) the monocyclic non-aromatic heterocyclic group represented by —N(R⁴¹)₂ is optionally and independently substituted at any substitutable ring nitrogen atom with C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy.

10. The compound of claim 9 wherein:
each R³¹ is independently R³⁰; or —N(R³¹)₂ taken together is an optionally substituted N-piperidinyl, N-pyrrolidinyl, N-azepanyl, N-morpholinyl, N-thiomorphinyl, N′-substituted-N-piperazinyl or N′-substituted-N-diazepanyl group; and
each R⁴¹ is independently R⁴⁰; or —N(R⁴¹)₂ taken together is an optionally substituted N-piperidinyl, N-pyrrolidinyl, N-azepanyl, N-morpholinyl, N-thiomorphinyl, N-piperazinyl or N-diazepanyl.

11. The compound of claim 4 wherein the compound is represented by the following formula: 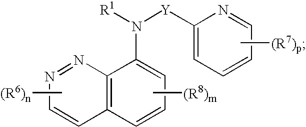
or a pharmaceutically acceptable salt thereof, wherein:
n is 0, 1 or 2;
m is 0, 1, 2 or 3;
each R⁸ is independently:
i) halogen, alkyl, haloalkyl, Ar², —OR⁴⁰, —O(haloalkyl), —SR⁴⁰, —NO₂, —CN, —N(R⁴¹)₂, —NR⁴¹C(O)R⁴⁰, —NR⁴¹CO₂R⁴², —N(R⁴¹)C(O)N(R⁴¹)₂, —C(O)R⁴⁰, —CO₂R⁴⁰, —C(O)N(R⁴¹)₂, —S(O)₂R⁴⁰, —SO₂N(R⁴¹)₂, —S(O)R⁴², —NR⁴¹SO₂N(R⁴¹)₂, —NR⁴¹SO₂R⁴², —V₂—Ar², —V₂—OR⁴⁰, —V₂—O(haloalkyl), —V₂—SR⁴⁰, —V₂—NO₂, —V₂—CN, —V₂—N(R⁴¹)₂, —V₂—NR⁴¹C(O)R⁴⁰, —V₂—NR⁴¹CO₂R⁴², —V₂—N(R⁴¹)C(O)N(R⁴¹)₂, —V₂—C(O)R⁴⁰, —V₂—CO₂R⁴⁰, —V₂—C(O)N(R⁴¹)₂, —V₂—S(O)₂R⁴⁰, —V₂—SO₂N(R⁴¹)₂, —V₂—S(O)R⁴², —V₂—NR⁴¹ SO₂N(R⁴¹)₂, —V₂—NR⁴¹SO₂R⁴², —O—V₂—Ar², —O—V₃—N(R⁴¹)₂, —S—V₂—Ar², —S—V₃—N(R⁴¹)₂, —N(R⁴¹)—V₂—Ar², —N(R⁴¹)—V₃—N(R⁴¹)₂, —NR⁴¹C(O)—V₂—N(R⁴¹)₂, —NR⁴¹C(O)—V₂—Ar², —C(O)—V₂—N(R⁴¹)₂, —C(O)—V₂—Ar², —CO₂—V₂—N(R⁴¹)₂, —CO₂—V₂—Ar², —C(O)N(R⁴¹)—V₃—N(R⁴¹)₂, —C(O)N(R⁴¹)—V₂—Ar , —S(O)₂—V₂—N(R⁴¹)₂, —S(O)₂—V₂—Ar², —SO₂N(R⁴¹)—V₃—-N(R⁴¹)₂, —SO₂N(R⁴¹)—V₂—Ar², —S(O)—V₂—N(R⁴¹)₂, —S(O)—V₂—Ar², —NR⁴¹SO₂—V₂—N(R⁴¹)₂ or —NR⁴¹SO₂—V₂—Ar²; or
ii) two adjacent R⁸ groups, taken together, form a methylenedioxy, ethylenedioxy or —[CH₂]₄— group; and
each V₂ is independently a C1-C4 alkylene group;
each V₃ is independently a C2-C4 alkylene group;
Ar² is a monocyclic aromatic group substituted with zero, one or two groups independently selected from halogen, alkyl, amino, alkylamino, dialkylamino, alkoxy, nitro, cyano, hydroxy, haloalkoxy or haloalkyl;
each R⁴⁰ is independently:
i) hydrogen;
ii) an aromatic substituted with zero, one or two groups independently selected from halogen, alkyl, amino, alkylamino, dialkylamino, alkoxy, nitro, cyano, hydroxy, haloalkoxy or haloalkyl; or
iii) an alkyl group optionally substituted with halogen, hydroxyl, alkoxy, nitro, cyano, alkoxycarbonyl, alkylcarbonyl or haloalkoxy; and
each R⁴¹ is independently R⁴⁰, —CO₂R⁴⁰, —SO₂R⁴⁰ or —C(O)R⁴⁰; or —N(R⁴¹)₂ taken togther is an optionally substituted non-aromatic heterocyclic group; and
each R⁴² is independently:
i) an aromatic substituted with zero, one or two groups independently selected from halogen, alkyl, amino, alkylamino, dialkylamino, alkoxy, nitro, cyano, hydroxy, haloalkoxy or haloalkyl; or
ii) an alkyl group optionally substituted with halogen, hydroxyl, alkoxy, nitro, cyano, alkoxycarbonyl, alkylcarbonyl or haloalkoxy.

12. The compound of claim 4 wherein the compound is represented by the following formula: 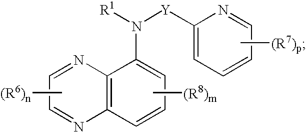
or a pharmaceutically acceptable salt thereof, wherein:
n is 0, 1 or 2;
m is 0, 1, 2 or 3;
each R⁸ is independently:
i) halogen, alkyl, haloalkyl, Ar², —OR⁴⁰, —O(haloalkyl), —SR⁴⁰, —NO₂, —CN, —N(R⁴¹)₂, —NR⁴¹C(O)R⁴⁰, —NR⁴¹CO₂R⁴², —N(R⁴¹)C(O)N(R⁴¹)₂, —C(O)R⁴⁰, —CO₂R⁴⁰, —C(O)N(R⁴¹)₂, —S(O)₂R⁴⁰, —SO₂N(R⁴¹)₂, —S(O)R⁴², —NR⁴¹SO₂N(R⁴¹)₂, —NR⁴¹SO₂R⁴², —V₂—Ar², —V₂—OR⁴⁰, —V₂—O(haloalkyl), —V₂—SR⁴⁰, —V₂—NO₂, —V₂—CN, —V₂—N(R⁴¹)₂, —V₂—NR⁴¹C(O)R⁴⁰, —V₂—NR⁴¹CO₂R⁴², —V₂—N(R⁴¹)C(O)N(R⁴¹)₂, —V₂—C(O)R⁴⁰, —V₂—CO₂R⁴⁰, —V₂—C(O)N(R⁴¹)₂, —V₂—S(O)₂R⁴⁰, —V₂—SO₂N(R⁴¹)₂, —V₂—S(O)R⁴², —V₂—NR⁴¹SO₂N(R⁴¹)₂, —V₂—NR⁴¹SO₂R⁴², —O—V₂—Ar², —O—V₃—N(R⁴¹)₂, —S—V₂—Ar₂, —S—V₃—N(R⁴¹)₂, —N(R⁴¹)—V₂—Ar², —N(R⁴¹)—V₃—N(R⁴¹)₂, —NR⁴¹C(O)—V₂—N(R⁴¹)₂, —NR⁴¹C(O)—V₂—Ar², —C(O)—V₂—N(R⁴¹)₂, —C(O)—V₂—Ar², —CO₂—V₂—N(R⁴¹)₂, —CO₂—V₂—Ar², —C(O)N(R⁴¹)—V₃—N(R⁴¹)₂, —C(O)N(R⁴¹)—V₂—Ar², —S(O)₂—V₂—N(R⁴¹)₂, —S(O)₂—V₂—Ar², —SO₂N(R⁴¹)—V₃—N(R⁴¹)₂, —SO₂N(R⁴¹)—V₂—Ar², —S(O)—V₂—N(R⁴¹)₂, —S(O)—V₂—Ar², —NR⁴¹SO₂—V₂—N(R⁴¹)₂ or —NR⁴¹SO₂—V₂—Ar²; or
ii) two adjacent R⁸ groups, taken together, form a methylenedioxy, ethylenedioxy or —[CH₂]₄— group; and
each V₂ is independently a C1-C4 alkylene group;
each V₃ is independently a C2-C4 alkylene group;
Ar² is a monocyclic aromatic group substituted with zero, one or two groups independently selected from halogen, alkyl, amino, alkylamino, dialkylamino, alkoxy, nitro, cyano, hydroxy, haloalkoxy or haloalkyl;
each R⁴⁰ is independently:
i) hydrogen;
ii) an aromatic substituted with zero, one or two groups independently selected from halogen, alkyl, amino, alkylamino, dialkylamino, alkoxy, nitro, cyano, hydroxy, haloalkoxy or haloalkyl; or
iii) an alkyl group optionally substituted with halogen, hydroxyl, alkoxy, nitro, cyano, alkoxycarbonyl, alkylcarbonyl or haloalkoxy; and
each R⁴¹ is independently R⁴⁰, —CO₂R⁴⁰, —SO₂R⁴⁰ or —C(O)R⁴⁰; or —N(R⁴¹)₂ taken togther is an optionally substituted non-aromatic heterocyclic group; and
each R⁴² is independently:
i) an aromatic substituted with zero, one or two groups independently selected from halogen, alkyl, amino, alkylamino, dialkylamino, alkoxy, nitro, cyano, hydroxy, haloalkoxy or haloalkyl; or
ii) an alkyl group optionally substituted with halogen, hydroxyl, alkoxy, nitro, cyano, alkoxycarbonyl, alkylcarbonyl or haloalkoxy.

13. The compound of claim 1 wherein R² and R³ are independently C1-C6 alkyl optionally substituted with amino, hydroxyl, methoxy or ethoxy.

14. The compound of claim 13 wherein Ar is an optionally substituted pyridyl group.

15. The compound of claim 14 wherein the compound is represented by the following formula: 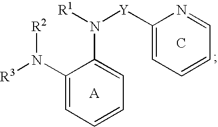
or a pharmaceutically acceptable salt thereof, wherein:
pyridyl Ring C is optionally substituted at any one or more substitutable ring carbon atoms and each Ring C substituent is independently selected; and
R¹is —H or C1-C3 alkyl.

16. The compound of claim 15 wherein the compound is represented by the following formula: 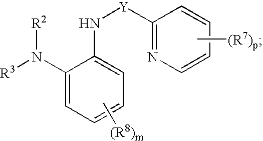
or a pharmaceutically acceptable salt thereof, wherein:
m and p are independently 0, 1, 2, 3 or 4;
each R⁷ is independently:
i) halogen, alkyl, haloalkyl, Ar¹, —OR³⁰, —O(haloalkyl), —SR³⁰, —NO₂, —CN, —N(R³¹)₂, —NR³¹C(O)R³⁰, —NR³¹CO₂R³², —N(R³¹)C(O)N(R³¹)₂, —C(O)R³⁰, —CO₂R³⁰, —S(O)₂R³⁰, —SO₂N(R³¹)₂, —S(O)R³², —NR³¹SO₂N(R³¹)₂, —NR³¹SO₂R³², —V—Ar¹, —V—OR³⁰, —V—O(haloalkyl), —V—SR³⁰, —V—NO₂, —V—CN, —V—N(R³¹)₂, —V—NR³¹C(O)R³⁰, —V—NR³¹CO₂R³², —V—N(R³¹)C(O)N(R³¹)₂, —V—C(O)R³⁰, —V—CO₂R³⁰, —V—S(O)₂R³⁰, —V—SO₂N(R³¹)₂, —V—S(O)R³², —V—NR³¹SO₂N(R³¹)₂, —V—NR³¹SO₂R³², —O—V—Ar¹, —O—V₁—N(R³¹)₂, —S—V—Ar¹, —S—V₁—N(R³¹)₂, —N(R³¹)—V—Ar¹, —N(R³¹)—V₁—N(R³¹)₂, —NR³¹C(O)—V—N(R³¹)₂, —NR³¹C(O)—V—Ar¹, —C(O)—V—N(R³¹)₂, —C(O)—V—Ar¹, —CO₂—V₁—N(R³¹)₂, —CO₂—V—Ar¹, —C(O)N(R³¹)—V₁—N(R³¹)₂, —C(O)N(R³¹)—V—Ar¹, —S(O)₂—V—N(R³¹)₂, —S(O)₂—V—Ar¹, —SO₂N(R³¹)—V₁—N(R³¹)₂, —SO₂N(R³¹)—V—Ar¹, —S(O)—V—N(R³¹)₂, —S(O)—V—Ar¹, —NR³¹SO₂—V—N(R³¹)₂ or —NR³SO₂—V—Ar¹; or
ii) two adjacent R⁷ groups, taken together, form a methylenedioxy, ethylenedioxy or —[CH₂]₄— group; and
each R⁸ is independently:
i) halogen, alkyl, haloalkyl, Ar², —OR⁴⁰, —O(haloalkyl), —SR⁴⁰, —NO₂, —CN, —N(R⁴¹)₂, —NR⁴¹C(O)R⁴⁰, —NR⁴¹CO₂R⁴², —N(R⁴¹)C(O)N(R⁴¹)₂, —C(O)R⁴⁰, —CO₂R⁴⁰, —C(O)N(R⁴¹)₂, —S(O)₂R⁴⁰, —SO₂N(R⁴¹)₂, —S(O)R⁴², —NR⁴¹SO₂N(R⁴¹)₂, —NR⁴¹SO₂R⁴², —V₂—Ar², —V₂—OR⁴⁰, —V₂—O(haloalkyl), —V₂—SR⁴⁰, —V₂—NO₂, —V₂—CN, —V₂—N(R⁴¹)₂, —V₂—NR⁴¹C(O)R⁴⁰, —V₂—NR⁴¹CO₂R⁴², —V₂—N(R⁴¹)C(O)N(R⁴¹)₂, —V₂—C(O)R⁴⁰, —V₂—CO₂R⁴⁰, —V₂—C(O)N(R⁴¹)₂, —V₂—S(O)₂R⁴⁰, —V₂—SO₂N(R⁴¹)₂, —V₂—S(O)R⁴², —V₂—NR⁴¹SO₂N(R⁴¹)₂, —V₂—NR⁴¹SO₂R⁴², —O—V₂—Ar², —O—V₃—N(R⁴¹)₂, —S—V₂—Ar², —S—V₃—N(R⁴¹)₂, —N(R⁴¹)—V₂—Ar², —N(R⁴¹)—V₃—N(R⁴¹)₂, —NR⁴¹C(O)—V₂—N(R⁴¹)₂, —NR⁴¹C(O)—V₂—Ar², —C(O)—V₂—N(R⁴¹)₂, —C(O)—V₂—Ar², —CO₂—V₃—N(R⁴¹)₂, —CO₂—V₂—Ar², —C(O)N(R⁴¹)—V₃—N(R⁴¹)₂, —C(O)N(R⁴¹)—V₂—Ar², —S(O)₂—V₂—N(R⁴¹)₂, —S(O)₂—V₂—Ar², —SO₂N(R⁴¹)—V₃—N(R⁴¹)₂, —SO₂N(R⁴¹)—V₂—Ar², —S(O)—V₂—N(R⁴¹)₂, —S(O)—V₂—Ar², —NR⁴¹SO₂—V₂—N(R⁴¹)₂ or —NR⁴¹SO₂—V₂—Ar²; or
ii) two adjacent R⁸ groups, taken together, form a methylenedioxy, ethylenedioxy or —[CH₂]₄— group;
each V and each V₂ is independently a C1-C4 alkylene group;
each V₁ and each V₃ is independently a C2-C4 alkylene group;
Ar¹ and Ar² are each independently a monocyclic aromatic group substituted with zero, one or two groups independently selected from halogen, alkyl, amino, alkylamino, dialkylamino, alkoxy, nitro, cyano, hydroxy, haloalkoxy or haloalkyl;
each R³⁰ and each R⁴⁰ is independently:
i) hydrogen;
ii) an aromatic group substituted with zero, one or two groups represented by halogen, alkyl, amino, alkylamino, dialkylamino, alkoxy, nitro, cyano, hydroxy, haloalkoxy or haloalkyl; or
iii) an alkyl group optionally substituted with halogen, hydroxyl, alkoxy, nitro, cyano, alkoxycarbonyl, alkylcarbonyl or haloalkoxy; and
each R³¹ is independently R³⁰, —CO₂R³⁰, —SO₂R³⁰ or —C(O)R³⁰; or —N(R³¹)₂ taken together is an optionally substituted non-aromatic heterocyclic group; and
each R⁴¹ is independently R⁴⁰, —CO₂R⁴⁰, —SO₂R⁴⁰ or —C(O)R⁴⁰; or —N(R⁴¹)₂ taken together s an optionally substituted non-aromatic heterocyclic group; and
each R³² and each R⁴² is independently:
i) an aromatic group substituted with zero, one or two groups represented by halogen, alkyl, amino, alkylamino, dialkylamino, alkoxy, nitro, cyano, hydroxy, haloalkoxy or haloalkyl; or
ii) an alkyl group optionally substituted with halogen, hydroxyl, alkoxy, nitro, cyano, alkoxycarbonyl, alkylcarbonyl or haloalkoxy.

17. The compound of claim 16 wherein:
each R⁷ is independently selected from halogen, C1-C4 alkyl, C1-C4 haloalkyl, cyano, —OR³⁰, —SR³⁰, —N(R³¹)₂, Ar¹, —V—OR³⁰, —V—N(R⁴¹)₂, —V—Ar¹, —O—V—Ar¹, —O—V₁—N(R³¹)₂, —S—V—Ar¹, —S—V₁—N(R³¹)₂, —N(R³¹)—V—Ar¹ or —N(R³¹)—V₁—N(R³¹)₂;
each R⁸ is independently selected from halogen, C1-C4 alkyl, C1-C4 haloalkyl, cyano, —OR⁴⁰, —SR⁴⁰, —N(R⁴¹)₂, Ar², —V₂—OR⁴⁰, —V₂—N(R⁴¹)₂, —V₂—Ar², —O—V₃—Ar², —O—V₃—N(R⁴¹)₂, —S—V₂—Ar², —S—V₃—N(R⁴¹)₂, —N(R⁴¹)—V₂—Ar² or —N(R⁴¹)—V₃—N(R⁴¹)₂;
Ar¹ and Ar² are phenyl, each independently substituted with zero, one or two groups independently selected from halogen, alkyl, amino, alkylamino, dialkylamino, alkoxy, nitro, cyano, hydroxy, haloalkoxy or haloalkyl;
each R³⁰ and each R⁴⁰ is independently:
i) hydrogen;
ii) a phenyl group substituted with zero, one or two groups independently selected from halogen, alkyl, amino, alkylamino, dialkylamino, alkoxy, nitro, cyano, hydroxy, haloalkoxy or haloalkyl; or
iii) an alkyl group optionally substituted with halogen, hydroxyl, alkoxy, nitro, cyano, alkoxycarbonyl, alkylcarbonyl or haloalkoxy; and
each R³¹ is independently R³⁰, or —N(R³¹)₂ taken together is an optionally substituted non-aromatic heterocyclic group;
each R⁴¹ is independently R⁴⁰, or —N(R⁴¹)₂ taken together is an optionally substituted non-aromatic heterocyclic group.

18. The compound of claim 17 wherein the compound is represented by the following formula: 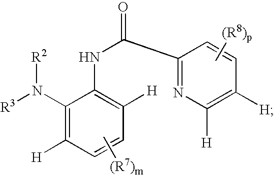
or a pharmaceutically acceptable salt thereof, wherein:
m and p are independently 0, 1, or 2;
each R³¹ is independently R³⁰; or —N(R³¹)₂ taken together is a monocyclic non-aromatic heterocyclic group, wherein:
i) the monocyclic non-aromatic heterocyclic group represented by —N(R³¹)₂ is optionally and independently substituted at any one or more substitutable ring carbon with C1-C2 alkyl, —OH, N-pyrrolidine, N-piperidinyl, N-morpholinyl, N-(4-methyl)-piperazinyl or N-pyrrolyl; and
ii) the monocyclic non-aromatic heterocyclic group represented by —N(R³¹)₂ is optionally and independently substituted at any substitutable ring nitrogen atom with C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy; and
each R⁴¹ is independently R⁴⁰; or —N(R⁴¹)₂ taken together is a monocyclic non-aromatic heterocyclic group, wherein:
a) the monocyclic non-aromatic heterocyclic group represented by —N(R⁴¹)₂ is optionally and independently substituted at any one or more substitutable ring carbon with C1-C2 alkyl, —OH, N-pyrrolidine, N-piperidinyl, N-morpholinyl, N-(4-methyl)-piperazinyl or N-pyrrolyl; and
b) the monocyclic non-aromatic heterocyclic group represented by —N(R⁴¹)₂ is optionally and independently substituted at any substitutable ring nitrogen atom with C1-C2 alkyl, C1-C2 hydroxyalkyl, or benzyl optionally substituted with halogen, nitro, cyano, C1-C2 alkyl, C1-C2 haloalkyl, C1-C2 alkoxy or C1-C2 haloalkoxy.

19. The compound of claim 18 wherein:
each R³¹ is independently R³⁰; or —N(R³¹)₂ taken together is an optionally substituted N-piperidinyl, N-pyrrolidinyl, N-azepanyl, N-morpholinyl, N-thiomorphinyl, N-piperazinyl or N-diazepanyl group; and
each R⁴¹ is independently R⁴⁰; or —N(R⁴¹)₂ taken together is an optionally substituted N-piperidinyl, N-pyrrolidinyl, N-azepanyl, N-morpholinyl, N-thiomorphinyl, N-piperazinyl or N-diazepanyl.

20. The compound of claim 1 wherein the compound is Compound 2, Compound 9, Compound 13, Compound 19, Compound 20, Compound 30, Compound 34, Compound 38, Compound 40, Compound 41, Compound 42, Compound 46, Compound 50, Compound 53, Compound 54, Compound 98, Compound 99, Compound 100, Compound 101, Compound 102 and Compound 103 or a pharmaceutically acceptable salt thereof.

21. A compound of represented by the following formula: 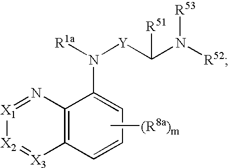
or a pharmaceutically acceptable salt thereof, wherein:
m is 0, 1, 2 or 3;
—Y— is —SO₂— or —C(O)—;
R^{1a} is H or a C1-C6 alkyl group optionally substituted with amino, hydroxyl, methoxy or ethoxy;
X₁—X₃ are independently N, CH, or CR^{6a}, provided that X₁ and X₂ are not both N;
each R^{6a} is independently halogen, alkyl, haloalkyl, —OR²⁰, —O(haloalkyl), —SR²⁰, —NO₂, —CN, —N(R²¹)₂, —NR²¹C(O)R²⁰, —NR²¹CO₂R²², —N(R²¹)C(O)N(R²¹)₂, —C(O)R²⁰, —C(O)N(R²¹)₂, —S(O)₂R²⁰, —SO₂N(R²¹)₂, —S(O)R²², —NR²¹SO₂N(R²¹)₂, —NR²¹SO₂R²², —Vₒ—N(R²¹)₂, —O—Vₒₒ—N(R²¹)₂, —S—Vₒₒ—N(R²¹)₂ or —N(R²¹)—Vₒₒ—N(R²¹)₂;
each Vₒ is independently a C1-C4 alkylene group;
each Vₒₒ is independently a C2-C4 alkylene group;
each R^{8a} is independently:
i) halogen, alkyl, haloalkyl, Ar², —O(haloalkyl), —SR⁴⁰, —NO₂, —CN, —N(R⁴¹)₂, —NR⁴¹C(O)R⁴⁰, —NR⁴¹CO₂R⁴², —N(R⁴¹)C(O)N(R⁴¹)₂, —C(O)R⁴⁰, —C(O)N(R⁴¹)₂, —S(O)₂R⁴⁰, —SO₂N(R⁴¹)₂, —S(O)R⁴², —NR⁴¹SO₂N(R⁴¹)₂, —NR⁴¹SO₂R⁴², —V₂—Ar², —V₂—O(haloalkyl), —V₂—OR⁴⁰, —V₂—SR⁴⁰, —V₂—NO₂, —V₂—CN, —V₂—N(R⁴¹)₂, —V₂—NR⁴¹C(O)R⁴⁰, —V₂—NR⁴¹CO₂R⁴², —V₂—N(R⁴¹)C(O)N(R⁴¹)₂, —V₂—C(O)R⁴⁰, —V₂—CO₂R⁴⁰, —V₂—C(O)N(R⁴¹)₂, —V₂—S(O)₂R⁴⁰, —V₂—SO₂N(R⁴¹)₂, —V₂—S(O)R⁴², —V₂—NR⁴¹SO₂N(R⁴¹)₂, —V₂—NR⁴¹SO₂R⁴², —O—V₂—Ar², —O—V₃—N(R⁴¹)₂, —S—V₂—Ar², —S—V₃—N(R⁴¹)₂, —N(R⁴¹)—V₂—Ar², —N(R⁴¹)—V₃—N(R⁴¹)₂, —NR⁴¹C(O)—V₂—N(R⁴¹)₂, —NR⁴¹C(O)—V₂—Ar², —C(O)—V₃—N(R⁴¹)₂, —C(O)—V₂—Ar², —CO₂—V₃—N(R⁴¹)₂, —CO₂—V₂—Ar², —C(O)N(R⁴¹)—V₃—N(R⁴¹)₂, —C(O)N(R⁴¹)—V₂—Ar², —S(O)₂—V₂—N(R⁴¹)₂, —S(O)₂—V₂—Ar², —SO₂N(R⁴¹)—V₃—N(R⁴¹)₂, —SO₂N(R⁴¹)—V₂—Ar², —S(O)—V₂—N(R⁴¹)₂, —S(O)—V₂—Ar , —NR⁴¹SO₂—V₂—N(R⁴¹)₂ or —NR⁴¹SO₂—V₂—Ar²; or
ii) two adjacent R⁸ groups, taken together, form a methylenedioxy, ethylenedioxy or —[CH₂]₄— group; and
each V₂ is independently a C1-C4 alkylene group;
each V₃ is independently a C2-C4 alkylene group;
each R²⁰ is independently hydrogen or an alkyl group; and
each R²¹ is independently hydrogen, an alkyl group, —CO₂R²⁰, —SO₂R²⁰ or —C(O)R²⁰; or —N(R²¹)₂ taken together is an optionally substituted non-aromatic nitrogen-containing heterocyclic group; and
each R²² is independently an alkyl group;
Ar² is a monocyclic aromatic group substituted with zero, one or two groups independently selected from halogen, alkyl, amino, alkylamino, dialkylamino, alkoxy, nitro, cyano, hydroxy, haloalkoxy or haloalkyl;
each R⁴⁰ is independently:
i) hydrogen;
ii) an aromatic substituted with zero, one or two groups independently selected from halogen, alkyl, amino, alkylamino, dialkylamino, alkoxy, nitro, cyano, hydroxy, haloalkoxy or haloalkyl; or
iii) an alkyl group optionally substituted with halogen, hydroxyl, alkoxy, nitro, cyano, alkoxycarbonyl, alkylcarbonyl or haloalkoxy; and
each R⁴¹ is independently R⁴⁰, —CO₂R⁴⁰, —SO₂R⁴⁰ or —C(O)R⁴⁰; or —N(R⁴¹)₂ taken together is an optionally substituted non-aromatic heterocyclic group; and
each R⁴² is independently:
i) an aromatic substituted with zero, one or two groups independently selected from halogen, alkyl, amino, alkylamino, dialkylamino, alkoxy, nitro, cyano, hydroxy, haloalkoxy or haloalkyl; or
ii) an alkyl group optionally substituted with halogen, hydroxyl, alkoxy, nitro, cyano, alkoxycarbonyl, alkylcarbonyl or haloalkoxy; and
wherein:
i) R⁵¹ and R⁵², taken together with their intervening atoms, form an optionally substituted monocyclic non-aromatic heterocyclic group and R⁵³ is —H or C1-C6 alkyl group optionally substituted with amine, C1-C2 alkylamine, C1-C2 dialkylamine, hydroxyl, methoxy, ethoxy, cycloalkyl, or an optionally substituted aryl group; or
ii) R⁵¹ is C1-C6 alkyl group optionally substituted with amino, hydroxyl, methoxy, ethoxy or an optionally substituted aryl group and —NR⁵²R⁵³ is an optionally substituted mono-cyclic non-aromatic heterocyclic group; or
iii) R⁵s is an optionally substituted C1-C3 aralkyl and R⁵² and R⁵³ are independently —H or an optionally substituted C1-C6 alkyl group.

22.-40. (canceled)

41. A method of inhibiting angiogenesis in a subject in need of such treatment, said method comprising the step of administering to the subject an effective amount of a compound represented by the following formula: 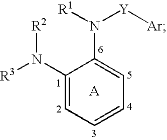
or a pharmaceutically acceptable salt thereof, wherein:
Ring A is optionally substituted at any one or more substitutable ring carbon atoms;
—Y— is —SO₂— or —C(O)—;
Ar is an optionally substituted monocyclic aryl group;
R¹is H or a C1-C6 alkyl group optionally substituted with amino, hydroxyl, methoxy or ethoxy;
R² and R³ are independently C1-C6 alkyl optionally substituted with amino, hydroxyl, methoxy or ethoxy; or
R² is absent and R³, taken together with the nitrogen atom to which it is bonded and C1 and C2 of Ring A, forms a optionally substituted six-membered- nitrogen-containing aromatic group referred to as “Ring B”; and
wherein each substituent on Ring A, B and Ar is independently selected.

42.-65. (canceled)

66. A method of inhibiting angiogenesis in a subject in need of such treatment, said method comprising the step of administering to the subject an effective amount of a compound represented by the following formula: 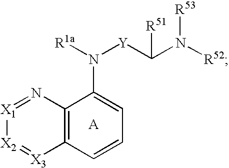
or a pharmaceutically acceptable salt thereof, wherein:
Ring A is optionally substituted an any substitutable ring carbon atom;
Y is —C(O)— or —S(O)₂—;
X₁—X₃ are independently N, CH or CR^{6a}, provided that X₁ and X₂ are not both N; and
R^{1a} is H or a C1-C6 alkyl group optionally substituted with amino, hydroxyl, methoxy or ethoxy;
each R^{6a} is independently halogen, alkyl, haloalkyl, —OR²⁰, —O(haloalkyl), —SR²⁰, —NO₂, —CN, —N(R²¹)₂, —NR²¹C(O)R²⁰, —NR²¹CO₂R²², —N(R²¹)C(O)N(R²¹)₂, —C(O)R²⁰, —CO₂R²⁰, —C(O)N(R²¹)₂, —S(O)₂R²⁰, —SO₂N(R²¹)₂, —S(O)R²², —NR²¹SO₂N(R²¹)₂, —NR²¹SO₂R²², —Vₒ—N(R²¹)₂, —O—Vₒₒ—N(R²¹)₂, —S—Vₒₒ—N(R²¹)₂or —N(R²¹)—Vₒₒ—N(R²¹)₂;
each Vₒ is independently a C1-C4 alkylene group;
each Vₒₒ is independently a C2-C4 alkylene group;
each R²⁰ is independently hydrogen or an alkyl group; and
each R²¹ is independently hydrogen, an alkyl group, —CO₂R²⁰, —SO₂R²⁰ or —C(O)R²⁰; or —N(R²¹)₂ taken together is an optionally substituted non-aromatic nitrogen-containing heterocyclic group; and
each R²² is independently an alkyl group; and wherein:
i) R⁵¹ and R⁵² taken together with their intervening atoms form an optionally substituted monocyclic non-aromatic heterocyclic group and R⁵³ is —H or C1-C6 alkyl group optionally substituted with amine, C1-C2 alkylamine, C1-C2 dialkylamine, hydroxyl, methoxy, ethoxy, cycloalkyl, or optionally substituted aryl; or
ii) R⁵¹ is —H or C1-C6 alkyl group optionally substituted with amino, hydroxyl, methoxy, ethoxy or an optionally substituted aryl group; and R⁵² and R⁵³ are independently —H or C1-C6 alkyl group optionally substituted with amino, hydroxyl, methoxy, ethoxy, cycloalkyl, or an optionally substituted aryl group; or —NR⁵²R⁵³ is an optionally substituted monocyclic non-aromatic heterocyclic group.

67.-91. (canceled)
